(19) United States
(12) Patent Application Publication
(10) Pub. No.: US 2005/0176767 A1
(43) Pub. Date: Aug. 11, 2005

(54) Pyridine carboxamide and methods for inhibiting HIV integrase

(76) Inventors: Laval Chan Chun Kong, Kirkland (CA); Ming-Qiang Zhang, Cherry Hinton (GB); Liliane Halab, Laval (CA); Nghe Nguyen-Ba, Laprairie (CA); Bingcan Liu, St. Laurent (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/976,238

(22) Filed: Oct. 29, 2004

Related U.S. Application Data
(60) Provisional application No. 60/515,443, filed on Oct. 30, 2003.

(51) Int. Cl. A61K 31/4545; A61K 31/444; A61K 31/4439; C07D 43/02; C07D 417/02

(52) U.S. Cl. 514/318; 514/340; 514/355; 546/193; 546/315; 546/269.4

(57) ABSTRACT

Compounds of formula I: 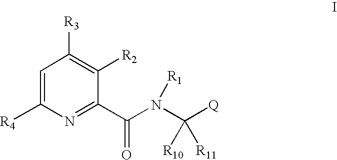
wherein R₁, R₂, R₄, R₁₀, R₁₁, and Q are as defined herein, and their pharmaceutically acceptable salts, are useful in the prevention or treatment of HIV infections.

This application claims the benefit of U.S. provisional application Ser. No. 60/515,443, filed Oct. 30, 2003, the entire disclosure of which is hereby incorporated by reference.

### FIELD OF THE INVENTION

The present invention relates to novel compounds and method for the treatment or prevention of HIV infection/AIDS.

### BACKGROUND OF THE INVENTION

HIV integrase is an attractive therapeutic target for the development of drugs to treat HIV infection (Pommier Y et al: Antiviral Chem Chemother 1997, 8, 463-83; De Clercq, E: Med Res Rev 2002, 22, 531-565; Nair V: Rev. Med. Virol. 2002, 12, 179-193). It is a protein of Mr 32000 encoded at the 3′-end of pol gene. This viral enzyme catalyses the integration of viral DNA into host cell chromosomal DNA to form a provirus. This essential step in the viral life cycle proceeds by integrase recognizing and binding to attachment sites located at the ends of linear viral DNA, followed by the cleavage of highly conserved CA dinucleotides from the 5′ and 3′ long-terminal repeats. This reaction, known as 3′-processing, occurs in the cytoplasm and exposes the 3′-OH group from the CA unit. This OH group subsequently acts as a nucleophile by attacking the host DNA in a transesterification reaction. This second reaction, referred to as strand transfer or integration, occurs in the nucleus. These reactions are adequately represented in vitro using purified integrase, a double-stranded DNA template matching the viral DNA ends as a substrate surrogate along with a divalent metal ion (Mn²⁺ or Mg²⁺) cofactor. It has been reported that selective inhibition of strand transfer reaction results in the inhibition of HIV viral replication (Pais G C G & Burke T R Jr: Drugs of the Future, 2002, 27, 1101-1111).

HIV integrase is further attractive as a target for the development of anti-HIV drugs because there is apparently no functional equivalent of this enzyme in human cells. It has also bee reported that integrase inhibitors in combination with either reverse transcriptase or protease inhibitors are potently synergistic against both wild-type HIV and reverse transcriptase inhibitor resistant viruses (Robinson W E Jr et al Antiviral Res. 1998, 39, 101-11; Beale K et al Antiviral Res 2000, 46, 223-232).

A number of integrase inhibitors have been reported, including nucleotide-based inhibitors, DNA binders, catechols, hydrazides, etc (Neamati N: Expert Opin Ther Patents 2002, 12, 709-724). Most of these compounds inhibit integrase function in extracellular oligonucleotide assays but often lack inhibitory potency when assayed using fully assembled preintegration complexes or fail to show antiviral effects against HIV-infected cells. A class of diketo-containing integrase inhibitors has been found to inhibit viral replication by blocking the strand transfer step of integrase reactions (Pais G C G & Burke T R Jr: Drugs of the Future, 2002, 27, 1101-1111). An inhibitor of this class has been in clinical trials for the treatment of HIV infection (Billich A: Curr Opin Investig Drugs 2003, 4, 206-209). However, in spite of their high integrase inhibitory potencies, diketo-containing compounds are electrophilic and they bind covalently to human cellular proteins leading to potential cytotoxicity. In addition, it has been reported recently that some diketo-containing compounds interfere with DNA cleavage and disintegration activities of RAG1/2 which are essential for the development of mammalian immune system (Melek M et al: Proc Natl Acad Sci USA 2002, 99, 134-7).

### SUMMARY OF THE INVENTION

In accordance with the present invention, there is provided a compound of formula I: 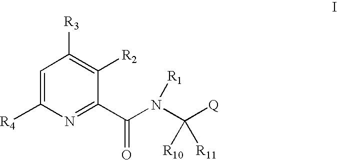
or a pharmaceutically acceptable salt thereof wherein,
R₁ is hydrogen or C₁₋₁₀ alkyl;
R₂ is hydroxyl, C₁₋₁₀ alkoxy or C₆aryl-C₁₋₁₀ alkyloxy;
R₃ is amino, amido, sulfonamido, azido, hydroxyl, halogen, cyano, carboxy, C₁₋₁₀ alkoxy, 5-6 membered heterocycle, C₆₋₁₀ aryl-C₁₋₁₀ alkyloxy, C₁₋₁₀ alkyl, or SOₙR₁₂ (n=0, 1, 2);
R₄ is hydrogen, halogen, hydroxyl, carboxy, C₁₋₁₀ alkyl, amino, amido, sulfonamide, SOₙR₁₂ (n=0, 1, 2), C₁₋₁₀ alkoxy, C₆₋₁₀ aryl, 5-6 membered heterocycle, or C₅₋₁₀ heteroaryl;
R₁₀, R₁₁, R₁₂ are each independently hydrogen, C₁₋₁₀ alkyl, C₆₋₁₀ aryl, or C₇₋₁₂aralkyl;
Q is optionally substituted phenyl, C₁₋₁₀ alkyl, 5-6 membered heterocycle, or C₁₋₁₂aralkyl;
   with the proviso that when R₃ is methoxy, R₂ is hydroxyl, R₁ is hydrogen and R₄ is hydrogen then Q is phenyl substituted by at least 3 substituents.

In one embodiment, there is provided a method of preventing or treating HIV infection in a subject which comprises administering to the subject a therapeutically effective amount of a compound, a combination or a pharmaceutical composition of the present invention.

In one embodiment, there is provided a method of preventing, delaying or treating AIDS in a subject which comprises administering to the subject a therapeutically effective amount of a compound, a combination or a pharmaceutical composition of the present invention.

In one embodiment, there is provided a method of inhibiting HIV integrase in a subject which comprises administering to the subject a therapeutically effective amount of a compound a combination or a pharmaceutical composition of the present invention.

In one embodiment, there is provided a method of preventing integration of HIV DNA into host cell DNA in a subject which comprises administering to the subject a therapeutically effective amount of a compound, a combination or a pharmaceutical composition of the present invention.

In one embodiment, there is provided a method of preventing the HIV DNA strand transfer to the host cell DNA in a subject which comprises administering to the subject a therapeutically effective amount of a compound, a combination or a pharmaceutical composition of the present invention.

In another embodiment, the invention provides the use of a compound or combination of the present invention for the manufacture of a medicament for preventing or treating HIV infection or preventing, delaying or treating AIDS.

In another embodiment, the invention provides the use of a compound or combination of the present invention for the manufacture of a medicament for preventing anyone of HIV replication, integration of HIV DNA into host cell DNA, 3′-end processing of HIV DNA or HIV DNA strand transfer to the host cell DNA.

In another aspect, the present invention provides a combination comprising a therapeutically effective amount of compound of the present invention, and a therapeutically effective amount of at least one antiviral agent.

A further aspect of the invention is therefore presented as a pharmaceutical composition comprising a compound or combination of the present invention together with at least one pharmaceutically acceptable carrier or excipient thereof.

### DETAILED DESCRIPTION OF THE INVENTION

In one embodiment, compounds of the present invention comprise those wherein the following embodiments are present, either independently or in combination.

In one embodiment, there is provided a compound of formula I: 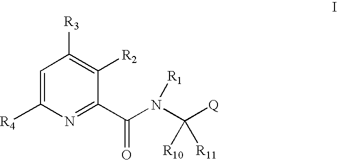
or pharmaceutically acceptable salt thereof wherein, R₁, R₂, R₃, R₄, R₁₀, R₁₁, and Q are as defined above.

In another embodiment, there is provided a compound of formula I, or a pharmaceutically acceptable salt, wherein:
R₁ is hydrogen or C₁₋₆ alkyl;
R₂ is hydroxyl, C₁₋₆ alkoxy or C₆aryl-C₁₋₆ alkyloxy;
R₃ is amino, azido, hydroxyl, halogen, cyano, carboxy, C₁₋₆ alkoxy, 5-6 membered heterocycle, or C₆aryl-C₁₋₆ alkyloxy;
R₄ is hydrogen, halogen, hydroxyl, carboxy, C₁₋₆ alkyl, C₁₋₆ alkoxy, 5-6 membered heterocycle, or C₆₋₁₀ aryl;
R₁₀, R₁₁, R₁₂ are each independently hydrogen or C₁₋₁₀ alkyl; and
Q is optionally substituted phenyl;
   with the proviso that when R₃ is methoxy, R₂ is hydroxyl, R₁ is hydrogen and R₄ is hydrogen then Q is phenyl substituted by at least 3 substituents.

In one embodiment, R₁ is hydrogen or C₁₀ alkyl.

In one embodiment, R₁ is hydrogen.

In one embodiment, R₁ is C₁₋₁₀ alkyl.

In one embodiment, R₁ is C₁₋₃ alkyl.

In a further embodiment, R₁ is a C₁₋₁₀ alkyl selected from methyl, ethyl, propyl, isopropyl, cyclopropyl and cyclohexyl.

In one embodiment, R₂ is hydroxyl, C₁₋₁₀ alkoxy or C₆aryl-C₁₋₁₀ alkyloxy.

In one embodiment, R₂ is hydroxyl or C₁₋₁₀ alkoxy.

In further embodiments:
R₂ is hydroxyl;
R₂ is C₁₋₁₀ alkoxy;
R₂ is C₁₋₃ alkoxy;
R₂ is a C₁₋₁₀ alkoxy selected from methoxy, ethyloxy, propyloxy, isopropyloxy, cyclopropyloxy and cyclohexyloxy;
R₂ is methoxy;
R₂ is C₆aryl-C₁₋₁₀ alkyloxy;
R₂ is benzyloxy.

In one embodiment, R₃ is amino, amido, sulfonamido, azido, hydroxyl, halogen, cyano, carboxyl, C₁₋₁₀ alkoxy, 5-6 membered heterocycle, C₆aryl-C₁₋₁₀ alkyloxy, C₁₋₁₀ alkyl, or SOₙR₁₂ (n=0, 1, 2);

In one embodiment, R₃ is hydroxyl, halogen, C₁₋₁₀ alkoxy or 5-6 membered heterocycle.

In further embodiments:

R₃ is C₁₋₃ alkoxy;
R₃ is a C₁₋₆ alkoxy selected from methoxy, ethyloxy, propyloxy, isopropyloxy, cyclopropyloxy and cyclohexyloxy;
R₃ is methoxy;
R₃ is amino;
R₃ is azido;
R₃ is hydroxyl;
R₃ is halogen;
R₃ is cyano;
R₃ is carboxy;
R₃ is amido;
R₃ is alkyl;
R₃ is sulfonamido;
R₃ is SOₙR₁₂ (n=0, 1, 2,);
R₃ is 5-6 membered heterocycle;
R₃ is pyridinyl, thiazolyl, furanyl, thienyl or piperidinyl;
R₃ is 2-pyridinyl, 2-thiazolyl, 2-furanyl, 2-thienyl or 1-piperidinyl;
R₃ is benzyloxy.

In one embodiment, R₄ is hydrogen, halogen, hydroxyl, carboxy, C₁₋₁₀ alkyl, amino, amido, sulfonamide, SOₙR₁₂ (n=0, 1, 2), C₁₋₁₀ alkoxy, 5-6 membered heterocycle, or C₅₋₁₀ heteroaryl;

In further embodiments:
R₄ is halogen, C₁₋₁₀ alkyl, C₁₋₁₀ alkoxy or 5-6 membered heterocycle;
R₄ is halogen;
R₄ is bromide;
R₄ is C₁₋₁₀ alkyl;
R₄ is C₁₋₃ alkyl;
R₄ is a C₁₋₁₀ alkyl selected from methyl, ethyl, propyl, isopropyl, vinyl, 1,2-dihydroxyethyl, hydroxymethyl, methyloxymethyl, cyclopropyl and cyclohexyl;
R₄ is a C₁₋₁₀ alkyl selected from methyl, ethyl, vinyl, 1,2-dihydroxyethyl, hydroxymethyl and methyloxymethyl;
R₄ is hydroxyl;
R₄ is carboxy,
R₄ is aryl;
R₄ is amino;
R₄ is amido;
R₄ is sulfonamido;
R₄ is SOₙR₁₂;
R₄ is C₁₋₁₀ alkoxy;
R₄ is C₁₋₃ alkoxy;
R₄ is a C₁₋₁₀ alkoxy selected from methoxy, ethyloxy, propyloxy, isopropyloxy, cyclopropyloxy and cyclohexyloxy;
R₄ is methoxy;
R₄ is 5-6 membered heterocycle;
R₄ is 5-6 membered heterocycle selected from furanyl, tetrahydrofuranyl, thienyl, thiazolyl, pyridinyl, 2,2-dimethyl[1,3]dioxolanyl and piperidinyl;
R₄ is tetrahydrofuranyl.

In one embodiment, R₁₀ and R₁₁, are each independently selected from hydrogen or C₁₋₁₀ alkyl.

In further embodiments:

R₁₀ and R₁₁ are each hydrogen;
R₁₀ and R₁₁ are each C₁₁, alkyl;
R₁₀ is hydrogen and R₁₁ is C₁₋₁₀ alkyl;
R₁₀ is hydrogen and R₁₁ is methyl;
R₁₀ and R₁₁ are each methyl;
R₁₀ and R₁₁ are C₁₋₃ alkyl.

In one embodiment, Q is optionally substituted phenyl, C₁₋₁₀ alkyl, 5-6 membered heterocycle or C₇₋₁₂aralkyl.

In one embodiment, Q is phenyl optionally substituted by one or more substituent.

In one embodiment, Q is C₁₋₁₀ alkyl.

In one embodiment, Q is cyclohexyl.

In one embodiment, Q is 5-6 membered heterocycle.

In one embodiment, Q is 2-pyridinyl.

In one embodiment, Q is C₇₋₁₂aralkyl.

In one embodiment, Q is benzyl.

In one embodiment, Q is phenyl.

In one embodiment, Q is phenyl substituted by one or more substituents independently selected from halogen, amino, amidino, amido, azido, cyano, guanidino, hydroxyl, nitro, nitroso, urea, OS(O)₂Rₘ (wherein Rₘ is C₁₋₁₀ alkyl, C₆₋₁₀ aryl or 3-10 membered heterocycle), OS(O)₂OR, (wherein R, is H, C₁₋₁₀ alkyl, C₆₋₁₀ aryl or 3-10 membered heterocycle), S(O)₂ORₚ (wherein Rₚ is H, C₁₋₁₀ alkyl, C₆₋₁₀ aryl or 3-10 membered heterocycle), S(O)₀₋₂R_{q} (wherein R_{q} is H, C₁₋₁₀ alkyl, C₆₋₁₀ aryl or 3-10 membered heterocycle), OP(O)ORₛORₜ, P(O)ORₛORₜ (wherein Rₛ and Rₜ are each independently H or C₁₋₁₀ alkyl), C₁₋₁₀ alkyl, C₆₋₁₂aralkyl (e.g. C₁₋₁₂aralkyl), C₆₋₁₀aryl, C₁₋₁₀alkoxy, C₆₋₁₂ aralkyloxy (e.g. C₁₋₁₂aralkyloxy), C₆₋₁₀ aryloxy, 3-10 membered heterocycle, C(O)Rᵤ (wherein Rᵤ is H, C₁₋₁₀ alkyl, C₆₋₁₀ aryl, C₆₋₁₂ aralkyl (e.g. C₇₋₁₂aralkyl) or 3-10 membered heterocycle), C(O)ORᵥ (wherein Rᵥ is H, C₁₋₁₀ alkyl, C₆₋₁₀ aryl, C₆₋₁₂ aralkyl (e.g. C₇₋₁₂aralkyl) or 3-10 membered heterocycle), NRₓC(O)R_{w} (wherein Rₓ is H or C₁₋₁₀ alkyl and R_{w} is H, C₁₋₁₀ alkyl, C₆₋₁₀ aryl, C₆₋₁₂ aralkyl (e.g. C₇₋₁₂aralkyl) or 3-10 membered heterocycle, or Rₓ and R_{w} are taken together with the atoms to which they are attached to form a 3 to 10 membered heterocycle) and SO₂NR_{y}R_{z} (wherein R_{y} and R_{z} are each independently H, C₁₋₁₀ alkyl, C₆₋₁₀ aryl, C₃₋₁₀ heterocycle or C₆₋₁₂ aralkyl (e.g. C₇₋₁₂aralkyl)).

In one embodiment, Q is phenyl substituted by one or more substituents independently selected from halogen, amino, amido, azido, cyano, hydroxyl, urea, S(O)₂ORₚ (wherein Rₚ is H, C₁₋₁₀ alkyl, C₆₋₁₀ aryl or 3-10 membered heterocycle), S(O)₂R_{q} (wherein R_{q} is H, C₁₋₁₀ alkyl, C₆₋₁₀ aryl or 3-10 membered heterocycle), P(O)ORₛORₜ (wherein Rₛ and Rₜ are each independently H or C₁₋₁₀ alkyl), C₁₋₁₀ alkyl, C₁₋₁₀ alkoxy, C(O)Rᵤ (wherein Rᵤ is H, C₁₋₁₀ alkyl, C₆₋₁₀ aryl, C₆₋₁₂ aralkyl (e.g. C₇₋₁₂ aralkyl) or 3-10 membered heterocycle), C(O)ORᵥ (wherein Rᵥ is H, C₁₋₁₀ alkyl, C₆₋₁₀ aryl, C₆₋₁₂ aralkyl (e.g. C₇₋₁₂aralkyl) or 3-10 membered heterocycle), NRₓC(O)R_{w} (wherein Rₓ is H or C₁₋₁₀alkyl and R_{w} is H, C₁₋₁₀ alkyl, C₆₋₁₀ aryl, C₆₋₁₂ aralkyl (e.g. C₇₋₁₂aralkyl) or 3-10 membered heterocycle, or Rₓ and R_{w} are taken together with the atoms to which they are attached to form a 3 to 10 membered heterocycle) and SO₂NR_{y}R_{z} (wherein R_{y} and R_{z} are each independently H, C₁₋₁₀ alkyl, C₆₋₁₀ aryl, C₃₋₁₀ heterocycle or C₆₋₁₂ aralkyl (e.g. C₇₋₁₂aralkyl)).

In one embodiment, Q is phenyl substituted by one or more substituents independently selected from halogen, amino, amido, azido, cyano, hydroxyl, urea, S(O)₂ORₚ (wherein Rₚ is H or C₁₋₁₀ alkyl), S(O)₂R_{q} (wherein R_{q} is H or C₁₋₁₀ alkyl), P(O)ORₛORₜ (wherein Rₛ and Rₜ are each independently H or C₁₋₁₀ alkyl), C₁₋₁₀alkyl, C₁₋₁₀alkoxy, C(O)Rᵤ (wherein Rᵤ is H or C₁₋₁₀ alkyl), C(O)ORᵥ (wherein Rᵥ is H, or C₁₋₁₀ alkyl), NRₓC(O)R_{w} (wherein Rₓ is H or C₁₋₁₀ alkyl and R_{w} is H or C₁₋₁₀ alkyl) and SO₂NR_{y}R_{z} (wherein R_{y} and R_{z} are each independently H or C₁₋₁₀ alkyl).

In one embodiment, Q is phenyl substituted by one or more substituents independently selected from halogen, amino, amido, azido, cyano, hydroxyl, C₁₋₁₀alkyl, C₁₋₁₀alkoxy, C(O)Rᵤ (wherein Rᵤ is selected from H or C₁₋₁₀ alkyl), C(O)ORᵥ (wherein Rᵥ is selected from H or C₁₋₁₀ alkyl), or SO₂NR_{y}R_{z} (wherein R_{y} and R_{z} are each independently selected from H or C₁₋₁₀ alkyl).

In one embodiment, Q is phenyl substituted by one or more substituents independently selected from halogen, amino, amido, cyano, hydroxyl, C₁₋₁₀alkyl, C₁₋₁₀alkoxy, C(O)Rᵤ (wherein Rᵤ is H or C₁₋₁₀ alkyl), C(O)ORᵥ (wherein Rᵥ is H or C₁₋₁₀ alkyl), and SO₂NR_{y}R_{z} (wherein R_{y} and R_{z} are each independently H or C₁₋₁₀ alkyl).

In one embodiment, Q is 4-fluorophenyl.

In a further embodiment, there is provided compound of formula I: 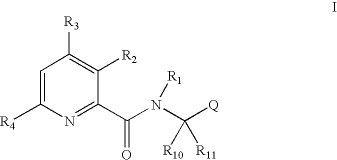
or a pharmaceutically acceptable salt thereof wherein,
R₁ is hydrogen or C₁₋₁₀ alkyl;
R₂ is hydroxyl, C₁₋₁₀ alkoxy or C₆aryl-C₁₋₁₀ alkyloxy;
R₃ is amino, amido, sulfonamido, azido, hydroxyl, halogen, cyano, carboxy, C₁₋₁₀ alkoxy, 5-6 membered heterocycle, C₆aryl-C₁₋₁₀ alkyloxy, C₁₋₁₀ alkyl, or SOₙR₁₂ (n=0, 1, 2);
R₄ is selected from hydrogen, halogen, hydroxyl, carboxy, C₁₋₁₀ alkyl, amino, amido, sulfonamide, SOₙR₁₂ (n=0, 1, 2), C₁₋₁₀ alkoxy, C₆₋₁₀ aryl, 5-6 membered heterocycle, or C₅₋₁₀ heteroaryl;
R₁₀ and R₁₁ are each independently selected from hydrogen or C₁₋₁₀ alkyl;
Q is a phenyl optionally substituted, C₁₋₁₀ alkyl, 5-6 membered heterocycle, or C₇₋₁₂aralkyl.

In a further embodiment, there is provided compound of formula I, or a pharmaceutically acceptable salt thereof, wherein:
R₁ is hydrogen or C₁₋₆ alkyl;
R₂ is hydroxyl, C₁₋₆ alkoxy or C₆aryl-C₁₋₆ alkyloxy;
R₃ is amino, azido, hydroxyl, halogen, cyano, carboxy, C₁₋₆ alkoxy, 5-6 membered heterocycle, or C₆aryl-C₁₋₆ alkyloxy;
R₄ is halogen, hydroxyl, carboxy, C₁₋₆ alkyl, C₁₋₆ alkoxy, 5-6 membered heterocycle, or C₆₋₁₀ aryl;
R₁₀ and R₁₁, are each independently selected from hydrogen or C₁₋₆ alkyl;
Q is optionally substituted phenyl.

In still a further embodiment, there is provided compound of formula II: 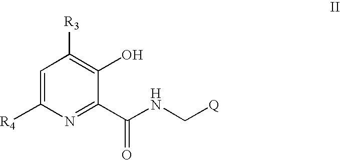
or a pharmaceutically acceptable salt thereof wherein,
R₃ is amino, amido, sulfonamido, azido, hydroxyl, halogen, cyano, carboxy, C₁₋₁₀ alkoxy, 5-6 membered heterocycle, C₆aryl-C₁₋₁₀ alkyloxy, or C₁₋₁₀ alkyl, SOₙR₁₂ (n=0, 1, 2);
R₄ is hydrogen, halogen, hydroxyl, carboxy, C₁₋₁₀ alkyl, amino, amido, sulfonamide, SOₙR₁₂ (n=0, 1, 2), C₁₋₁₀ alkoxy, C₆₋₁₀ aryl, 5-6 membered heterocycle, or C₅₋₁₀ heteroaryl;
Q is optionally substituted phenyl, C₁₋₁₀ alkyl, 5-6 membered heterocycle, or C₇₋₁₂aralkyl.

In still a further embodiment, there is provided compound of formula II, or a pharmaceutically acceptable salt thereof wherein:
R₃ is amino, azido, hydroxyl, halogen, cyano, carboxy, C₁₋₆ alkoxy, 5-6 membered heterocycle, or C₆aryl-C₁₋₆ alkyloxy;
R₄ is hydrogen, halogen, hydroxyl, carboxy, C₁₋₆ alkyl, C₁₋₆ alkoxy, 5-6 membered heterocycle, or C₆₋₁₀ aryl;
Q is optionally substituted phenyl.

In still a further embodiment, there is provided compound of formula III: 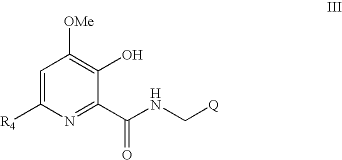
or a pharmaceutically acceptable salt thereof wherein,
R₄ is halogen, hydroxyl, carboxy, C₁₋₁₀ alkyl, amino, amido, sulfonamide, SOₙR₁₂ (n=0, 1, 2), C₁₀ alkoxy, C₆₋₁₀ aryl, 5-6 membered heterocycle, or C₅₋₁₀ heteroaryl;
Q is a phenyl optionally substituted, C₁₋₁₀ alkyl, 5-6 membered heterocycle, or C₇₋₁₂araalkyl.

In still a further embodiment, there is provided compound of formula III, or a pharmaceutically acceptable salt thereof, wherein: R₄ is halogen, hydroxyl, carboxy, C₁₋₆ alkyl, C₁₋₆ alkoxy, 5-6 membered heterocycle, or C₆₋₁₀ aryl; and Q is a phenyl optionally substituted.

In one aspect, the present invention provides novel compounds including:
3′-Hydroxy-[2,4′]bipyridinyl-2′-carboxylic acid 4-fluoro-benzylamide;
3-Hydroxy-4-thiophen-2-yl-pyridine-2-carboxylic acid 4-fluoro-benzylamide;
4-Furan-2-yl-3-hydroxy-pyridine-2-carboxylic acid 4-fluoro-benzylamide;
4-Cyano-3-hydroxy-pyridine-2-carboxylic acid 4-fluoro-benzylamide;
2-(4-Fluoro-benzylcarbamoyl)-3-hydroxy-isonicotinic acid;
6-Bromo-3-hydroxy-4-methoxy-pyridine-2-darboxylic acid 4-fluoro-benzylamide;
6-Bromo-3,4-dihydroxy-pyridine-2-carboxylic acid 4-fluoro-benzylamide;
3-Hydroxy-4-methoxy-6-phenyl-pyridine-2-carboxylic acid 4-fluoro-benzylamide;
3-Hydroxy-4-methoxy-pyridine-2-carboxylic acid 4-fluoro-benzylamide;
6-Bromo-3-hydroxy-4-thiophen-2-yl-pyridine-2-carboxylic acid 4-fluoro-benzylamide;
3,4-Dihydroxy-pyridine-2-carboxylic acid 4-fluoro-benzylamide;
3-Hydroxy-4-methoxy-6-(tetrahydro-furan-2-yl)-pyridine-2-carboxylic acid 4-fluoro-benzylamide;
6-Furan-2-yl-3-hydroxy-4-methoxy-pyridine-2-carboxylic acid 4-fluoro-benzylamide;
4-Bromo-3-hydroxy-6-methoxy-pyridine-2-carboxylic acid 4-fluoro-benzylamide;
4-Bromo-3,6-dihydroxy-pyridine-2-carboxylic acid 4-fluoro-benzylamide;
3-Hydroxy-4-methoxy-6-thiophen-2-yl-pyridine-2-carboxylic acid 4-fluoro-benzylamide;
3-Hydroxy-4-methoxy-6-thiazol-2-yl-pyridine-2-carboxylic acid 4-fluoro-benzylamide;
4,6-Dibromo-3-hydroxy-pyridine-2-carboxylic acid 4-fluoro-benzylamide;
6-(4-Fluoro-benzylamino)-hydroxy-4-methoxy-pyridine-2-carboxylic acid 4-fluoro-benzylamide;
S-Hydroxy-4-methoxy-[2,2′]bipyridinyl-6-carboxylic acid 4-fluoro-benzylamide;
3,4,6-Trimethoxy-pyridine-2-carboxylic acid 4-fluoro-benzylamide;
6-Ethyl-3-hydroxy-4-methoxy-pyridine-2-carboxylic acid 4-fluoro-benzylamide;
3-Hydroxy-4-methoxy-6-vinyl-pyridine-2-carboxylic acid 4-fluoro-benzylamide;
3-Hydroxy-4,6-dimethoxy-pyridine-2-carboxylic acid 4-fluoro-benzylamide;
4-Benzyloxy-6-bromo-3-hydroxy-pyridine-2-carboxylic acid 4-fluoro-benzylamide;
6-(1,2-Dihydroxy-ethyl)-3-hydroxy-4-methoxy-pyridine-2-carboxylic acid 4-fluoro-benzylamide;
4-Azido-3-benzyloxy-6-bromo-pyridine-2-carboxylic acid 4-fluoro-benzylamide;
4-Amino-3-benzyloxy-6-bromo-pyridine-2-carboxylic acid 4-fluoro-benzylamide;
4-Amino-6-bromo-3-hydroxy-pyridine-2-carboxylic acid 4-fluoro-benzylamide;
4,6-Dibromo-3-methoxy-pyridine-2-carboxylic acid 4-fluoro-benzylamide;
3-Hydroxy-6-hydroxymethyl-4-methoxy-pyridine-2-carboxylic acid 4-fluoro-benzylamide;
5′-Hydroxy-4′-methoxy-3,4,5,6-tetrahydro-2H-[1,2′]bipyridinyl-6′-carboxylic acid 4-fluoro-benzylamide;
6-(4-Fluoro-benzylcarbamoyl)-5-hydroxy-4-methoxy-pyridine-2-carboxylic acid;
4-Azido-3-benzyloxy-6-bromo-pyridine-2-carboxylic acid 4-fluoro-benzylamide;
6-(2,2-Dimethyl-[1,3]dioxolan-4-yl)-3-hydroxy-4-methoxy-pyridine-2-carboxylic acid 4-fluoro-benzylamide;
3-Hydroxy-4-methoxy-6-(pyridin-2-ylmethoxy)-pyridine-2-carboxylic acid 4-fluoro-benzylamide;
3-Hydroxy-4-methoxy-6-methoxymethyl-pyridine-2-carboxylic acid 4-fluoro-benzylamide;
3-Hydroxy-4-methoxy-6-(tetrahydro-furan-2-yl)-pyridine-2-carboxylic acid benzylamide;
   and pharmaceutically acceptable salts thereof.

In another aspect, the present invention provides novel compounds including:
3-hydroxy-4-methoxy-6-(tetrahydro-furan-2-yl)-pyridine-2-carboxylic acid [2-(4-fluoro-phenyl)-ethyl]-amide;
3-hydroxy-4-methoxy-6-(tetrahydro-furan-2-yl)-pyridine-2-carboxylic acid (pyridin-2-ylmethyl)-amide;
3-hydroxy-4-methoxy-6-(tetrahydro-furan-2-yl)-pyridine-2-carboxylic acid cyclohexylmethyl-amide;
(+)-3-hydroxy-4-methoxy-6-(tetrahydro-furan-2-yl)-pyridine-2-carboxylic acid 4-fluoro-benzylamide;
(−)-3-hydroxy-4-methoxy-6-(tetrahydro-furan-2-yl)-pyridine-2-carboxylic acid 4-fluoro-benzylamide;
4-acetylamino-3-hydroxy-6-(tetrahydro-furan-2-yl)-pyridine-2-carboxylic acid 4-fluoro-benzylamide;
3-hydroxy-4-methanesulfonyl-6-(tetrahydro-furan-2-yl)-pyridine-2-carboxylic acid 4-fluoro-benzylamide;
6-furan-2-yl-3-hydroxy-4-methylsulfanyl-pyridine-2-carboxylic acid 4-fluoro-benzylamide;
3-hydroxy-6-methoxy-4-vinyl-pyridine-2-carboxylic acid 4-fluoro-benzylamide;
3-hydroxy-4-phenylacetylamino-6-(tetrahydro-furan-2-yl)-pyridine-2-carboxylic acid 4-fluoro-benzylamide;
6-furan-2-yl-3-hydroxy-4-phenylmethanesulfonylamino-pyridine-2-carboxylic acid 4-fluoro-benzylamide;
3-Hydroxy-4-methoxy-6-(tetrahydro-furan-2-yl)-pyridine-2-carboxylic acid 4-methyl-benzylamide;
3-Hydroxy-4-methoxy-6-(tetrahydro-furan-2-yl)-pyridine-2-carboxylic acid 4-methoxy-benzylamide;
3-Hydroxy-4-methoxy-6-(tetrahydro-furan-2-yl)-pyridine-2-carboxylic acid 4-trifluoromethoxy-benzylamide;
3-Hydroxy-4-methoxy-6-(tetrahydro-furan-2-yl)-pyridine-2-carboxylic acid 4-trifluoromethyl-benzylamide;
3-Hydroxy-4-methoxy-6-(tetrahydro-furan-2-yl)-pyridine-2-carboxylic acid 2-fluoro-benzylamide;
3-Hydroxy-4-methoxy-6-(tetrahydro-furan-2-yl)-pyridine-2-carboxylic acid 3-fluoro-benzylamide;
3-Hydroxy-4-methoxy-6-(tetrahydro-furan-2-yl)-pyridine-2-carboxylic acid 2,4-difluoro-benzylamide;
3-Hydroxy-4-methoxy-6-(tetrahydro-furan-2-yl)-pyridine-2-carboxylic acid 3,4-difluoro-benzylamide;
3-hydroxy-4-methoxy-6-(tetrahydro-furan-2-yl)-pyridine-2-carboxylic acid (4-fluoro-benzyl)-methyl-amide;
3-Hydroxy-4-methoxy-6-(tetrahydro-furan-2-yl)-pyridine-2-carboxylic acid [1-(4-fluoro-phenyl)-ethyl]-amide;
3-Hydroxy-4-methoxy-6-(tetrahydro-furan-2-yl)-pyridine-2-carboxylic acid 4-bromo-benzylamide;
3-Hydroxy-4-methoxy-6-(tetrahydro-furan-2-yl)-pyridine-2-carboxylic acid 4-chloro-benzylamide;
6-(1,1-Dioxo-[1,2]-thiazinan-2-yl)-3-hydroxy-4-methoxy-pyridine-2-carboxylic acid 4-fluoro-benzylamide;
3-Hydroxy-4-methoxy-6-(pyridin-2-yl sulfanyl)-pyridine-2-carboxylic acid 4-fluoro-benzylamide;
3-Hydroxy-4-methoxy-6-methylsulfanyl-pyridine-2-carboxylic acid 4-fluoro-benzylamide;
3-Hydroxy-6-methanesulfonyl-4-methoxy pyridine-2-carboxylic acid 4-fluorobenzylamide;
3-Hydroxy-4-methoxy-6-(tetrahydrofuran-3-yl)-pyridine-2-carboxylic acid 4-fluoro-benzylamide;
6-Furan-3-yl-3-hydroxy-4-methoxy-pyridine-2-carboxylic acid 4-fluoro-benzylamide;
6-(4-Benzoyl-piperazin-1-yl)-3-hydroxy-4-methoxy-pyridine-2-carboxylic acid 4-fluoro-benzylamide;
3-Hydroxy-4-methoxy-6-morpholin-4-yl-pyridine-2-carboxylic acid 4-fluoro-benzylamide;
3-Hydroxy-4-methoxy-6-(1,3)-oxathioan-2-yl-pyridine-2-carboxylic acid 4-fluoro-benzylamide;
3-Hydroxy-4-methoxy-6-(5-methyl-(1,3)-oxathioan-2-yl)-pyridine-2-carboxylic acid 4-fluoro-benzylamide;
6-(1,3)-Dioxolan-2-yl-3-hydroxy-4-methoxy-pyridine-2-carboxylic acid 4-fluoro-benzylamide;
3-Hydroxy-4-methoxy-6-(4-methyl-(1,3)dioxolan-2-yl)-pyridine-2-carboxylic acid 4-fluoro-benzylamide;
6-(4-Benzyloxymethyl-(1,3)-dioxolan-2-yl)-3-hydroxy-4-methoxy-pyridine-2-carboxylic acid 4-fluoro-benzylamide;
3-Hydroxy-6-(4-hydroxymethyl-(1,3)-dioxolan-2-yl)-4-methoxy-pyridine-2-carboxylic acid 4-fluoro-benzylamide;
6-(1,3)-Dioxan-2-yl-3-hydroxy-4-methoxy-pyridine-2-carboxylic acid 4-fluoro-benzylamide;
3-Hydroxy-4-methoxy-6-(2-methyl-(1,3)-dioxolan-2-yl)-pyridine-2-carboxylic acid 4-fluoro-benzylamide;
6-(4-Fluoro-benzylcarbamoyl)-3-hydroxy-4-methoxy-pyridine-2-carboxylic acid methyl ester;
3-Hydroxy-4-methoxy-pyridine-2,6-dicarboxylic acid bis-(4-fluoro-benzylamide);
3-Hydroxy-4-methoxy-6-(tetrahydro-furan-2-yl)-pyridine-2-carboxylic acid 4-nitro-benzylamide;
3′-Hydroxy-6′-(tetrahydro-furan-2-yl)-3,4,5,6-tetrahydro-2H-(1,4′)bipyridinyl-2′carboxylic acid 4-fluoro-benzylamide; PS and pharmaceutically acceptable salts thereof.

Reference hereinafter to a compound according to the invention includes compounds of the general formula (I) and their pharmaceutically acceptable salts, hydrates and solvates.

In one embodiment, the compounds of the present invention are the (+) enantiomer having an enantiomeric excess of 99%.

In one embodiment, the compounds of the present invention are the (+) enantiomer having an enantiomeric excess of 95%.

In one embodiment, the compounds of the present invention are the (+) enantiomer having an enantiomeric excess of 90%.

In one embodiment, the compounds of the present invention are the (−) enantiomer having an enantiomeric excess of 99%.

In one embodiment, the compounds of the present invention are the (−) enantiomer having an enantiomeric excess of 95%.

In one embodiment, the compounds of the present invention are the (−) enantiomer having an enantiomeric excess of 90%.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “alkyl” represents a linear, branched or cyclic hydrocarbon moiety having 1 to 10 carbon atoms, which may have one or more double bonds or triple bonds in the chain, and is optionally substituted. Examples include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cyclohexenyl, cyclohexdienyl and cyclohexyl. The term alkyl is also meant to include alkyls in which one or more hydrogen atom is replaced by a halogen, i.e. an alkylhalide. Examples include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, the alkyl groups can also be optionally substituted by, for example, hydroxy, amino, amido, and/or carboxy.

The term “alkoxy” represents an alkyl which is covalently bonded to the adjacent atom through an oxygen atom. Like the alkyl groups, the alkoxy groups can also be optionally substituted. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy and neohexyloxy. The alkoxy groups can be optionally substituted by, for example, halogens, hydroxy, amino, amido, and/or carboxy.

The term “aryl” represents a carbocyclic moiety containing at least one benzenoid-type ring (i.e. may be monocyclic or polycyclic), and which may be optionally substituted with one or more substituents. Examples include but are not limited to phenyl, tolyl, dimethyphenyl, aminophenyl, anilinyl, naphthyl, anthryl, phenanthryl or biphenyl. The alkoxy groups can be optionally substituted by, for example, halogens, hydroxy, amino, amido, and/or carboxy.

The term “aralkyl” represents an aryl group attached to the adjacent atom by a C₁₋₁₀ alkyl. Like the aryl groups, the aralkyl groups can also be optionally substituted. Examples include but are not limited to benzyl, benzhydryl, trityl, phenethyl, 3-phenylpropyl, 2-phenylpropyl, 4-phenylbutyl and naphthylmethyl. The aralkyl groups can be optionally substituted by, for example, halogens, hydroxy, amino, amido, and/or carboxy.

“Aralkyloxy” represents an aralkyl which is covalently bonded to the adjacent atom through an oxygen atom. Like the aryl groups, the aralkyloxy groups can also be optionally substituted. Examples include but are not limited to benzyloxy, benzhydryloxy, trityloxy, phenethyloxy, 3-phenylpropyloxy, 2-phenylpropyloxy, 4-phenylbutyloxy and naphthylmethoxy. The aralkyloxy groups can be optionally substituted by, for example, halogens, hydroxy, amino, amido, and/or carboxy.

The term “acceptable” means that it must not be deleterious to the recipient thereof.

“Halogen atom” is specifically a fluoride atom, chloride atom, bromide atom or iodide atom.

The term “independently” means that a substituent can be the same or a different definition for each item.

The term “amidino” represents —C(═NRₐ)NR_{b}R_{c} wherein Rₐ, R_{b} and R_{c} are each independently selected from H, C₁₋₁₀ alkyl, C₆₋₁₂ aryl or C₆₋₁₂ aralkyl (e.g. C₇₋₁₂ aralkyl), or R_{b} and R_{c} are taken together with the nitrogen to which they are attached to form a 3 to 10 membered heterocycle.

The term “guanidino” represents —N(R_{d})C(═NRₑ)NR_{f}R_{g} wherein R_{d}, Rₑ, R_{f} and R_{g} are each independently selected from H, C₁₀ alkyl, C₆₋₁₂ aryl or C₆₋₁₂ aralkyl (e.g. C₇₋₁₂ aralkyl), or R_{f} and R_{g} are taken together with the nitrogen to which they are attached to form a 3 to 10 membered heterocycle.

The term “amido” represents —CONH₂, —CONHRₕ, —CONRₕRᵢ, —NHCORₕ—NRₕCORᵢ, wherein Rₕ and Rᵢ are each independently selected from C₁₋₁₀ alkyl, C₆₋₁₂ aryl or C₆₋₁₂ aralkyl (e.g. C₇₋₁₂ aralkyl), or Rₕ and Rᵢ are taken together with the nitrogen to which they are attached to form a 3 to 10 membered heterocycle.

The term “amino” represents a derivative of ammonia obtained by substituting one or more hydrogen atom and include —NH₂, —NHRⱼ and —NRⱼRₖ, wherein Rⱼ and Rₖ are each independently selected from C₁₋₁₀ alkyl, C₆₋₁₂ aryl or C₆₋₁₂ aralkyl (e.g. C₇₋₁₂ aralkyl), or Rⱼ and Rₖ are taken together with the nitrogen to which they are attached to form a 3 to 10 membered heterocycle.

The term “sulfonamido” represents —SO₂NH₂, —SO₂NHR_{L}, —SO₂NR_{L}R_{LL}, and —NR_{L}SO₂R_{LL}, wherein R_{L} and R_{LL} are each independently selected from C₁₋₁₀ alkyl, C₆₋₁₂ aryl or C₇₋₁₂ aralkyl, or R_{L} and R_{LL} are taken together with the nitrogen to which they are attached to form a 3 to 10 membered heterocycle.

The term “heterocycle” represents an optionally substituted saturated, unsaturated or aromatic cyclic moiety wherein said cyclic moiety is interrupted by at least one heteroatom selected from oxygen (O), sulfur (S) or nitrogen (N). Heterocycles may be monocyclic or polycyclic rings. Examples include but are not limited to azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl, furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl. The heterocyclic groups can be optionally substituted by, for example, halogens, hydroxy, amino, amido, and/or carboxy.

The term “heteroaryl” represents an optionally substituted aromatic cyclic moiety wherein said cyclic moiety is interrupted by at least one heteroatom selected from oxygen (O), sulfur (S) or nitrogen (N). Heteroaryls may be monocyclic or polycyclic rings. Examples include but are not limited to azepinyl, aziridinyl, azetyl, diazepinyl, dithiadiazinyl, dioxazepinyl, dithiazolyl, furanyl, isooxazolyl, isothiazolyl, imidazolyl, oxadiazolyl, oxiranyl, oxazinyl, oxazolyl, pyrazinyl, pyridazinyl, pyrimidinyl, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl, furoisoxazolyl, imidazothiazolyl, thienoisothiazolyl, thienothiazolyl, imidazopyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl and pyridopyrimidinyl. The heteroaryl groups can be optionally substituted by, for example, halogens, hydroxy, amino, amido, and/or carboxy.

The term “heteroaralkyl” represents an optionally substituted heteroaryl group attached to the adjacent atom by a C₁₋₁₀ alkyl. The heteroaralkyl groups can be optionally substituted by, for example, halogens, hydroxy, amino, amido, and/or carboxy.

The term “urea” represents —N(Rₐₐ)CONR_{bb}R_{cc} wherein Rₐₐ is H or C₁₋₁₀ alkyl and wherein R_{bb} and R_{cc} are each independently selected from the group consisting of H, C₁₋₁₀ alkyl, C₆₋₁₀ aryl, 3-10 membered heterocycle, and C₆₋₁₂ aralkyl (e.g. C₁₋₁₂ aralkyl), or R_{bb} and R_{cc} are taken together with the nitrogen to which they are attached to form a C₃₋₁₀ heterocycle.

The term “optionally substituted” represents one or more halogen, amino, amidino, amido, azido, cyano, guanidino, hydroxyl, nitro, nitroso, urea, OS(O)₂Rₘ (wherein Rₘ is C₁₋₁₀ alkyl, C₆₋₁₀ aryl or 3-10 membered heterocycle), OS(O)₂ORₙ (wherein Rₙ is H, C₁₋₁₀ alkyl, C₆₋₁₀ aryl or 3-10 membered heterocycle), S(O)₂ORₚ (wherein Rₚ is H, C₁₋₁₀ alkyl, C₆₋₁₀ aryl or 3-10 membered heterocycle), S(O)₀₋₂R_{q} (wherein R_{q} is H, C₁₋₁₀ alkyl, C₆₋₁₀ aryl or 3-10 membered heterocycle), OP(O)ORₛORₜ, P(O)ORₛORₜ (wherein Rₛ and Rₜ are each independently H or C₁₋₁₀ alkyl), C₁₋₁₀alkyl, C₆aryl-C₁₋₁₀alkyl, C₆₋₁₀aryl, C₁₋₁₀alkoxy, C₆aryl-C₁₋₁₀alkyloxy, C₆₋₁₀aryloxy, 3-10 membered heterocycle, C(O)Rᵤ (wherein Rᵤ is H, C₁₋₁₀ alkyl, C₆₋₁₀ aryl, C₆₋₁₂ aralkyl or 3-10 membered heterocycle), C(O)ORᵥ (wherein Rᵥ is H, C₁₋₁₀ alkyl, C₆₋₁₀ aryl, C₆aryl-C₁₋₁₀ alkyl or 3-10 membered heterocycle), NRₓC(O)R_{w} (wherein Rₓ is H or C₁₋₁₀ oalkyl and R_{w} is H, C₁₋₁₀ alkyl, C₆₋₁₀ aryl, C₆aryl-C₁₋₁₀alkyl or 3-10 membered heterocycle, or Rₓ and R_{w} are taken together with the atoms to which they are attached to form a 3-10 membered heterocycle) or SO₂NR_{y}R_{z} (wherein R_{y} and R_{z} are each independently H, C₁₋₁₀ alkyl, C₆₋₁₀ aryl, 3-10 membered heterocycle or C₆aryl-C₁₋₁₀alkyl).

There is also provided “enantiomers” of the present invention. It will be appreciated that the compounds in accordance with the present invention can contain a chiral center. The compounds in accordance with the present invention may thus exist in the form of two different optical isomers, that is (+) or (−) enantiomers. All such enantiomers and mixtures thereof, including racemic or other ratio mixtures of individual enantiomers, are included within the scope of the invention. The single enantiomer can be obtained by methods well known to those of ordinary skill in the art, suchas chiral HPLC, enzymatic resolution and chiral auxiliary derivatization.

It will also be appreciated that the compounds in accordance with the present invention can contain more than one chiral centers. The compounds of the present invention may thus exist in the form of different diastereomers. All such diastereomers and mixtures thereof are included within the scope of the invention. The single diastereomer can be obtained by method well known in the art, such as HPLC, crystallization and chromatography.

The optical purity is numerically equivalent to the “enantiomeric excess”. The term “enantiomeric excess” is defined in percentage (%) value as follows: [mole fraction (major enantiomer)−mole fraction (minor enantiomer)]×100. An example of ee of 99% represents a ratio of 99.5% of one enantiomer and 0.5% of the opposite enantiomer.

There is also provided “pharmaceutically acceptable salts” of the compounds of the present invention. The salt(s) must be “acceptable” in the sense of not being deleterious to the recipient thereof. By the term pharmaceutically acceptable salts of compounds are meant those derived from pharmaceutically acceptable inorganic and organic acids and bases. Examples of suitable acids include but are not limited to hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts.

Also meant by “pharmaceutically acceptable salts” are salts derived from appropriate bases include alkali metal, alkaline earth metal or ammonium salts. Non-limiting examples of such salts known by those of ordinary skill include without limitation calcium, potassium, sodium, choline, ethylenediamine, tromethamine, arginine, glycinelycine, lycine, magnesium and meglumine.

There is also provided pharmaceutically acceptable hydrates of the compounds of the present invention. The hydrate(s) must be “acceptable” in the sense of not being deleterious to the recipient thereof. “Hydrates” exist when the compound of the invention incorporates water. The hydrate may contain one or more molecule of water per molecule of compound of the invention. Illustrative non-limiting examples include monohydrate, dihydrate, trihydrate and tetrahydrate. The hydrate may contain one or more molecule of compound of the invention per molecule of water. An illustrative non-limiting example includes semi-hydrate. In one embodiment, the water may be held in the crystal in various ways and thus, the water molecules may occupy lattice positions in the crystal, or they may form bonds with salts of the compounds as described herein. The hydrate must be “acceptable” in the sense of not being deleterious to the recipient thereof. The hydration may be assessed by methods known in the art such as Loss on Drying techniques (LOD) and Karl Fisher titration.

The term “Solvate” means that compound of the invention incorporates one or more pharmaceutically acceptable solvent. The solvate(s) must be “acceptable” in the sense of not being deleterious to the recipient thereof. The solvate may contain one or more molecule of solvent per molecule of compound of the invention or may contain one or more molecule of compound of the invention per molecule of solvent. In one embodiment, the solvent may be held in the crystal in various ways and thus, the solvent molecule may occupy lattice positions in the crystal, or they may form bonds with salts of the compounds as described herein. The solvate(s) must be “acceptable” in the sense of not being deleterious to the recipient thereof. The solvation may be assessed by methods known in the art such as Loss on Drying techniques (LOD).

Polymorphs & pseudopolymorphs: It will be appreciated by those skilled in the art that the compounds in accordance with the present invention can exist in several different crystalline forms due to a different arrangement of molecules in the crystal lattice. This may include solvate or hydrate (also known as pseudopolymorphs) and amorphous forms. All such crystalline forms and polymorphs are included within the scope of the invention. The polymorphs may be characterized by methods well known in the art. Examples of analytical procedures that may be used to determine whether polymorphism occurs include: melting point (including hot-stage microscopy), infrared (not in solution), X-ray powder diffraction, thermal analysis methods (e.g. differential scanning calorimetry (DSC) differential thermal analysis (DTA), thermogravimetric analysis (TGA)), Raman spectroscopy, comparative intrinsic dissolution rate, scanning electron microscopy (SEM).

When there is a sulfur atom present, the sulfur atom can be at different oxidation levels, i.e. S, SO, or SO₂. All such oxidation levels are within the scope of the present invention.

When there is a nitrogen atom present, the nitrogen atom can be at different oxidation levels, i.e. N or NO. All such oxidation levels are within the scope of the present invention.

In one embodiment, there is provided a method of preventing or treating HIV infection in a subject which comprises administering to the subject a therapeutically effective amount of a compound of the present invention.

In one embodiment, there is provided a method of preventing or treating HIV infection in a subject which comprises administering to the subject a therapeutically effective amount of a combination or pharmaceutical composition of the present invention.

In one embodiment, there is provided a method of preventing, delaying or treating AIDS in a subject which comprises administering to the subject a therapeutically effective amount of a compound of the present invention.

In one embodiment, there is provided a method of preventing, delaying or treating AIDS in a subject which comprises administering to the subject a therapeutically effective amount of a combination or a pharmaceutical composition of the present invention.

In one embodiment, there is provided a method of preventing HIV replication in a subject which comprises administering to the subject a therapeutically effective amount of a compound of the present invention.

In one embodiment, there is provided a method of preventing HIV replication in a subject which comprises administering to the subject a therapeutically effective amount of a combination or a pharmaceutical composition of the present invention.

In one embodiment, there is provided a method of inhibiting HIV integrase in a subject which comprises administering to the subject a therapeutically effective amount of a compound of the present invention.

In one embodiment, there is provided a method of inhibiting HIV integrase in a subject which comprises administering to the subject a therapeutically effective amount of a combination or a pharmaceutical composition of the present invention.

In one embodiment, there is provided a method of preventing integration of HIV DNA into host cell DNA in a subject which comprises administering to the subject a therapeutically effective amount of a compound of the present invention.

In one embodiment, there is provided a method of preventing integration of HIV DNA into host cell DNA in a subject which comprises administering to the subject a therapeutically effective amount of a combination or a pharmaceutical composition of the present invention.

In one embodiment, there is provided a method of preventing the 3′-end processing of HIV DNA in a subject which comprises administering to the subject a therapeutically effective amount of a compound of the present invention.

In one embodiment, there is provided a method of preventing the 3′-end processing of HIV DNA in a subject which comprises administering to the subject a combination or a pharmaceutical composition of the present invention.

In one embodiment, there is provided a method of preventing the HIV DNA strand transfer to the host cell DNA in a subject which comprises administering to the subject a therapeutically effective amount of a compound of the present invention.

In one embodiment, there is provided a method of preventing the HIV DNA strand transfer to the host cell DNA in a subject which comprises administering to the subject a therapeutically effective amount of a combination or a pharmaceutical composition of the present invention.

In one embodiment, there is provided a method of preventing, or delaying opportunistic infections in HIV-infected subject which comprises administering to the subject a therapeutically effective amount of a compound of the present invention.

In one embodiment, the opportunistic infection is selected from CMV retinitis, Pneumocystis carinii pneumonia, Mycobacterium avium complex, cryptococcal meningitis, or herpes simplex.

In another embodiment, the invention provides the use of a compound of the present invention for the manufacture of a medicament for preventing or treating HIV infection or preventing, delaying or treating AIDS.

In another embodiment, the invention provides the use of a compound of the present invention for the manufacture of a medicament for preventing or treating HIV infection or preventing, delaying or treating AIDS.

In another embodiment, the invention provides the use of a combination of the invention for the manufacture of a medicament for preventing or treating HIV infection or preventing, delaying or treating AIDS.

In another embodiment, the invention provides the use of a compound of the present invention for the manufacture of a medicament for preventing anyone of HIV replication, integration of HIV DNA into host cell DNA, 3′-end processing of HIV DNA or HIV DNA strand transfer to the host cell DNA.

In another embodiment, the invention provides the use of a combination of the invention for the manufacture of a medicament for preventing anyone of HIV replication, integration of HIV DNA into host cell DNA, 3′-end processing of HIV DNA or HIV DNA strand transfer to the host cell DNA.

In another embodiment, the invention provides the use of a compound of the present invention for the manufacture of a medicament for inhibiting HIV integrase.

In another embodiment, the invention provides the use of a combination of the invention for the manufacture of a medicament for inhibiting HIV integrase.

According to a further embodiment, the subject in the above-mentioned methods and uses is a human.

In another aspect, the present invention provides a combination comprising a therapeutically effective amount of the present invention, and a therapeutically effective amount of at least one antiviral agent.

In another embodiment, the present invention provides a combination comprising a therapeutically effective amount of a compound of the present invention, and a therapeutically effective amount of at least one antiviral agent wherein said antiviral agent is selected from nucleoside and nucleotide analog reverse transcriptase inhibitors, non-nucleoside reverse transcriptase inhibitors, protease inhibitors, attachment and fusion inhibitors, integrase inhibitors or maturation inhibitors.

In another embodiment, the present invention provides a combination comprising a therapeutically effective amount of a compound of the present invention, and a therapeutically effective amount of at least one antiviral agent wherein said antiviral agent is selected from nucleoside and nucleotide analog reverse transcriptase inhibitors, non-nucleoside reverse transcriptase inhibitors or protease inhibitors.

In another embodiment, the present invention provides a combination comprising a therapeutically effective amount of a compound of the present invention, and a therapeutically effective amount of at least one antiviral agent wherein said antiviral agent is nucleoside and nucleotide analog reverse transcriptase inhibitors.

In another embodiment, the present invention provides a combination comprising a therapeutically effective amount of a compound of the present invention, and a therapeutically effective amount of at least one antiviral agent wherein said antiviral agent is non-nucleoside reverse transcriptase inhibitors.

In another embodiment, the present invention provides a combination comprising a therapeutically effective amount of a compound of the present invention, and a therapeutically effective amount of at least one antiviral agent wherein said antiviral agent is protease inhibitors.

In one embodiment, the nucleoside and nucleotide analog reverse transcriptase inhibitors is selected from 3TC (lamivudine, Epivir®), AZT (zidovudine, Retrovir®), Emtricitabine (Coviracil®, formerly FTC), d4T (2′,3′-dideoxy-2′,3′-didehydro-thymidine, stavudine and Zerit®), tenofovir (Viread®), 2′,3′-dideoxyinosine (ddI, didanosine, Videx®), 2′,3′-dideoxycytidine (ddC, zalcitabine, Hivid®), Combivir® (AZT/3TC or zidovudine/lamivudine combination), Trivizir® (AZT/3TC/abacavir or zidovudine/lamivudine/abacavir combination), abacavir (1592U89, Ziagen®), SPD-754, Elvucitabine (ACH-126,443, Beta-L-Fd4C), Alovudine (MIV-310), DAPD (amdoxovir), Racivir, 9-[(2-hydroxymethyl)-1,3-dioxolan-4-yllguanine or 2-amino-9-[(2-hydroxymethyl)-1,3-dioxolan-4-yl]adenine.

In another embodiment, the non-nucleoside reverse transcriptase inhibitor is selected from Nevirapine (Viramune®, NVP, BI-RG-587), delavirdine (Rescriptor®, DLV), efavirenz (DMP 266, Sustiva®), GW5634, GW8248, (+)-Calanolide A, Capravirine (AG1549, formerly S-1153), DPC083, MIV-150, TMC120, TMC125 or BHAP (delavirdine), calanolides or L-697,661 (2-Pyridinone 3benzoxazolMeNH derivative).

In another embodiment, the protease inhibitor is selected from nelfinavir (Viracept®, NFV), amprenavir (141W94, Agenerase®), indinavir (MK-639, IDV, Crixivan®), saquinavir (Invirase®, Fortovase®, SQV), ritonavir (Norvir®, RTV), lopinavir (ABT-378, Koletra®), Atazanavir (BMS232632), mozenavir (DMP-450), fosamprenavir (GW433908), RO033-4649, Tipranavir (PNU-140690), GW640385 (VX-385) or TMC114.

In another embodiment, the attachment and fusion inhibitor is selected from T-20 (enfuvirtide, Fuzeon®), T-1249, Schering C (SCH-C), Schering D (SCH-D), GW873140, FP21399, KRH-2731, PRO-140, PRO542, PRO452, TNX-355, AK602, TAK-220, UK-427,857 or soluble CD4, CD4 fragments, CD4-hybrid molecules, and BMS-488043.

In another embodiment, the integrase inhibitor is selected from S-1360 or L-870,810.

In another embodiment, the maturation inhibitor is PA-457.

In another embodiment, the pharmaceutical antiviral agent is a zinc finger inhibitor and is azodicarbonamide (ADA).

In another embodiment, the antiviral agent is an antisense drug and is HGTV43.

In another embodiment, the antiviral agent is an immunomodulator, immune stimulator or cytokine selected from interleukin-2 (IL-2, Aldesleukin, Proleukin), granulocyte macrophage colony stimulating factor (GM-CSF), erythropoietin, Multikine, Ampligen, thymomodulin, thymopentin, foscarnet, HE2000, Reticulose, Murabutide, Resveratrol, HRG214, HIV-1 Immunogen (Remune) or EP HIV-1090.

In another embodiment, the antiviral agent is selected from 2′,3′-dideoxyadenosine, 3′-deoxythymidine, 2′,3′-dideoxy-2′,3′-didehydrocytidine, ribavirin, acyclovir, ganciclovir; interferons such as alpha-, beta- and gamma-interferon; glucuronation inhibitors such as probenecid; or TIBO drugs, HEPT, TSAO derivatives.

In another embodiment, the present invention provides a combination comprising a therapeutically effective amount of a compound of the present invention, and a therapeutically effective amount of at least one further antiviral agent wherein said compound and said antiviral agent are administered sequentially or simultaneously.

In a further embodiment, said compound and said antiviral agent are administered sequentially.

In a further embodiment, said compound and said antiviral agent are administered simultaneously.

In a further embodiment, said compound and said antiviral agent are administered substantially simultaneously.

In another embodiment, the present invention provides a combination comprising a therapeutically effective amount of a compound of the present invention, and a therapeutically effective amount of at least one further antiviral agent wherein said compound and said antiviral agent are present in a synergistic ratio.

It will be clear to a person of ordinary skill that if a further additional therapeutic agent is required or desired, ratios will be readily adjusted. It will be understood that the scope of combinations described herein is not limited to the antiviral agents listed above, but includes in principles any therapeutic agent useful for the prevention and treatment of HIV infection and AIDS.

The compound and combinations referred to above as well as individual components of such combinations may be administered as pharmaceutical compositions.

A further aspect of the invention is therefore presented as a pharmaceutical composition comprising a compound of the present invention together with at least one pharmaceutically acceptable carrier or excipient thereof.

In another embodiment, the present invention provides a pharmaceutical composition comprising a compound of the present invention or a pharmaceutically acceptable salts, hydrates or solvates thereof or combination as defined herein together with one or more pharmaceutically acceptable carrier or excipient thereof.

The carrier(s) or excipient(s) must be “acceptable” in the sense of being compatible with the other ingredients of the formulation and not being deleterious to the recipient thereof.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, alternatively in the range of 0.5 to 60 mg/kg/day, in a further alternative in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 1 to 1500 mg, as a further example the unit dosage form is containing 10 to 1000 mg, as a further example the unit dosage form is containing 50 to 750 mg of active ingredient per unit dosage form.

Ideally the active ingredient should be administered to achieve peak plasma concentrations of the active compound of from about 1 to about 75 μM, preferably about 2 to 50 μM, most preferably about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

While it is possible that, for use in therapy, a compound or combination of the invention may be administered as the raw chemical it is preferable to present the active ingredient as a pharmaceutical composition.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds and combinations according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis, the compounds and combinations according to the invention may be formulated as ointments, creams or lotions, or as a transdermal patch. Such transdermal patches may contain penetration enhancers such as linalool, carvacrol, thympl, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the compounds and combinations according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds and combinations according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The compounds of the invention have been found to have activity in the inhibition of HIV integrase as described in example 21, generally with an observed inhibitory activity at 50 μM.

Certain compounds of the present invention have also been tested in an assay for HIV activity, as described in Example 22, and generally having an IC₅₀ value of less than 10 μM.

In the foregoing and in the following examples, all temperatures are set forth uncorrected in degrees Celsius; and, unless otherwise indicated, all parts and percentages are by weight.

The entire disclosures of all applications, patents and publications, cited above and below, and of corresponding U.S. Provisional Application No. 60/515,443, filed Oct. 30, 2003 are hereby incorporated by reference.

The following general schemes and examples are provided to illustrate various embodiments of the present invention and shall not be considered as limiting in scope.

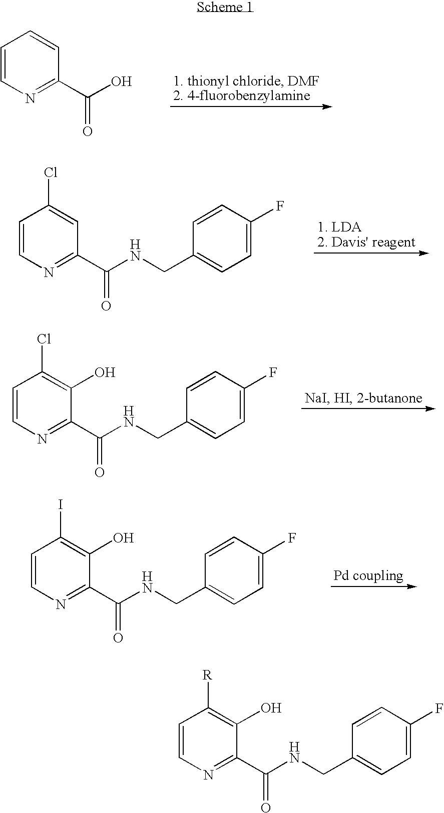

### EXAMPLE 1

### 3-Hydroxy-[2,4′]bipyridinyl-2′-carboxylic acid 4-fluoro-benzylamide compound 1

Step I

### 4-Chloro-pyridine-2-carboxylic acid 4-fluoro-benzylamide

To a solution of picolinic acid (1 g, 8.12 mmol) in thionyl chloride (3 ml) at 45° C. under nitrogen, was added DMF (100 μl) The solution was stirred overnight. Then thionyl chloride was evaporated and co-evaporated with toluene twice. The residue was dissolved into anhydrous CH₂Cl₂ (10 ml), and to the solution was introduced 4-fluorobenzylamine (2.6 g in CH₂Cl₂) slowly at 0° C. The mixture was stirred at room temperature for 3 h. After removal of the solvent under reduced pressure, a brownish solid was obtained. This crude mixture was subjected to silica gel column chromatography eluting with hexane:ethyl acetate (4:1) to afford the desired product in a yield of 1 g.

¹H NMR (400 MHz, CDCl₃): δ [ppm] 8.41 (d, 1H), 8.27 (br s, 1H), 8.21 (s, 1H), 7.42 (d, 1H), 7.31 (m, 2H), 7.00 (m, 2H), 4.61 (d, 2H).

LC/MS: m/z 265.1 (M+H⁺).

Step II

### 4-Chloro-3-hydroxy-pyridine-2-carboxylic acid 4-fluoro-benzylamide

To a solution of diisopropylamine (1.44 ml, 10.3 mmol) in dry THF (10 ml) was added n-butyl lithium (4.1 ml, 2.5 M in hexane) at −78° C. The solution was stirred at this temperature for 20 min. Then a solution of 4-chloro-pyridine-2-carboxylic acid 4-fluoro-benzylamide (972 mg, 3.67 mmol) in dry THF (5 ml) was introduced into the fresh LDA solution at −78° C. The mixture was stirred for 90 min and then to it was added a solution of Davis's reagent (842.7 mg, 3.67 mmol) in dry THF (5 ml). The reaction mixture was agitated overnight and slowly warmed up to rt. This mixture was diluted with ether (100 ml) and washed with water (2×50 ml). The ether layer was dried with anhydrous sodium sulfate, filtered, and evaporated to afford a brownish residue. This crude product was purified on silica gel chromatography using hexane and ethyl acetate (4:1) to obtain a yellowish solid (510 mg).

¹H NMR (400 MHz, CDCl₃): δ [ppm] 8.35 (br s, 1H), 7.92 (d, 1H), 7.42 (d, 1H), 7.31 (m, 2H), 7.04 (m, 2H), 4.60 (d, 2H).

LC/MS: m/z 281.0 (M+H⁺).

Step III

### 4-Iodo-3-hydroxy-pyridine-2-carboxylic acid 4-fluoro-benzylamide

To a solution of 4-chloro-3-hydroxy-pyridine-2-carboxylic acid 4-fluoro-benzylamide (234 mg, 0.83 mmol) in 2-butanone (3 ml), were added NaI (630 mg, 4.15 mmol) and HI (31 μl, 47% in water). The mixture was refluxed for 2·d and then was neutralized with sodium bicarbonate to pH 7. After removal of the solvent, the brown residue was dissolved into ether (100 ml) and washed with sodium bisulfite and water consecutively. The ether layer was dried over anhydrous sodium sulfate, filtered and evaporated under reduced pressure. The mixture was subjected to flash chromatography using hexane and ethyl acetate (9:1) to obtain 230 mg of the desired compound as a yellowish solid.

¹H NMR (400 MHz, CDCl₃): δ [ppm] 8.40 (br s, 1H), 7.81 (d, 1H), 7.65 (d, 1H), 7.32 (m, 2H), 7.04 (m, 2H), 4.60 (d, 2H).

LC/MS: m/z 373.0 (M+H⁺).

Step IV

### 3-Hydroxy-[2,4′]bipyridinyl-2′-carboxylic acid 4-fluoro-benzylamide

To a solution of 4-Iodo-3-hydroxy-pyridine-2-carboxylic acid 4-fluoro-benzylamide (50 mg, 0.13 mmol) in dioxane (2 ml) were added 2-trimethylstannyl-pyridine (64.8 mg, 0.26 mmol) and palladium tetrakistriphenylphosphine (12.4 mg, 0.01 mmol). The mixture was stirred under nitrogen at 100° C. overnight. After removal of dioxane under reduced pressure, the resulting residue was purified on flash chromatography using hexane and ethyl acetate (7:3) to provide 40 mg of the desired product.

¹H NMR (400 MHz, CDCl₃): δ (ppm] 13.20 (s, 1H), 8.76 (d, 1H), 8.53 (br s, 1H), 8.27 (d, 1H), 8.15 (d, 1H), 8.08 (d, 1H), 7.81 (m, 1H), 7.32 (m, 3H), 7.06 (m, 2H), 4.64 (d, 2H).

LC/MS: m/z 323.0 (M+H⁺).

The following compounds were prepared using a similar procedure:

### 3-Hydroxy-4-thiophen-2-yl-pyridine-2-carboxylic acid 4-fluoro-benzylamide compound 2

¹H NMR (400 MHz, CDCl₃): δ [ppm] 13.21 (s, 1H), 8.45 (br s, 1H), 8.01 (d, 1H), 7.86 (m, 1H), 7.64 (d, 1H), 7.48 (m, 1H), 7.34 (m, 2H), 7.16 (m, 1H), 7.05 (m, 2H), 4.62 (d, 2H).

LC/MS: m/z 329.0 (M+H⁺).

### 4-Furan-2-yl-3-hydroxy-pyridine-2-carboxylic acid 4-fluoro-benzylamide compound 3

¹H NMR (400 MHz, CDCl₃): δ [ppm] 13.01 (s, 1H), 8.44 (br s, 1H), 8.04 (d, 1H), 7.78 (m, 1H), 7.54 (d, 1H) 7.35 (m, 3H), 7.05 (m, 2H), 6.57 (m, 1H), 4.62 (d, 2H).

LC/MS: m/z 313.0 (M+H⁺).

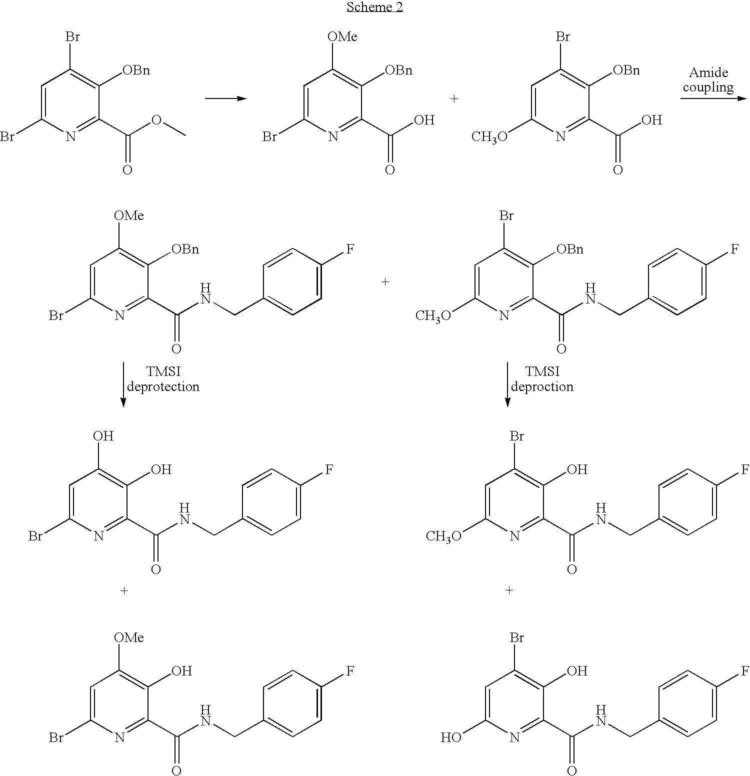

### EXAMPLE 2

### 6-Bromo-3,4-dihydroxy-pyridine-2-carboxylic acid 4-fluorobenzylamide compound 7

Step I

### 3-Benzyloxy-6-bromo-4-methoxy-pyridine-2-carboxylic acid 4-fluorobenzylamide

Starting from the known 3-benzyloxy-4,6-dibromo-pyridine-2-carboxylic methyl ester, compound 3-benzyloxy-6-bromo-4-methoxy-pyridine-2-carboxylic acid was prepared using a procedure described in Ricks, M. J. et al. WO 01/05769 A2. To a solution of this free acid (410 mg, 1.21 mmol) in DMF (910 ml) were added 4-fluorobenzylamine (210 μl, 1.81 mmol), DIPEA (316 μl, 1.81 mmol), and HATU (691 mg, 1.81 mmol). The reaction mixture was stirred at rt for 12 h. Then it was diluted with ether (100 ml) and washed with water (2×50 ml). The organic layer was dried over anhydrous sodium sulfate, filtered and evaporated. The residue was subjected to flash chromatography using hexane and ethyl acetate (6:4) to provide 450 mg of the title compound as white solid.

Step II

### 6-Bromo-3,4-dihydroxy-pyridine-2-carboxylic acid 4-fluorobenzylamide

A solution of 3-benzyloxy-6-bromo-4-methoxy-pyridine-2-carboxylic acid 4-fluorobenzylamide (100 mg, 0.22 mmol) and trimethylsilyl iodide (160 μl, 1.1 mmol) in dry acetonitrile (3 ml) was stirred under nitrogen at rt for 2 days. Then the solvent was evaporated and co-evaporated one more time with methanol. The residue was dissolved into ether (50 ml) and washed 20% NaHSO₃ (10 ml) and water (20 ml) consecutively. The organic layer was dried over anhydrous sodium sulfate, filtered and evaporated. The crude mixture was purified on preparative TLC using dichloromethane and methanol (9:1) as a developing solvent to afford 25 mg of the title compound.

¹H NMR (400 MHz, CD₃OD): δ [ppm] 7.27 (br s, 2H), 6.94 (m, 3H), 4.80 (br s, 2H).

LC/MS: m/z 341.0 (M+H⁺).

### 6-Bromo-3-hydroxy-4-methoxy-pyridine-2-carboxylic acid 4-fluorobenzylamide compound 6

This compound was isolated from the above-mentioned reaction in a yield of 25 mg.

¹H NMR (400 MHz, CDCl₃): δ [ppm] 12.40 (s, 1H), 8.10 (br s, 1H), 7.32 (m, 2H), 7.04 (m, 2H), 6.96 (s, 1H), 4.57 (d, 2H), 3.94 (s, 3H).

LC/MS: m/z 355.0 (M+H⁺).

### 4-Bromo-3-hydroxy-6-methoxy-pyridine-2-carboxylic acid 4-fluorobenzylamide compound 14

This compound was prepared from 3-benzyloxy-4-bromo-6-methoxy-pyridine-2-carboxylic acid 4-fluorobenzylamide in the same manner as mentioned in step II.

¹H NMR (400 MHz, CDCl₃): δ [ppm] 12.44 (s, 1H), 8.05 (br s, 1H), 7.32 (m, 2H), 7.17 (s, 1H), 7.07 (m, 2H), 4.61 (d, 2H), 3.84 (s, 3H).

LC/MS: m/z 356.8 (M+H⁺).

### 4-Bromo-3,6-dihydroxy-pyridine-2-carboxylic acid 4-fluorobenzylamide compound 15

This compound was isolated from the above-mentioned reaction.

¹H NMR (400 MHz, CDCl₃): δ [ppm] 12.44 (s, 1H), 7.95 (br s, 1H), 7.32 (m, 2H), 7.15 (s, 1H), 7.07 (m, 2H), 4.58 (d, 2H).

### EXAMPLE 3

### 3-Hydroxy-4-methoxy-pyridine-2-carboxylic acid 4-fluorobenzyl-amide compound 9

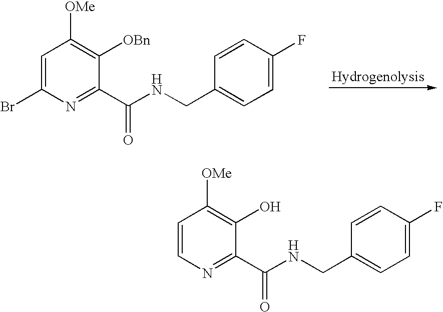

3-benzyloxy-4-methoxy-6-bromo-pyridine-2-carboxylic acid 4-fluorobenzylamide was dissolved into a mixture of methanol and ethyl acetate. To the solution was added a catalytic amount of 10% Pd—C. The flask was attached to a hydrogen balloon and the reaction was run at rt for 1 hr. The mixture was filtered through a pad of celite. Removal of the solvent under reduced pressure afforded the desired compound.

¹H NMR (400 MHz, CDCl₃): δ [ppm] 12.44 (s, 1H), 8.80 (br s, 1H), 8.16 (d, 1H), 7.40 (m, 2H), 7.17 (d, 1H), 7.10 (m, 2H), 4.61 (d, 2H), 4.11 (s, 3H).

LC/MS: m/z 277.0 (M+H⁺).

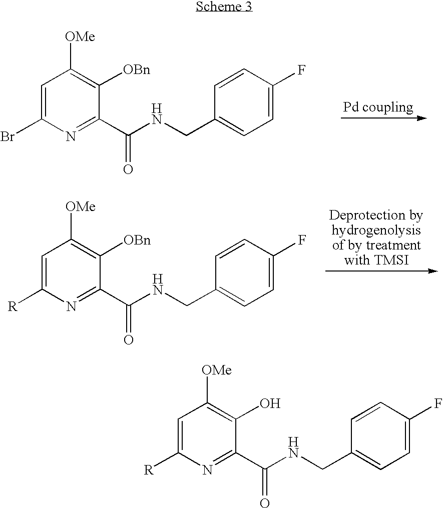

### EXAMPLE 4

### 3-Hydroxy-4-methoxy-6-phenyl-pyridine-carboxylic acid 4-fluorobenzylamide compound 8

A solution of 3-benzyloxy-6-bromo-4-methoxy-pyridine-2-carboxylic acid 4-fluorobenzylamide (71 mg, 0.16 mmol) in a mixture of DME/20% Na₂CO₃ (2 ml/2 ml) were added phenylboronic acid (38.8 mg, 0.32 mmol) and Pd(PPh₃)₄ (11 mg, 0.016 mmol). The reaction mixture was refluxed under nitrogen overnight. The mixture was neutralized to pH 3 and diluted with ether (50 ml). After partition, the organic layer was dried with anhydrous sodium sulfate, filtered. After evaporation of the solvent, the residue was purified on silica gel column using hexane and ethyl acetate (8:2) to provide 30 mg of compound that was deprotected using TMSI in a manner as described in Example 2.

¹H NMR (400 MHz, CDCl₃): δ [ppm] 12.38(s, 1H), 8.50 (br s, 1H), 7.77 (m, 2H), 7.35 (m, 5H), 7.21 (s, 1H), 6.96 (m, 2H), 4.55 (d, 2H), 3.95 (s, 3H).

LC/MS: m/z 353.2 (M+H⁺).

### EXAMPLE 5

### 3-Hydroxy-4-methoxy-6-thiophen-2-yl-pyridine-2-carboxylic acid 4-fluorobenzylamide compound 16

To a solution of 3-benzyloxy-6-bromo-4-methoxy-pyridine-2-carboxylic acid 4-fluorobenzylamide (44.5 mg, 0.1 mmol) in dioxane (4 ml) were added tributyl-thiophen-2-yl stannane (47 μl, 0.15 mmol), and Pd(PPh₃)₄ (12 mg, 0.01 mmol). Under nitrogen, the mixture was stirred at 80° C. overnight. After removal of the solvent, the residue was purified on silica gel column chromatography using hexane and ethyl acetate (4:1) to provide 50 mg of 3-benzyloxy-4-methoxy-6-thiophen-2-yl-pyridine-2-carboxylic acid 4-fluorobenzylamide. This product was further deprotected by using TMSI in a manner as described in Example 2 to obtain the title compound.

¹H NMR (400 MHz, CDCl₃): δ [ppm] 12.30 (s, 1H), 8.35 (br s, 1H), 7.43 (br s, 1H), 7.33 (m, 3H), 7.20 (s, 1H), 7.07 (m, 3H), 4.63 (d, 2H), 4.01 (s, 3H).

LC/MS: m/z 359.1 (M+H⁺).

The following compounds were prepared in a similar manner:

### 3-Hydroxy-4-methoxy-6-thiazol-2-yl-pyridine-2-carboxylic acid 4-fluorobenzylamide compound 17

¹H NMR (400 MHz, CDCl₃): δ [ppm] 12.39 (s, 1H), 8.21 (br s, 1H), 7.78 (d, 1H), 7.3S (s, 1H), 7.29 (m, 3H), 7.01 (m, 2H), 4.58 (d, 2H), 3.97 (s, 3H).

LC/MS: m/z 360.1 (M+H⁺).

### 5-Hydroxy-4-methoxy-[2,2′)bipyridinyl-6-carboxylic acid 4-fluoro-benzylamide compound 20

¹H NMR (400 MHz, CDCl₃): δ [ppm] 12.50 (s, 1H), 8.62 (d, 1H), 8.45 (br s, 1H), 8.26 (d, 1H), 8.08 (s, 1H), 7.94 (t, 1H), 7.37 (m, 2H), 7.29 (m, 1H), 7.06 (m, 2H), 4.57 (d, 2H), 4.07 (s, 3H).

LC/MS: m/z 354.0 (M+H⁺).

### 6-(4-Fluoro-benzylamino)-3-hydroxy-4-methoxy-pyridine-2-carboxylic acid 4-fluoro-benzylamide compound 19

¹H NMR (400 MHz, CDCl₃): δ [ppm] 11.68 (s, 1H), 7.95 (br s, 1H), 7.26 (m, 4H), 7.03 (m, 2H), 6.92 (m, 2H), 6.07 (s, 1H), 4.53 (d, 2H), 4.39 (s, 2H), 3.85 (s, 3H).

LC/MS: m/z 400.0 (M+H⁺).

### 6-Furan-2-yl-3-hydroxy-4-methoxy-pyridine-2-carboxylic acid 4-fluoro-benzylamide compound 13

¹H NMR (400 MHz, CDCl₃): δ [ppm] 12.19 (s, 1H), 8.29 (br s, 1H), 7.47 (m, 1H), 7.33 (m, 3H), 7.07 (m, 2H), 6.89 (m, 1H), 6.48 (m, 1H), 4.61 (d, 2H), 4.01 (s, 3H).

LC/MS: m/z 343.0 (M+H⁺).

### 3-Hydroxy-4-methoxy-6-(tetrahydro-furan-yl)-pyridine-2-carboxylic acid 4-fluoro-benzylamide compound 12

This compound was prepared from 6-furan-2-yl-3-hydroxy-4-methoxy-pyridine-2-carboxylic acid 4-fluorobenzylamide using hydrogenolysis in the presence of a drop of acidic acid.

¹H NMR (400 MHz, CDCl₃): δ [ppm] 12.19 (s, 1H), 8.29 (br s, 1H), 7.31 (m, 2H), 7.07 (m, 3H), 4.82 (m, 1H), 4.59 (m, 2H), 4.05 (m, 1H), 3.95 (m, 4H), 2.25 (m, 1H), 1.95 (m, 2H).

LC/MS: m/z 347.0 (M+H⁺).

Additional compounds were also prepared in a similar manner:

### 6-Furan-3-yl-3-hydroxy-4-methoxy-pyridine-2-carboxylic acid 4-fluoro-benzylamide compound 67

¹H NMR (400 MHz, CDCl₃): δ [ppm] 12.34 (s, 1H), 8.39 (br s, 1H), 7.87 (s, 1H), 7.45 (d, 1H), 7.33 (m, 2H), 7.03 (m, 3H), 6.79 (d, 1H), 4.60 (d, 2H), 3.97 (s, 3H).

LC/MS: m/z 343.1 (M+H⁺).

### 3-Hydroxy-4-methoxy-6-(tetrahydrofuran-3-yl)-pyridine-2-carboxylic acid 4-fluoro-benzylamide compound 66

¹H NMR (400 MHz, CDCl₃): δ [ppm] 12.19 (s, 1H), 8.34 (br s, 1H), 7.33 (m, 2H), 7.04 (m, 2H), 6.77 (s, 1H), 4.58 (d, 2H), 4.03 (m, 2H), 3.92 (s, 3H), 3.86 (m, 2H), 3.45 (m, 1H), 2.30 (m, 1H), 2.10 (m, 1H).

LC/MS: m/z 346.4 (M+H⁺).

### 3-Hydroxy-4-methoxy-6-morpholin-4-yl-pyridine-2-carboxylic acid 4-fluoro-benzylamide 69

The title compound was prepared similarly using a palladium catalyzed coupling C—N protocol, followed a hydrogenolysis using PtO₂ as the catalyst.

¹H NMR (400 MHz, CDCl₃): δ [ppm] 12.81 (s, 1H), 8.10 (br s, 1H), 7.33 (m, 2H), 7.04 (m, 2H), 6.35 (s, 1H), 4.58 (d, 2H), 3.92 (s, 3H), 3.81 (t, 4H), 3.33 (t, 4H).

LC/MS: m/z 362.2 (M+H⁺).

### 6-(4-Benzoyl-piperazin-1-yl)-3-hydroxy-4-methoxy-pyridine-2-carboxylic acid 4-fluoro-benzylamide compound 68

The compound was prepared in similar manner.

¹H NMR (400 MHz, CDCl₃): δ [ppm] 12.82 (s, 1H), 8.05 (br s, 1H), 7.43 (m, 5H), 7.28 (m, 2H), 7.04 (m, 2H), 6.38 (s, 1H), 4.58 (d, 2H), 3.92 (m, 5H), 3.55 (m, 2H), 3.4.0 (m, 4H).

LC/MS: m/z 465.2 (M+H⁺).

### 6-(1,1-Dioxo-[1,2]-thiazinan-2-yl)-3-hydroxy-4-methoxy-pyridine-2-carboxylic acid 4-fluoro-benzylamide compound 62

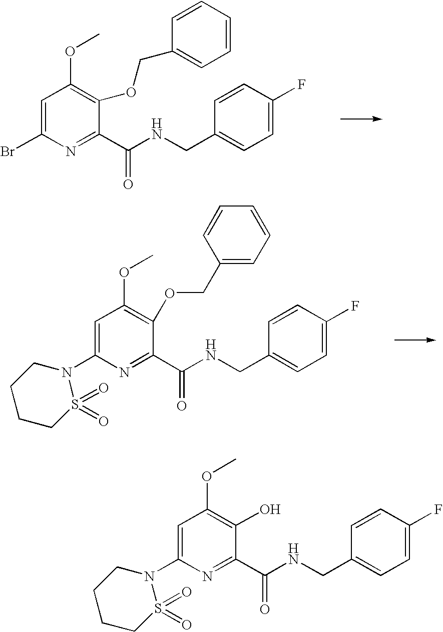

To a solution of 3-benzyloxy-6-bromo-4-methoxy-pyridine-2-carboxylic acid 4-fluoro-benzylamide (44.5 mg, 0.1 mmol) in toluene (4 ml) were added 1,4-butanesultam (16.2 mg, 0.12 mmol), cesium carbonate (65 mg, 0.2 mmol), CuI (1.9 mg, 0.01 mmol), 1,10-phenanthroline (3.6 mg, 0.02 mmol). Under nitrogen, the mixture was stirred at 100° C. overnight. After removal of the solvent under reduced pressure, the residue was dissolved into water (10 mL) and extracted with dichloromethane (3×10 mL), and the combined organic layers were dried over anhydrous sodium sulfate. Evaporation of the solvent under reduced pressure provided a residue, which was purified on silica gel column eluting with hexane and ethyl acetate (5:5) to afford a white solid (45 mg). This product (40 mg) was deprotected using hydrogenolysis in methanol to provide the title compound (30 mg).

¹H NMR (400 MHz, CDCl₃): δ [ppm] 12.22 (s, 1H), 7.90 (br s, 1H), 7.30 (m, 2H), 7.07 (m, 3H), 4.58 (d, 2H), 3.92 (m, 5H), 3.13 (m, 2H), 2.30 (m, 2H), 1.89 (m, 2H).

LC/MS: m/z 410.2 (M+H⁺).

### 3-Hydroxy-4-methoxy-6-(pyridin-2-yl-sulfanyl)-pyridine-2-carboxylic acid 4-fluoro-benzylamide compound 63

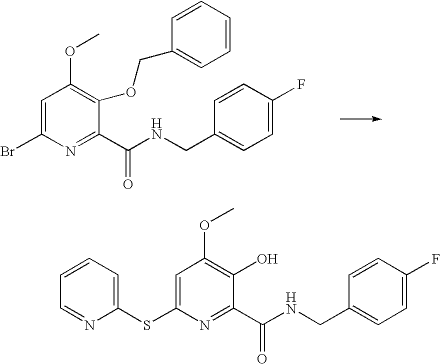

To a solution of 3-benzyloxy-6-bromo-4-methoxy-pyridine-2-carboxylic acid 4-fluoro-benzylamide (44.5 mg, 0.1 mmol) in toluene (4 ml) were added pyridin-2-thiol (16.2 mg, 0.14 mmol), t-BuOK (16.5 mg, 0.14 mmol), Pd₂ dba₃ (5 mg, 5 mol %), Xanphos (5.8 mg₁ 10 mol %). Under nitrogen, the mixture was stirred at 100° C. for 12 h. After removal of the solvent under reduced pressure, the residue was dissolved into 10 ml of water and extracted with dichloromethane (3×10 mL), and the combined organic layers were dried over anhydrous sodium sulfate. Evaporation of the solvent under reduced pressure provided a residue, which was purified by preparative TLC using hexane and ethyl acetate (4:6) as the mobile phase to yield the desired compound as an off-white solid (25 mg).

¹H NMR (400 MHz, CDCl₃): δ [ppm] 12.37(s, 1H), 8.45 (5, H), 8.10 (br 5, 1H), 7.49 (m, 1H), 7.24 (m, 3H), 7.01 (m, 4H), 4.56 (d, 2H), 3.89 (5, 3H).

LC/MS: m/z 386.0 (M+H⁺).

### 3-Hydroxy-4-methoxy-6-methylsulfanyl-pyridine-2-carboxylic acid 4-fluoro-benzylamide compound 64

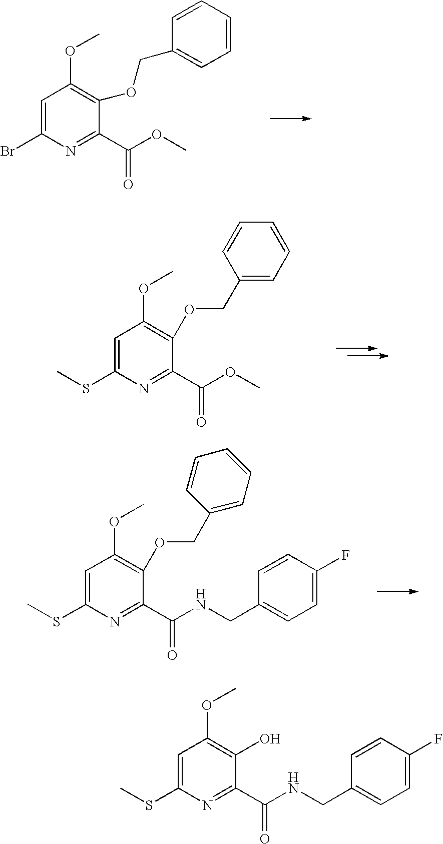 Step I

The mixture of 3-bromo-4-methoxy-2-carboxylic acid methyl ester (50 mg, 0.14 mmol), NaSMe (15 mg, 0.21 mmol), Pd₂ dba₃.CHCl₃ (7.3 mg, 5 mol %) and Xanphos (8.2 mg, 10 mol %) in toluene (5 mL) was heated to 100° C. under nitrogen for 24 h. After removal of the solvent under reduced temperature, the residue was dissolved into 10 mL of water and then extracted with dichloromethane (3×10 mL). The combined organic layers were dried over anhydrous sodium sulfate. Evaporation of the solvent provided a residue, which was purified on silica gel column eluting with hexane and ethyl acetate (7:3) to afford the desired compound 3-benzyloxy-4-methoxy-6-methylsulfanyl-pyridine-2-carboxylic acid methyl ester as a white solid (35 mg).

¹H NMR (400 MHz, CDCl₃): δ [ppm] 7.44 (m, 2H), 7.35 (m, 3H), 6.90 (s, 1H), 5.02 (s, 2H), 3.91 (s, 3H), 3.89 (s, 3H), 2.58 (s, 3H).

Step II

3-benzyloxy-4-methoxy-6-methylsulfanyl-pyridine-2-carboxylic acid methyl ester was hydrolyzed in methanol using sodium hydroxide to provide its corresponding acid, which was coupled with 4-fluorobenzylamine in the presence HATU to give 3-benzyloxy-4-methoxy-6-methylsulfanyl-pyridine-2-carboxylic acid 4-fluoro-benzylamide.

¹H NMR (400 MHz, CDCl₃): δ [ppm] 7.90 (br s, 1H), 7.54 (m, 2H), 7.35 (m, 5H), 7.00 (m, 2H), 6.88 (s, 1H), 5.08 (s, 2H), 4.58 (d, 2H), 3.86 (s, 3H), 2.49 (s, 3H).

Step III

3-benzyloxy-4-methoxy-6-methylsulfanyl-pyridine-2-carboxylic acid 4-fluoro-benzylamide was then debenzylated using TMSI to generate the title compound.

¹H NMR (400 MHz, CDCl₃): δ [ppm] 12.15 (s, 1H), 8.19 (br s, 1H), 7.33 (m, 2H), 7.05 (m, 2H), 6.74 (s, 1H), 4.61 (d, 2H), 3.93 (s, 3H), 2.50 (s, 3H).

LC/MS: m/z 323.1 (M+H⁺).

### 3-Hydroxy-6-methanesulfonyl-4-methoxy pyridine-2-carboxylic acid 4-fluoro-benzylamide compound 65

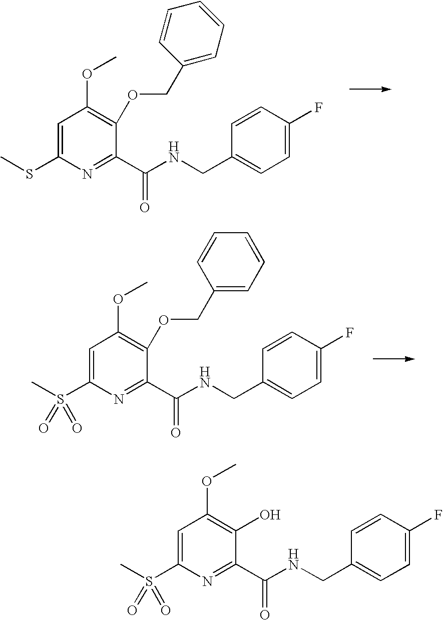

To a solution of 3-benzyloxy-4-methoxy-6-methylsulfanyl-pyridine-2-carboxylic acid 4-fluoro-benzylamide (30 mg, 0.07 mmol) in chloroform (5 mL) was added m-chloroperbenzoic acid (49 mg, 0.21 mmol). The reaction mixture was stirred at rt for 5 h and then treated with 20% NaHSO₃ (1 mL). After stirring for 20 min, the mixture was diluted with water (10 mL) and extracted with chloroform (3×10 mL). The combined organic layers were dried over anhydrous sodium sulfate. After removal of the solvent under reduced pressure, the residue was purified on silica gel column eluting with hexane and ethyl acetate (5:5) to provide a white solid (26 mg), which was further deprotected using hydrogenolysis to yield the title compound as a white solid (18 mg).

¹H NMR (400 MHz, CDCl₃): δ [ppm] 13.02 (s, 1H), 8.20 (br s, 1H), 7.63 (s, 1H), 7.33 (m, 2H), 7.05 (m, 2H), 4.62 (d, 2H), 4.05 (s, 3H), 3.15 (s, 3H).

LC/MS: m/z 355.0 (M+H⁺).

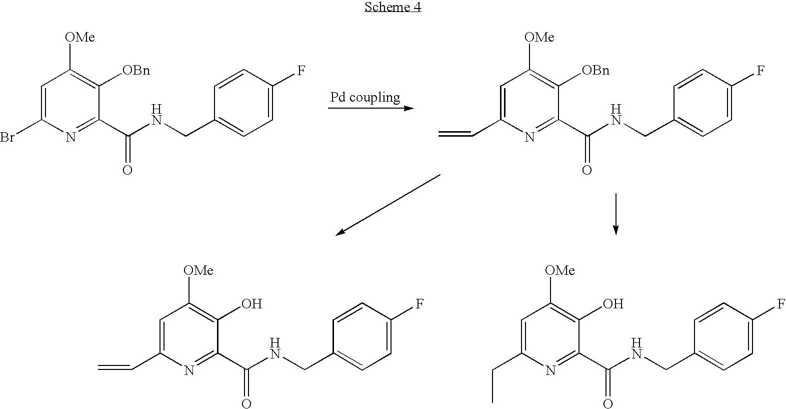

### EXAMPLE 6

### 3-Hydroxy-4-methoxy-6-vinyl-pyridine-2-carboxylic acid 4-fluoro-benzylamide compound 23

The precursor 4-benzyloxy-3-hydroxy-6-vinyl-pyridine-2-carboxylic acid 4-fluoro-benzylamide was prepared by using Pd(PPh₃)₂Cl₂ as a catalyst and refluxing in THF as described in example 4 and was deprotected by using TMSI in a manner as described in example 2.

¹H NMR (400 MHz, CDCl₃): δ [ppm] 12.35 (s, 1H), 8.42 (br s, 1H), 7.34 (m, 2H), 7.02 (m, 3H), 6.69 (q, 1H), 5.97 (m, 1H), 5.41 (m, 1H), 4.61 (d, 2H), 3.97 (s, 3H).

LC/MS: m/z 303.0 (M+H⁺).

### 6-Ethyl-3-hydroxy-4-methoxy-6-pyridine-2-carboxylic acid 4-fluoro-benzylamide compound 22

This compound was prepared from the previous precursor by using hydrogenolysis as described herein.

¹H NMR (400 MHz, CDCl₃): δ [ppm] 12.10 (s, 1H), 8.42 (br s, 1H), 7.34 (m, 2H), 7.02 (m, 3H), 6.69 (s, 1H), 4.61 (d, 2H), 3.92 (s, 3H), 2.67 (m, 2H), 1.24 (t, 3H).

LC/MS: m/z 305.0 (M+H⁺).

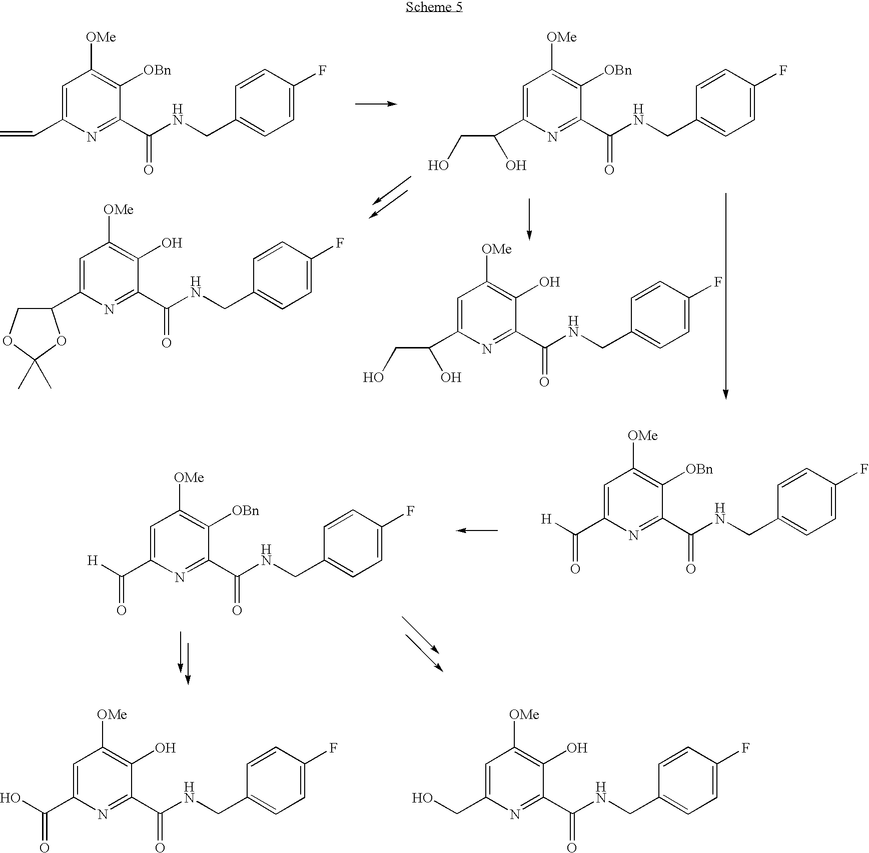

### EXAMPLE 7

### 6-(1,2-Dihydroxy-ethyl)-3-hydroxy-4-methoxy-2-carboxylic acid 4-fluoro-benzylamide compound 26

To a solution of 4-benzyloxy-3-hydroxy-6-vinyl-pyridine-2-carboxylic acid 4-fluoro-benzylamide (39.2 mg, 0.1 mmol) in a mixture of THF and water (2.5 ml:0.5 ml) was added osmium tetroxide (2.5 mg, 0.01 mmol). The mixture was stirred at rt. After its color was changed to dark, 4-methyl morpholine N-oxide (35 mg, 0.03 mmol) was added to the solution. After stirring overnight, 20% NaHSO₃ (1 ml) was added to the mixture. The reaction mixture was diluted with water and extracted with chloroform (3×10 ml). The organic layers were combined together, dried over anhydrous sodium sulfate, filtered. After removal of the solvent, the crude was subjected to preparative TLC to yield 35 mg of 3-benzyloxy-6-(1,2-dihydroxy-ethyl)-4-methoxy-2-carboxylic acid 4-fluoro-benzylamide. This compound (15 mg) was subjected to hydrogenolysis to provide 12 mg of the desired product.

¹H NMR (400 MHz, CD₃OD): δ [ppm] 7.34 (m, 2H), 7.15 (s, 1H), 7.02 (m, 2H), 4.69 (m, 1H), 4.56 (s, 2H), 3.92 (s, 3H), 3.72 (m, 2H).

LC/MS: m/z 427.1 (M+H⁺).

### EXAMPLE 8

### 3-Hydroxy-6-hydroxymethyl-4-methoxy-pyridine-2-carboxylic acid 4-fluoro-benzylamide compound 31

To a solution of 3-benzyloxy-6-(1,2-dihydroxy-ethyl)-4-methoxy-2-carboxylic acid 4-fluoro-benzylamide (18 mg, 0.042 mmol) in a mixture of dioxane and water (2 ml:0.5 ml) was added NaIO₄ (9 mg, 0.042 mmol). The mixture was stirred at rt for 3 h. Then it was diluted with water (20 ml) and extracted with CHCl₃ (3×10 ml). The organic layers were combined together, dried over anhydrous sodium sulfate, filtered. After removal of the solvent, the residue was purified on preparative TLC to provide 14 mg of the corresponding aldehyde, which was further reduced by using NaBH₄ using standard conditions described in the literature to afford 3-benzyloxy-6-hydroxymethyl-4-methoxy-2-carboxylic acid 4-fluoro-benzylamide. This compound was subjected to hydrogenolysis as described in example 3 to provide the desired product.

¹H NMR (400 MHz, CDCl₃): δ [ppm] 12.25 (s, 1H), 8.35 (br s, 1H), 7.34 (m, 2H), 7.02 (m, 2H), 6.87 (s, 1H), 4.62 (s, 2H), 4.60(s, 2H), 3.92 (s, 3H).

### EXAMPLE 9

### 6-(4-Fluoro-benzylcarbamoyl)-5-hydroxy-4-methoxy-pyridine-2-carboxylic acid compound 33

To a solution of the aldehyde in example 8 (40 mg, 0.1 mmol) in a mixture of water (2 ml), t-BuOH (2 ml), and iso-2-butene (0.5 ml) were added NaClO₂ (87 mg), NaH₂PO₄ (87 mg). After stirring at rt for 3 h, the mixture was neutralized to pH 2, diluted with water (20 ml), and extracted CHCl₃ (3×10 ml). The organic layers were combined together, dried over anhydrous sodium sulfate, and filtered. Removal of the solvent under reduced pressure afforded 35 mg of 5-benzyloxy-6-(4-fluoro-benzylcarbamoyl)-4-methoxy-pyridine-2-carbozylic acid. This compound was subjected to hydrogenolysis as described in example 3 to provide the desired product.

¹H NMR (400 MHz, CDCl₃): δ [ppm] 13.15 (br s, 1H), 8.70 (br s, 1H), 7.34 (m, 3H), 7.02 (m, 2H), 4.62 (br s, 2H), 3.92 (s, 3H).

LC/MS: m/z 321.1 (M+H⁺).

### EXAMPLE 10

### 6-(2,2-Dimethyl-(1,3]dioxolan-4-yl)-3-hydroxy-4-methoxy-pyridine-2-carboxylic acid 4-fluoro-benzylamide compound 35

To a solution of 3-benzyloxy-6-(1,2-dihydroxy-ethyl)-4-methoxy-2-carboxylic acid 4-fluoro-benzylamide (21 mg, 0.05 mmol) in CH₂Cl₂ (2.5 ml) was added 2,2-dimethoxypropane (0.4 ml) and 10-camphorsulfonic acid (0.7 mg, 5 mol %). The mixture was stirred at rt for 5 h. After removal of the solvent, the crude mixture was purified on preparative TLC to afford 9.5 mg of 3-benzyloxy-6-(2,2-dimethyl-[1,3]dioxolan-4-yl)-4-methoxy-pyridine-2-carboxylic acid 4-fluoro-benzylamide, which was subjected to hydrogenolysis as described in example 3 to provide the desired product.

¹H NMR (400 MHz, CDCl₃): δ [ppm] 12.25 (s, 1H), 8.70 (br s, 1H), 7.34 (m, 2H), 7.12 (s, 1H), 7.02 (m, 2H), 5.08 (m, 1H), 4.60 (m, 2H), 4.35 (m, 1H), 3.97 (s, 3H), 3.97 (m, 1H), 1.52 (s, 3H), 1.47 (s, 3H).

LC/MS: m/z 377.1 (M+H⁺).

### Cis-3-Hydroxy-4-methoxy-6-(2-methyl-(1,3)-dioxolan-2-yl)-pyridine-2-carboxylic acid 4-fluoro-benzylamide compound 77

The title compound was prepared in a similar manner and was obtained as a 2:1 mixture of cis:trans isomers, which were separated by chromatography.

¹H NMR (400 MHz, CDCl₃): δ [ppm] 12.28 (s, 1H), 8.20 (br s, 1H), 7.31 (m, 2H), 7.25 (s, 1H), 7.05 (m, 2H), 5.29 (m, 1H), 5.05 (m, 1H), 4.62 (m, 2H), 4.45 (m, 1H), 3.95 (S, 3H), 3.88 (m, 1H), 1.44 (d, 3H).

LC/MS: m/z 363.1 (M+H⁺).

### 3-Hydroxy-4-methoxy-6-(1,3)-oxathiolan-2-yl-pyridine-2-carboxylic acid 4-fluoro-benzylamide compound 70

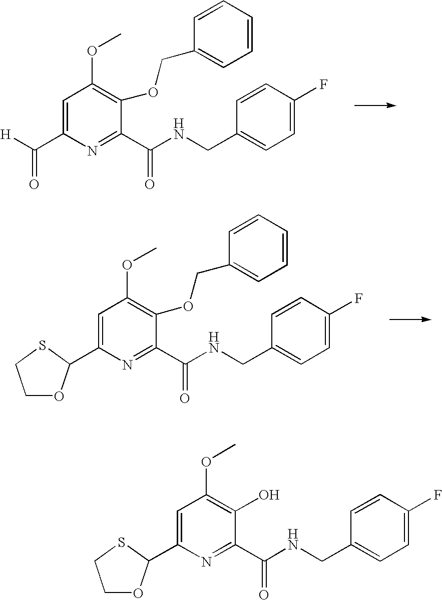

To the solution of 3-benzyloxy-6-formyl-4-methoxy-2-yl-pyridine-2-carboxylic acid 4-fluoro-benzylamide (39 mg, 0.1 mmol) in chloroform (3 mL) were added 10-camphorsulfonic acid (4.6 mg, 20 mol %) and 2-mercaptoethanol (13 uL, 0.2 mmol). The mixture was refluxed under nitrogen for 8 h. After removal of the solvent under reduced pressure, the residue was purified on silica gel column eluting with hexane and ethyl acetate (7:3) to yield a white solid, which was deprotected by TMSI to provide the title compound as an off-white solid (18 mg).

¹H NMR (400 MHz, CDCl₃): δ [ppm] 12.30 (s, 1H), 8.21 (br s, 1H), 7.28 (m, 2H), 7.08 (s, 1H), 6.99 (m, 2H), 5.93 (s, 1H), 4.53 (m, 3H), 3.92 (m, 4H), 3.18 (m, 2H).

LC/MS: m/z 365.1 (M+H⁺).

The following compounds were prepared in a similar manner:

### 3-Hydroxy-4-methoxy-6-(5-methyl-(1,3)-oxathiolan-2-yl)-pyridine-2-carboxylic acid 4-fluoro-benzylamide compound 71

This compound was obtained as a 1:2 mixture of cis:trans isomeres.

### 6-(1,3)-Dioxolan-2-yl-3-hydroxy-4-methoxy-pyridine-2-carboxylic acid 4-fluoro-benzylamide compound 72

¹H NMR (400 MHz, CDCl₃): δ [ppm] 12.45 (s, 1H), 8.35 (br s, 1H), 7.33 (m, 2H), 7.15 (s, 1H), 7.01 (m, 2H), 5.64 (s, 1H), 4.58 (d, 2H), 4.17 (m, 2H), 4.08 (m, 2H), 3.96 (s, 3H).

LC/MS: m/z 349.1 (M+H⁺).

### 6-(1,3)-Dioxan-2-yl-3-hydroxy-4-methoxy-pyridine-2-carboxylic acid 4-fluoro-benzylamide compound 76

¹H NMR (400 MHz, CDCl₃): δ [ppm] 12.41 (s, 1H), 8.37 (br s, 1H), 7.30 (m, 2H), 7.21 (s, 1H), 7.02 (m, 2H), 5.42 (s, 1H), 4.58 (d, 2H), 4.25 (m, 2H), 3.95 (m, 5H), 2.22 (m, 1H), 1.44 (m, 1H).

LC/MS: m/z 363.2 (M+H⁺).

### 3-Hydroxy-4-methoxy-6-(4-methyl-(1,3)dioxolan-2-yl)-pyridine-2-carboxylic acid 4-fluoro-benzylamide compound 73

This compound was obtained as a trans/cis mixture (1:1).

LC/MS: m/z 363.1 (M+H⁺).

### 3-Hydroxy-6-(4-hydroxymethyl-(1,3)-dioxolan-2-yl)-4-methoxy-pyridine-2-carboxylic acid 4-fluoro-benzylamide compound 75

The title compound was obtained as a trans/cis mixture.

LC/MS: m/z 379.0 (M+H⁺).

### 6-(4-Benzyloxymethyl-(1,3)-dioxolan-2-yl)-3-hydroxy-4-methoxy-pyridine-2-carboxylic acid 4-fluoro-benzylamide compound 74

The title compound was obtained as a side-product a trans/cis mixture in a ratio of about (1:1).

LC/MS: m/z 469.1 (M+H⁺).

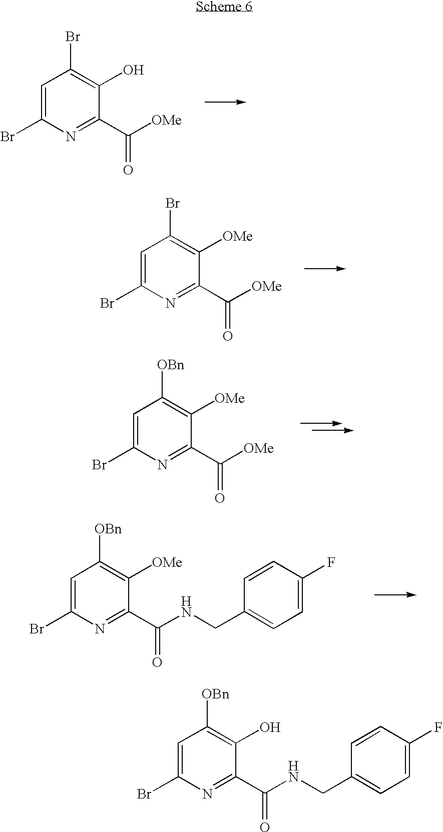

### EXAMPLE 11

### 4-Benzyloxy-6-bromo-3-hydroxy-pyridine-2-carboxylic acid 4-fluoro-benzylamide compound 25

4,6-dibromo-3-hydroxy-pyridine-2-carboxylic acid methyl ester prepared using a procedure described in Ricks, M. J. et al. WO 01/05769 A2 was methylated using the previously described alkylation procedure. The resulting compound (325 mg, 1 mmol) was further treated with 1 equivalency of sodium benzoxide to provide 4-benzyloxy-6-bromo-3-methoxy-pyridine-2-carboxylic acid methyl ester (90 mg). The monobenzylated compound was subjected to hydrolysis and amide coupling consecutively as described in example 2 and example 3 to obtain 4-benzyloxy-6-bromo-3-methoxy-pyridine-2-carboxylic acid 4-fluoro-benzylamide, which was deprotected using TMSI to yield the title compound.

¹H NMR (400 MHz, CDCl₃): δ [ppm] 12.35 (s, 1H), 8.02 (br s, 1H), 7.34 (m, 7H), 7.19 (s, 1H), 6.99 (m, 2H), 5.12 (s, 2H), 4.51 (d, 2H).

LC/MS: m/z 432.8 (M+H⁺).

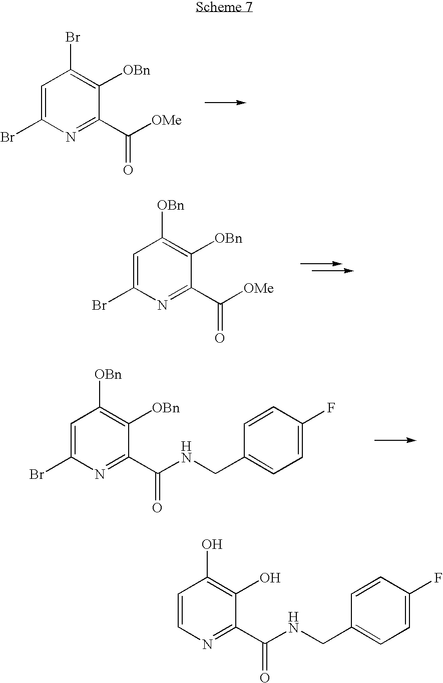

### EXAMPLE 12

### 3,4-Dihydroxy-pyridine-2-carboxylic acid 4-fluoro-benzylamide compound 11

Step I

### 3,4-Dibenzyloxy-6-bromo-pyridine-2-carboxylic acid methyl ester

Sodium hydride (62.4 mg, 1.2 mmol, 60% purity) was added to the solution of benzyl alcohol (124 ul, 1.2 mmol) in dry DMF (5 ml) at 0° C. The mixture was stirred for 10 min, and then to it was added 3-benzyloxy-4,6-dibromo-pyridine-2-carboxylic acid methyl ester (401 mg, 1 mmol). The reaction was run at rt overnight. The mixture was diluted with ether (100 ml) and washed with water (50 ml) and brine (50 ml) consecutively. The organic phase was dried with anhydrous sodium sulfate, filtered. After removal of the solvent, the residue was purified on silica gel column chromatography using hexane and ethyl acetate (85:15) to provide 125 mg of the desired product.

Step II

To a solution of 6-bromo-3,4-dibenzyloxy-pyridine-2-carboxylic acid 4-fluoro-benzylamide (35 mg) (prepared from 6-bromo-3,4-dibenzyloxy-pyridine-2-carboxylic acid by coupling as described in example 2) in 5 mL of methanol was added under nitrogen 10% palladium on charcoal (10 mg). The system was evacuated and filled with hydrogen from a balloon. The hydrogenation was taken for overnight and the mixture was filtered over celite. Solvent was removed under reduced pressure to give the desired product (12 mg, 70%) as a foam.

¹H-NMR (400 MHz, CD₃OD): δ (ppm) 8.15 (d, 1H), 7.40 (m, 2H), 7.25 (d, 1H), 7.05 (m, 2H), 4.71 (d, 2H).

### EXAMPLE 13

### 4,6-Dibromo-3-hydroxy-pyridine-2-carboxylic acid 4-fluorobenzylamide compound 18

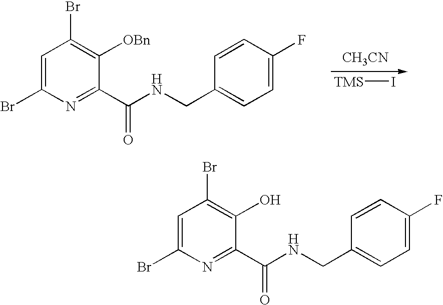

To a solution of 4,6-Dibromo-3-benzyloxy-pyridine-2-carboxylic acid 4-fluorobenzylamide obtained as described in example 12 (40 mg, 0.08 mM) in 3 mL of acetonitrile was added iodotrimethylsilane (TMSI, 60 μL, 5 eq.). The mixture was stirred at room temperature for 3 hours. Solvent was removed on evaporator and the residue was dissolved in methylene chloride. The methylene chloride solution was washed with 10% sodium thiosulfate solution, water, and brine and dried on Na₂SO₄. After removal of solvent, the residue was purified on silica gel using hexane:EtOAc 9:1 as eluant to yield 23 mg (72%) of desired product.

¹H-NMR (400 MHz, CDCl₃): δ (ppm) 12.95 (s, 1H), 8.15 (bs, 1H), 7.80 (s, 1H), 7.35 (m, 2H), 7.05 (m, 2H), 4.61 (d, 2H).

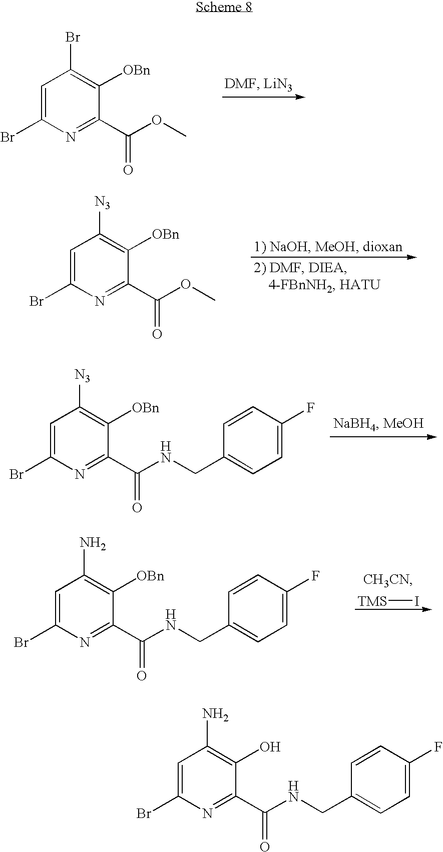

### EXAMPLE 14

### 4-Azido-3-benzyloxy-6-bromo-pyridine-2-carboxylic acid 4-fluoro-benzylamide compound 34

Step I

### 4-Azido-3-benzyloxy-6-bromo-pyridine-2-carboxylic acid methyl ester

To a solution of 4,6-Dibromo-3-benzyloxy-pyridine-2-carboxylic acid methyl ester (1 g, 2.5 mM) in 10 mL of DMF was added lithium azide (10% wet with MeOH, 164 mg, 1.3 eq.). The mixture was heated at 50° C. for overnight. Solvent was removal on evaporator and the residue was dissolved in methylene chloride. The methylene chloride solution was washed with water, brine and dried on Na₂SO₄. After removal of solvent, the residue was purified on silica gel using hexane:EtOAc 9:1 as eluant to give 440 mg (48%) of desired product and 400 mg of recovered starting material.

¹H-NMR (400 MHz, CDCl₃): δ (ppm) 7.50 (m, 2H), 7.40 (m, 3H), 7.30 (s, 1H), 5.08 (s, 2H), 3.90 (s, 3H).

Step II

### 4-Azido-3-benzyloxy-6-bromo-pyridine-2-carboxylic acid 4-fluoro-benzylamide

To a solution of 4-Azido-3-benzyloxy-6-bromo-pyridine-2-carboxylic acid methyl ester (90 mg, 0.24 mM) in 2 mL of dioxane and 1 mL of MeOH was added 0.4 mL of 10% aqueous NaOH. The mixture was stirred at RT for 2 hrs and neutralized with acetic acid. Solvent was removed on evaporator and the residue was dissolved in EtOAc. The EtOAc solution was washed with water, brine and dried on Na₂SO₄. Solvent was removed under reduced pressure to give 86 mg of free carboxylic acid, which was pure and confirmed by H-NMR. The product was used further without purification.

¹H-NMR (400 MHz, CDCl₃): δ (ppm) 7.50 (m, 2H), 7.30 (m, 3H), 7.20 (s, 1H), 5.10 (s, 2H).

The free carboxylic acid derivative (86 mg) was dissolved in 2 mL of anhydrous DMF. DIEA (0.1 mL) was added, followed by adding 4-fluorobenzylamine (57 μL, 2 eq.) and HATU (200 mg, 2 eq.). The mixture was stirred at RT for 4 hrs. Solvent was removed and residue was purified on silica gel using 5 to 20% EtOAc in hexane as eluant. It gave 80 mg (70%) of product.

¹H-NMR (400 MHz, CDCl₃): δ (ppm) 7.80 (bt, 1H), 7.45 (m, 2H), 7.30 (m, 5H), 7.10 (s, 1H), 6.96 (t, 2H), 5.10 (s, 2H), 4.53 (d, 2H).

### EXAMPLE 15

### 4-Amino-3-hydroxy-6-bromo-pyridine-2-carboxylic acid 4-fluoro-benzylamide compound 29

To a solution of 4-Azido-3-benzyloxy-6-bromo-pyridine-2-carboxylic acid 4-fluoro-benzylamide (30 mg, 0.065 mM) in 3 mL of acetonitrile was added iodotrimethylsilane (TMSI, 60 μL, 5 eq.). The mixture became dark and was stirred at room temperature for 1 hours. TLC indicated two new products were formed. Solvent was removed on evaporator and the residue was dissolved in methylene chloride. The methylene chloride solution was washed with 10% sodium thiosulfate solution, water, and brine and dried on Na₂SO₄. After removal of solvent, the residue was purified on silica gel using 5-20% EtOAc in hexane as eluant to yield 10.6 mg of the less polar product, which was identified by H-NMR and mass spectrum as 4-Amino-3-hydroxy-6-bromo-2-carboxylic acid 4-fluoro-benzylamide.

¹H-NMR (400 MHz, DMSO): δ (ppm) 12.60 (s, 1H), 9.40 (bt, 1H), 7.40 (m, 2H), 7.20 (m, 2H), 6.80 (s, 1H), 6.45 (bs, 2H), 4.45 (d, 2H).

### EXAMPLE 16

### 4-Amino-3-benzyloxy-6-bromo-pyridine-2-carboxylic acid 4-fluoro-benzylamide compound 28

4-Azido-3-benzyloxy-6-bromo-pyridine-2-carboxylic acid 4-fluoro-benzylamide (40 mg, 0.088 mM) was dissolved in 3 mL of methanol. Sodium borohydride (7 mg, 2 eq.) was added. Mixture was stirred for 20 min. and quenched with saturated NH₄Cl. The product was extracted with methylene chloride, washed with water, brine, dried with Na₂SO₄ and evaporated. The residue was purified on silica gel using hexane:EtOAc 4:1 as eluant to give 34 mg (90%) of product.

¹H-NMR (400 MHz, CDCl₃): δ (ppm) 8.05 (bt, 1H), 7.40 (m, 2H), 7.30 (m, 5H), 6.75 (s, 1H), 6.95 (t, 2H), 6.75 (s, 1H), 5.05 (s, 2H), 4.53 (d, 2H), 4.45 (bs, 2H).

### EXAMPLE 17

### 3,4,6-Trimethoxy-pyridine-2-carboxylic acid 4-fluoro-benzylamide Compound 21

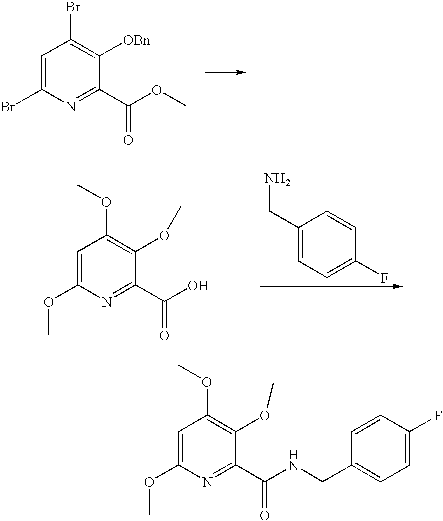 Step I

A suspension of 3-Benzyloxy-4,6-dibromo-pyridine-2-carboxylic acid methyl ester (250 mg, 0.62 mmol) in MeOH (2.5 mL) was treated with a solution of NaOMe in MeOH (2.5 mL, 10.9 mmol). The mixture was heated at 65° C. for 48 hours. The reaction was cooled to room temperature, 10% HCl (aq) was added and the mixture was evaporated to a residue that was used in the next step without further purification.

Step II

The residue obtained from the previous step was dissolved in DMF (6.2 mL) and treated with diisopropylethylamine (0.22 mL, 1.25 mmol), HATU (474 mg, 1.25 mmol) and 4-fluorobenzylamine (0.14 mL, 1.25 mmol). The solution was stirred at room temperature for 18 hours. EtOAc and water were added and the organic layer was washed with 10% HCl, 5% NaHCO₃, water and brine and dried. The solvent was then evaporated and the residue purified by silica gel column chromatography using hexanes:EtOAc as eluent to provide 3,4,6-Trimethoxy-pyridine-2-carboxylic acid 4-fluoro-benzylamide as an oil. ¹H NMR (CDCl₃, 400 MHz): 7.91 (br s, 1H), 7.32 (m, 2H), 7.05 (m, 2H), 6.35 (s, 1H), 4.60 (d, 2H), 3.88 (s, 3H), 3.87 (s, 3H), 3.86 (s, 3H).

### EXAMPLE 18

### 3-Hydroxy-4,6-dimethoxy-pyridine-2-carboxylic acid 4-fluoro-benzylamide Compound 24

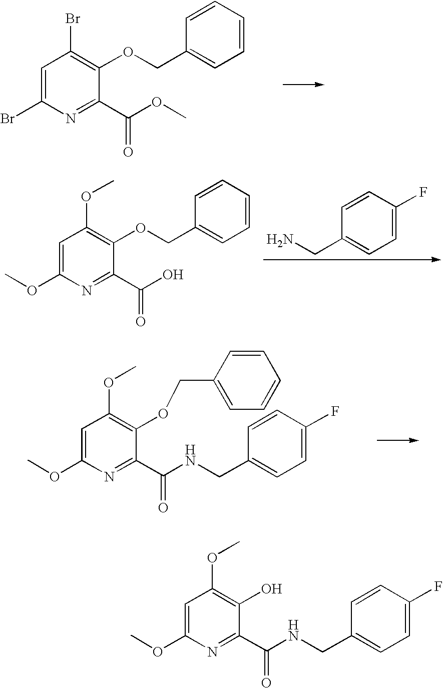 Step I

A suspension of 3-Benzyloxy-4,6-dibromo-pyridine-2-carboxylic acid methyl ester (0.250 g, 0.62 mmol) in MeOH (6.0 mL) was treated with a solution of 25% NaOMe in MeOH (0.57 mL, 2.49 mmol). The mixture was stirred for 18 hours at 60° C., cooled at room temperature and acidified with HCl. The mixture was filtered on celite and the filtrate was evaporated to a residue that was used in the next step without further purification.

Step II

The crude mixture obtained from the first step was dissolved in DMF (6.2 mL) and treated with diisopropylethylamine (0.33 mL, 1.87 mmol), HATU (0.47 mg, 1.25 mmol) and 4-fluorobenzylamine (0.14 mL, 1.25 mmol). The solution was stirred at room temperature for 18 hours. EtOAc and water were added and the organic layer was washed with 10% HCl, 5% NaHCO₃, water and brine and dried. The solvent was then evaporated and the residue purified by preparative TLC using hexanes:EtOAc as eluent to provide 3-Benzyloxy-4,6-dimethoxy-pyridine-2-carboxylic acid 4-fluoro-benzylamide as an oil. ¹H NMR (CDCl₃, 400 MHz): 7.82 (t, 1H), 7.44 (m, 2H), 7.32-7.18 (m, 5H), 6.94 (m, 2H), 6.28 (s, 1H), 4.97(s, 2H), 4.51 (d, 2H), 3.82 (m, 3H), 3.79 (s, 3H).

Step III

3-Benzyloxy-4,6-dimethoxy-pyridine-2-carboxylic acid 4-fluoro-benzylamide (13 mg, 0.0327 mmol) was dissolved in MeOH (1.0 mL) and treated with 10% Pd/C (4 mg). The mixture was stirred at room temperature under a balloon of H₂ for 18 hours. The mixture was filtered on celite and the solvent was removed to provide a residue that was purified by silica gel column chromatography using hexanes:EtOAc as eluent to furnish 3-hydroxy-4,6-dimethoxy-pyridine-2-carboxylic acid 4-fluoro-benzylamide. ¹H NMR (CDCl₃, 400 MHz): 12.0 (s, 1H), 8.03 (br s, 1H), 7.33 (m, 2H), 7.04 (m, 2H), 6.34 (s, 1H), 4.60 (d, 2H), 3.91(s, 3H), p. 3.83 (s, 3H).

### EXAMPLE 19

### 5′-Hydroxy-4′-methoxy-3,4,5,6-tetrahydro-2H-[1,2′]bipyridinyl-6′-carboxylic acid 4-fluoro-benzylamide compound 32

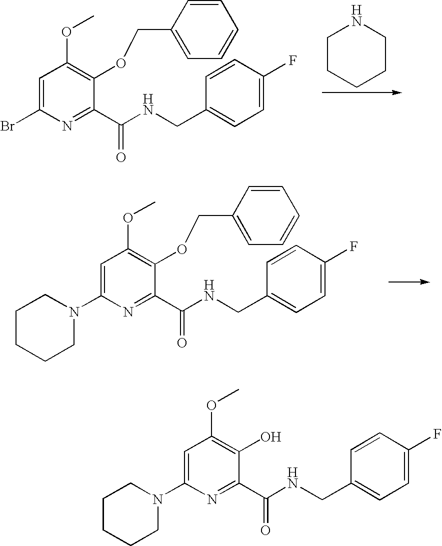 Step I

A suspension of 3-benzyloxy-6-bromo-4-methoxy-pyridine-2-carboxylic acid 4-fluoro-benzylamide (49.6 mg, 0.11 mmol), Cs₂CO₃ (50.8 mg, 0.156 mmol) and rac-BINAP (6.9 mg, 0.011 mmol) in dioxane (2.2 mL) was treated with Pd(OAc)₂ (1.2 mg, 0.006 mmol) and piperidine (13.2 μL, 0.134 mmol). The reaction was stirred at 110° C. for 18 hours, cooled at room temperature and the mixture was filtered on a pad of silica gel. The solution was evaporated to a residue that was purified by preparative TLC using hexanes and EtOAc as eluent to provide 5′-benzyloxy-4′-methoxy-3,4,5,6-tetrahydro-2H-[1,2′]bipyridinyl-6′-carboxylic acid 4-fluoro-benzylamide. ¹H NMR (CDCl₃, 400 MHz): 7.94 (t, 1H), 7.54 (m, 2H), 7.37-7.28 (m, 5H), 6.99 (m, 2H), 6.28 (s, 1H), 5.01 (s, 2H), 4.58 (d, 2H), 3.86 (s, 3H), 3.46 (m, 4H), 1.64 (m, 6H).

Step II

5′-Benzyloxy-4′-methoxy-3,4,5,6-tetrahydro-2H-[1,2′]bipyridinyl-6′-carboxylic acid 4-fluoro-benzylamide (5.9 mg, 0.013 mmol) was dissolved in MeOH (1.0 mL) and treated with 10% Pd/C (2 mg). The mixture was stirred at room temperature under a balloon of H₂ for 18 hours. The mixture was filtered over celite and the solvent was removed to provide a residue that was purified by silica gel column chromatography using hexanes:EtOAc as eluent to furnish 5-hydroxy-4′-methoxy-3,4,5,6-tetrahydro-2H-[1,2′]bipyridinyl-6′-carboxylic acid 4-fluoro-benzylamide. ¹H NMR (CDCl₃, 400 MHz): 11.6 (s, 1H), 8.09 (br s, 1H), 7.25 (m, 2H), 6.96 (m, 2H), 6.31 (s, 1H), 4.52 (d, 2H), 3.84(s, 3H), 3.28 (m, 4H), 1.57 (m, 6H).

### 3-Hydroxy-4-methoxy-6-(tetrahydro-furan-2-yl)-pyridine-2-carboxylic acid 4-methyl-benzylamide compound 50

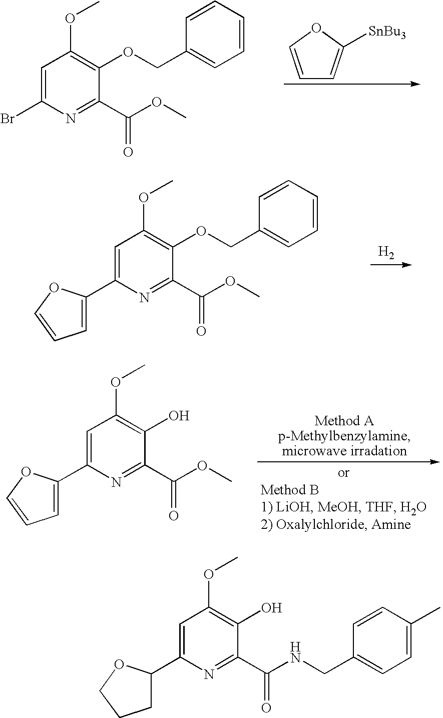 Step I

To a stirring solution of 3-benzyloxy-6-bromo-4-methoxy- pyridine-2-carboxylic acid methyl ester (1.75 g, 4.97 mmol) in dry tetrahydrofuran (50.0 mL) was added tetrakis(triphenylphosphine)palladium (0) (346 mg, 0.30 mmol) and 2-(tributylstannyl)furan (3.13 mL, 9.94 mmol). The mixture was stirred for 20 hours at 70° C., cooled at room temperature and concentrated to dryness. The residue was purified by flash chromatography eluting first with 10% methylene chloride/hexanes, then 10% to 20% ethyl acetate/hexanes to afford 3-benzyloxy-6-furan-2-yl-4-methoxy-pyridine-2-carboxylic acid methyl ester (1.48 g, 88%) as a yellow solid.

¹H NMR (400 MHz, CDCl₃): δ [ppm] 7.48 (m, 3H), 7.35 (m, 4H), 7.06 (m, 1H), 6.51 (m, 1H), 5.10 (s, 2H), 4.01 (s, 3H), 3.90 (s, 3H).

Step II

To a stirring solution of 3-benzyloxy-6-furan-2-yl-4-methoxy-pyridine-2-carboxylic acid methyl ester (1.66 g, 4.90 mmol) in methanol (25 mL) and ethyl acetate (25 mL) was added acetic acid (0.1 mL) and 10% Pd/C (400 mg). The resulting mixture was stirred 20 hours under a balloon of H₂ but only the benzyl was removed. The mixture was filtered through celite and concentrated. The residue obtained was treated a second time under identical conditions, stirring 2 days under a balloon of H₂. The mixture was filtered through celite and concentrated. The residue obtained was purified by flash chromatography eluting with 2% methanol/methylene chloride to afford 3-hydroxy-4-methoxy-6-(tetrahydro-furan-2-yl)-pyridine-2-carboxylic acid methyl ester (0.94 g, 75%) as a white solid.

¹H NMR (400 MHz, CDCl₃): δ [ppm] 10.85 (s, 1H), 7.16 (s, 1H), 4.96 (m, 1H), 4.07 (m, 1H), 4.02 (s, 3H), 3.96 (s, 3H), 3.93 (m, 1H), 2.42 (m, 1H), 1.97 (m, 3H).

Step III

Method A

A solution of 3-hydroxy-4-methoxy-6-(tetrahydro-furan-2-yl)-pyridine-2-carboxylic acid methyl ester (54 mg, 0.214 mmol) and 4-methyl-benzylamine (0.14 mL, 1.07 mmol) in acetonitrile (1 mL) was heated 15 minutes at 200° C. under microwave irradiation. The mixture was concentrated to dryness and the residue was purified by flash chromatography eluting with 0% to 0.5% methanol/methylene chloride to yield 3-Hydroxy-4-methoxy-6-(tetrahydro-furan-2-yl)-pyridine-2-carboxylic acid 4-methyl-benzylamide compound 50 as a colorless oil (54 mg, 74%).

¹H NMR (400 MHz, CDCl₃): δ [ppm] 12.29 (s, 1H), 8.30 (br s, 1H), 7.25 (d, 2H), 7.15 (d, 2H), 7.05 (S, 1H), 4.82 (m, 1H), 4.58 (m, 2H), 4.05 (m, 1H), 3.94 (s, 3H), 3.92 (m, 1H), 2.34 (s, 3H), 2.29 (m, 1H), 1.94 (m, 3H).

The following compounds were prepared in the same manner using method A:

### 3-Hydroxy-4-methoxy-6-(tetrahydro-furan-2-yl)-pyridine-2-carboxylic acid 4-methoxy-benzylamide compound 51

¹H NMR (400 MHz, CDCl₃): δ [ppm] 12.29 (s, 1H), 8.27 (br s, 1H), 7.27 (d, 2H), 7.04 (s, 1H), 6.87 (d, 2H), 4.81 (m, 1H), 4.54 (m, 2H), 4.05 (m, 1H), 3.94 (s, 3H), 3.92 (m, 1H), 3.78 (s, 3H), 2.28 (m, 1H), 1.93 (m, 3H).

### 3-Hydroxy-4-methoxy-6-(tetrahydro-furan-2-yl)-pyridine-2-carboxylic acid 4-trifluoromethoxy-benzylamide compound 52

¹H NMR (400 MHz, CDCl₃): δ [ppm] 12.15 (s, 1H), 8.39 (br s, 1H), 7.37 (d, 2H), 7.19 (d, 2H), 7.06 (s, 1H), 4.83 (m, 1H), 4.62 (m, 2H), 4.06 (m, 1H), 3.95 (s, 3H), 3.92 (m, 1H), 2.32 (m, 1H), 1.94 (m, 3H).

### 3-Hydroxy-4-methoxy-6-(tetrahydro-furan-2-yl)-pyridine-2-carboxylic acid 4-trifluoromethyl-benzylamide compound 53

¹H NMR (400 MHz, CDCl₃): δ [ppm] 12.09 (s, 1H), 8.43 (br s, 1H), 7.59 (d, 2H), 7.45 (d, 2H), 7.06 (s, 1H), 4.83 (m, 1H), 4.68 (m, 2H), 4.06 (m, 1H), 3.95 (s, 3H), 3.92 (m, 1H), 2.31 (m, 1H), 1.95 (m, 3H).

### 3-Hydroxy-4-methoxy-6-(tetrahydro-furan-2-yl)-pyridine-2-carboxylic acid 2-fluoro-benzylamide compound 54

¹H NMR (400 MHz, CDCl₃): δ [ppm] 12.17 (s, 1H), 8.38 (br s, 1H), 7.38 (t, 1H), 7.26 (m, 1H), 7.09 (m, 2H), 7.06 (s, 1H), 4.84 (m, 1H), 4.67 (m, 2H), 4.04 (m, 1H), 3.93 (s, 3H), 3.92 (m, 1H), 2.32 (m, 1H), 1.95 (m, 3H).

### 3-Hydroxy-4-methoxy-6-(tetrahydro-furan-2-yl)-pyridine-2-carboxylic acid 3-fluoro-benzylamide compound 55

¹H NMR (400 MHz, CDCl₃): δ [ppm] 12.14 (s, 1H), 8.37 (br s, 1H), 7.30 (m, 1H), 7.11 (m, 1H), 7.06 (s, 1H), 7.03 (d, 1H), 6.96 (m, 1H), 4.83 (m, 1H), 4.62 (m, 2H), 4.05 (m, 1H), 3.94 (s, 3H), 3.92 (m, 1H), 2.30 (m, 1H), 1.95 (m, 3H).

### 3-Hydroxy-4-methoxy-6-(tetrahydro-furan-2-yl)-pyridine-2-carboxylic acid 2,4-difluoro-benzylamide compound 56

¹H NMR (400 MHz, CDCl₃): δ [ppm] 12.10 (s, 1H), 8.36 (br s, 1H), 7.36 (m, 1H), 7.05 (s, 1H), 6.85 (m, 2H), 4.84 (m, 1H), 4.62 (m, 2H), 4.04 (m, 1H), 3.94 (s, 3H), 3.92 (m, 1H), 2.32 (m, 1H), 1.95 (m, 3H).

### 3-Hydroxy-4-methoxy-6-(tetrahydro-furan-2-yl)-pyridine-2-carboxylic acid 3,4-difluoro-benzylamide compound 57

¹H NMR (400 MHz, CDCl₃): δ [ppm] 12.09 (s, 1H), 8.39 (br s, 1H), 7.13 (m, 3H), 7.06 (s, 1H), 4.84 (m, 1H), 4.57 (m, 2H), 4.05 (m, 1H), 3.95 (s, 3H), 3.92 (m, 1H), 2.32 (m, 1H), 1.95 (m, 3H).

### 3-hydroxy-4-methoxy-6-(tetrahydro-furan-2-yl)-pyridine-2-carboxylic acid (4-fluoro-benzyl)-methyl-amide compound 58

¹H NMR (400 MHz, CDCl₃): δ [ppm] (presence of two rotomers 1:2 ratio) 12.25 (s, 0.66H), 12.02 (s, 0.33H), 7.30 (m, 2H), 7.01 (m, 3H), 5.31 (dd, 1.33H), 4.86 (m, 0.33H), 4.71 (m, 0.66H), 4.65 (m, 0.66H), 4.02 (m, 0.33H), 3.94 (s, 3H), 3.88 (m, 1.66H), 3.47 (s, 1.33H), 3.01 (s, 1.66H), 2.29 (m, 0.33H), 1.96 (m, 1.66H), 1.75 (m, 2H).

### 3-Hydroxy-4-methoxy-6-(tetrahydro-furan-2-yl)-pyridine-2-carboxylic acid [1-(4-fluoro-phenyl)-ethyl]-amide compound 59

¹H NMR (400 MHz, CDCl₃): δ [ppm] 12.17 (s, 1H), 8.20 (d, 1H), 7.34 (m, 2H), 7.04 (s, 1H), 7.02 (m, 2H), 5.20 (m, 1H), 4.84 (m, 1H), 4.05 (m, 1H), 3.95 (m, 1H), 3.93 (s, 3H), 3.78 (s, 3H), 2.33 (m, 1H), 1.96 (m, 3H), 1.59 (d, 3H).

### 3-Hydroxy-4-methoxy-6-(tetrahydro-furan-2-yl)-pyridine-2-carboxylic acid 4-bromo-benzylamide compound 60

¹H NMR (400 MHz, CDCl₃): δ [ppm] 12.14 (s, 1H), 8.33 (br s, 1H), 7.37 (d, 2H), 7.22 (d, 2H), 7.06 (s, 1H), 4.82 (m, 1H), 4.57 (m, 2H), 4.06 (m, 1H), 3.95 (s, 3H), 3.92 (m, 1H), 2.31 (m, 1H), 1.95 (m, 3H).

### 3-Hydroxy-4-methoxy-6-(tetrahydro-furan-2-yl)-pyridine-2-carboxylic acid 4-chloro-benzylamide compound 61

¹H NMR (400 MHz, CDCl₃): δ [ppm] 12.16 (s, 1H), 8.36 (br s, 1H), 7.31 (m, 4H), 7.06 (s, 1H), 4.83 (m, 1H), 4.59 (m, 2H), 4.04 (m, 1H), 3.95 (s, 3H), 3.92 (m, 1H), 2.31 (m, 1H), 1.95 (m, 3H).

Method B:

### 3-Hydroxy-4-methoxy-6-(tetrahydro-furan-2-yl)-pyridine-2-carboxylic acid (pyridin-2-ylmethyl)-amide 40

A solution of 3-hydroxy-4-methoxy-6-(tetrahydro-furan-2-yl)-pyridine-2-carboxylic acid methyl ester (295 mg, 1.16 mmol) in 3:2:1 solution of THF:MeOH:H₂O (12 mL) was treated with lithium hydroxide (98 mg, 2.33 mmol). The mixture was stirred at 50° C. for 3 hours and concentrated. The residue was dissolved in water, acidified to pH 3-4 with HCl and the product was extracted with CHCl₃. The combined organic layers were washed with brine, dried over Na₂SO₄ and evaporated to a white solid (248.4 mg, 89%) that was used in the next step without further purification. ¹H NMR (400 MHz, CD₃OD): δ [ppm] 7.06 (s, 1H), 5.15 (m, 1H), 4.13 (q, 1H), 4.06 (s, 3H), 3.98 (q, 1H), 2.57 (m, 1H), 2.07 (m, 1H), 1.97 (m, 1H), 1.82 (m, 1H).

A solution of 3-hydroxy-4-methoxy-6-(tetrahydro-furan-2-yl)-pyridine-2-carboxylic acid (95.6 mg, 0.4 mmol) in CH₂Cl₂ (4 mL) was treated with 2M solution of oxalyl chloride in CH₂Cl₂ (0.63 mL, 1.25 mmol) and 2 drops of DMF. The reaction was stirred at room temperature for 3 hours. The solvent was evaporated and the residue was left to dry on the pump. The acid chloride formed (0.1 mmol) was dissolved in DMF (1 mL) and treated with 2-aminomethylpyridine (21 μL, 0.2 mmol) and Et₃N (28 μL, 0.2 mmol). The mixture was stirred at room temperature for 18 hours. The solvent was evaporated to a residue which was dissolved in EtOAc, washed with 5% NaHCO₃, water and brine and dried over Na₂SO₄. After removal of the solvent, the residue was purified by preparative TLC using hexane and ethyl acetate to provide 3-hydroxy-4-methoxy-6-(tetrahydro-furan-2-yl)-pyridine-2-carboxylic acid (pyridin-2-ylmethyl)-amide (10.8 mg, 33%), compound 40 as a yellow solid.

¹H NMR (400 MHz, CDCl₃): δ [ppm] 12.14 (s, 1H), 8.84 (br s, 1H), 8.53 (m, 1H), 7.63 (m, 1H), 7.29 (d, 1H), 7.16 (dd, 1H), 7.00 (s, 1H), 4.81 (m, 1H), 4.69 (m, 2H), 4.01 (m, 1H), 3.90 (m, 1H), 3.89 (s, 3H), 2.28 (m, 1H), 1.95 (m, 3H).

The following compounds were prepared in a similar manner using method B:

### 3-Hydroxy-4-methoxy-6-(tetrahydro-furan-2-yl)-pyridine-2-carboxylic acid benzylamide compound 38

¹H NMR (400 MHz, CDCl₃): δ [ppm] 12.2 (s, 1H), 8.28 (br s, 1H), 7.31-7.19 (m, 5H), 7.00 (s, 1H), 4.77 (m, 1H), 4.57 (m, 2H), 3.98 (m, 1H), 3.89 (s, 3H), 3.87 (m, 1H), 2.25 (m, 1H), 1.90 (m, 3H).

### 3-Hydroxy-4-methoxy-6-(tetrahydro-furan-2-yl)-pyridine-2-carboxylic acid [2-(4-fluoro-phenyl)-ethyl]-amide compound 39

¹H NMR (400 MHz, CDCl₃): δ [ppm] 12.18 (s, 1H), 8.00 (br s, 1H), 7.14 (m, 2H), 6.98 (s, 1H), 6.94 (m, 2H), 4.75 (m, 1H), 3.95 (m, 1H), 3.90 (m, 1H), 3.88 (s, 3H), 3.60 (q, 2H), 2.84 (t, 2H), 2.21 (m, 1H), 1.88 (m, 3H).

### 3-Hydroxy-4-methoxy-6-(tetrahydro-furan-2-yl)-pyridine-2-carboxylic acid cyclohexylmethyl-amide compound 41

¹H NMR (400 MHz, CDCl₃): δ [ppm] 12.35 (s, 1H), 8.03 (br s, 1H), 6.98 (s, 1H), 4.81 (t, 1H), 4.00 (m, 1H), 3.90 (m, 1H), 3.88 (s, 3H), 3.20 (m, 2H), 2.28 (m, 1H), 1.92 (m, 3H), 1.67-1.53 (m, 6H), 1.21-1.08 (m, 3H), 0.95 (m, 2H).

### 3-Hydroxy-4-methoxy-6-(tetrahydro-furan-2-yl)-pyridine-2-carboxylic acid 4-nitro-benzylamide compound 80

¹H NMR (400 MHz, CDCl₃): δ [ppm] 11.96 (s, 1H), 8.48 (br. s, 1H), 8.20 (d, 2H), 7.51 (d, 2H), 7.07 (s, 1H), 4.83 (m, 1H), 4.72 (m, 2H), 4.05 (m, 1H), 3.95 (s, 3H), 3.93 (m, 1H), 2.33 (m, 1H), 1.96 (m, 3H).

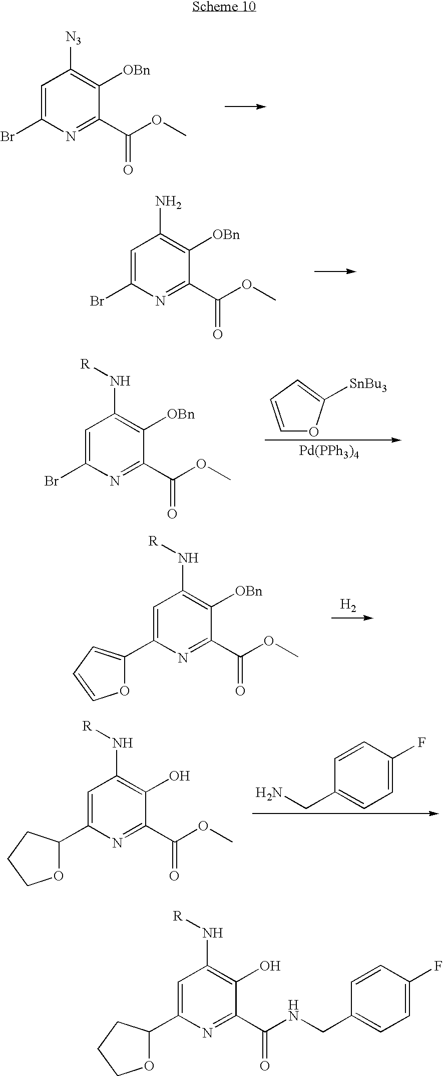

### 4-Acetylamino-3-hydroxy-6(tetrahydro-furan-2-yl)-pyridine-2-carboxylic acid 4-fluoro-benzylamide compound 44

Step I

A suspension of 4-azido-3-benzyloxy-6-bromo-pyridine-2-carboxylic acid methyl ester (1.25 g, 3.43 mmol) in MeOH (34 mL) at 0° C. was treated with NaBH₄ (0.39 g, 10.3 mmol). The mixture was stirred at room temperature for 18 hours. EtOAc and NH₄CL (aq) were added and the product was extracted with EtOAc. The combined organic layers were washed with brine, dried over Na₂SO₄ and the solvent was evaporated to provide 4-amino-3-benzyloxy-6-bromo-pyridine-2-carboxylic acid methyl ester (0.968 g, 84%) as a white solid.

¹H NMR (400 MHz, CDCl₃): δ [ppm] 7.44-7.36 (m, 5H), 6.86 (s, 1H), 4.99 (s, 2H), 4.49 (br s, 2H), 3.99 (s, 3H).

Step II

A solution of 4-amino-3-benzyloxy-6-bromo-pyridine-2-carboxylic acid methyl ester (69.9 mg, 0.2 mmol) in acetic anhydride (0.5 mL) was stirred at 100° C. for 18 hours. After removal of the solvent, the residue was purified on silica gel column chromatography using hexane and ethyl acetate as eluent to provide 4-acetylamino-3-benzyloxy-6-bromo-pyridine-2-carboxylic acid methyl ester (59.3 mg, 75%) as a colorless oil.

¹H NMR (400 MHz, CDCl₃): δ [ppm] 8.60 (s, 1H), 8.59 (br s, 1H), 7.44-7.25 (m, 5H), 5.08 (s, 2H), 3.99 (s, 3H), 1.83 (s, 3H).

Step III

To a solution of 4-acetylamino-3-benzyloxy-6-bromo-pyridine-2-carboxylic acid methyl ester (59 mg, 0.16 mmol) in THF (1.6 mL) were added 2-(tributylstannyl)furan (98 μL, 0.31 mmol), and Pd(PPh₃)₄ (18 mg, 0.01 mmol). Under nitrogen, the mixture was stirred at 70° C. for 18 hours. After removal of the solvent, the residue was purified on silica gel column chromatography using hexane and ethyl acetate to provide 4-acetylamino-3-benzyloxy-6-furan-2-yl-pyridine-2-carboxylic acid methyl ester (41.9 mg, 74%) as a colorless oil that solidified upon standing.

¹H NMR (400 MHz, CDCl₃): δ [ppm] 8.77 (s, 1H), 7.61 (br s, 1H), 7.51 (m, 1H), 7.41 (m, 5H), 7.03 (m, 1H), 6.49 (m, 1H), 5.08 (s, 2H), 4.02 (s, 3H), 1.86 (s, 3H).

Step IV

A solution of 4-acetylamino-3-benzyloxy-6-furan-2-yl-pyridine-2-carboxylic acid methyl ester (41.9 mg, 0.11 mmol) in a mixture of 1:1 MeOH:EtOAc (1.2 mL) was treated with acetic acid (5 drops) and 10% Pd/C (13 mg). The mixture was stirred at room temperature under a balloon of H₂ for 18 hours. The mixture was filtered on celite and the solvent was removed to provide 4-acetylamino-3-hydroxy-6-(tetrahydro-furan-2-yl)-pyridine-2-carboxylic acid methyl ester (32 mg, 99%) that was used in the next step without further purification.

¹H NMR (400 MHz, CDCl₃): δ [ppm] 8.55 (s, 1H), 7.97 (br s, 1H), 4.92 (br s, 1H), 4.07 (m, 1H), 3.98 (s, 3H), 3.88 (m, 1H), 2.33 (m, 1H), 2.20 (s, 3H), 1.89 (m, 3H).

Step V

A solution of 4-acetylamino-3-hydroxy-6-(tetrahydro-furan-2-yl)-pyridine-2-carboxylic acid methyl ester (32 mg, 0.11 mmol) in toluene (1.0 mL) was treated with 4-fluorobenzylamine (65 □L, 0.57 mmol). The heterogeneous mixture was heated in microwave at 170° C. for 10 min. The solvent was than evaporated and the residue was purified on silica gel column chromatography using CH₂Cl₂ and MeOH as eluent and repurified by preparative TLC using CH₂Cl₂ and MeOH as eluent to provide 4-Acetylamino-3-hydroxy-6(tetrahydro-furan-2-yl)-pyridine-2-carboxylic acid 4-fluoro-benzylamide (27 mg, 63%) as a white solid.

¹H NMR (400 MHz, DMSO-d₆): (2 confomers were observed) δ [ppm] 13.05 (br s, 0.2H), 12.20 (m, 0.8H), 9.62 (m, 1H), 8.40 (s, 0.2H), 8.18 (s, 0.8H), 7.38 (m, 2H), 7.18 (m, 2H), 4.85 (m, 1H), 4.60 (m, 2H), 3.92 (m, 0.2H), 3.80 (m, 0.2H), 3.40 (m, 1.6H), 2.35 (m, 1H), 2.20 (s, 2.4H), 2.15 (s, 0.6H), 2.00-1.40 (m, 3H).

The following compounds were prepared in a similar manner:

### 3-Hydroxy-4-phenylacetylamino-6(tetrahydro-furan-2-yl)-pyridine-2-carboxylic acid 4-fluoro-benzylamide compound 48

¹H NMR (400 MHz, CDCl₃): (2 confomers were observed) δ [ppm] 9.22 (br s, 1H), 8.21 (br s, 1H), 7.40-7.20 (m, 5H), 7.14 (m, 2H), 6.96 (m, 3H), 5.65 (br s, 1H), 4.85-4.50 (m, 2H), 4.38 (d, 2H), 3.80 (s, 1H), 3.60 (s, 1H), 3.50-3.22 (m, 1H), 2.40 (m, 1H), 2.05-1.60 (m, 3H).

### 6-Furan-2-yl-3-hydroxy-4-phenylmethanesulfonylamino-pyridine-2-carboxylic acid 4-fluoro-benzylamide compound 49

¹H NMR (400 MHz, CDCl₃): δ [ppm] 12.58 (s, 1H), 7.68 (s, 1H), 7.43 (m, 1H), 7.33-7.19 (m, 6H), 7.00 (m, 3H), 6.77 (d, 1H), 6.42 (dd, 1H), 4.57 (d, 2H), 4.40 (s, 2H).

### 3′-Hydroxy-6′-(tetrahydro-furan-2-yl)-3,4,5,6-tetrahydro-2H-(1,4′)bipyridinyl-2′carboxylic acid 4-fluoro-benzylamide compound 81

¹H NMR (400 MHz, CDCl₃): δ [ppm] 12.63 (s, 1H), 8.50 (t, 1H), 7.33 (m, 2H), 7.00 (m, 2H), 6.90 (s, 1H), 4.80 (t, 1H), 4.60 (m, 2H), 4.04 (m, 1H), 3.90 (m, 1H), 3.20 (m 4H), 2.30 (m, 1H), 1.95 (m, 4H), 1.75 (m, 5H), 1.60 (m, 2H).

### 6-Furan-2-yl-3-hydroxy-4-methylsulfanyl-pyridine-2-carboxylic acid 4-fluoro-benzylamide compound 46

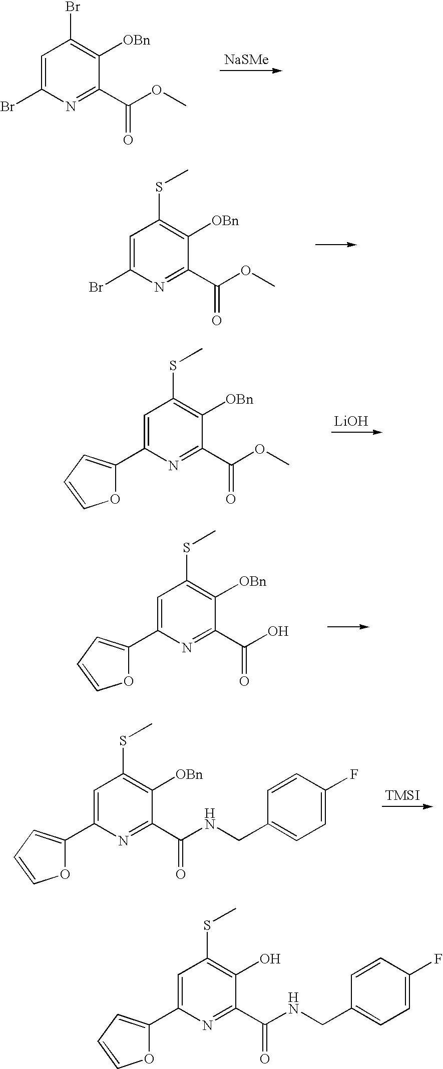 Step I

A solution of 3-benzyloxy-4,6-dibromo-pyridine-2-carboxylic acid methyl ester (451 mg, 1.12 mmol) in DMF (11 mL) was treated with sodium thiomethoxide (87 mg, 12 mmol). The mixture was stirred at 60° C. for 18 hours. After removal of the solvent, water and EtOAc were added and the product was extracted with EtOAc. The organic layers were combined, washed with brine, dried over Na₂SO₄ and the solvent was evaporated to provide 3-benzyloxy-6-bromo-4-methylsulfanyl-pyridine-2-carboxylic acid methyl ester (414 mg, 99%) as a pale yellow solid.

¹H NMR (400 MHz, CDCl₃): δ [ppm] 7.50 (m, 2H), 7.40-7.30 (m, 4H), 5.08 (s, 2H), 3.91 (s, 3H), 2.46 (s, 3H).

Step II

To a solution of 3-benzyloxy-6-bromo-4-methylsulfanyl-pyridine-2-carboxylic acid methyl ester (414 mg, 1.12 mmol) in THF (11 mL) were added 2-(tributylstannyl)furan (0.7 mL, 2.25 mmol), and Pd(PPh₃)₄ (130 mg, 0.11 mmol). Under nitrogen, the mixture was stirred at 70° C. for 18 hours. After removal of the solvent, the residue was purified on silica gel column chromatography using hexane and ethyl acetate to provide 3-benzyloxy-6-furan-2-yl-4-methylsulfanyl-pyridine-2-carboxylic acid methyl ester (346 mg, 87%) as a pale yellow solid.

¹H NMR (400 MHz, CDCl₃): δ [ppm] 7.55-7.51 (m, 4H), 7.41-7.35 (m, 3H), 7.08 (d, 1H), 6.52 (dd, 1H), 5.10 (s, 2H), 3.93 (s, 3H), 2.54 (s, 3H).

Step III

A solution of 3-benzyloxy-6-furan-2-yl-4-methylsulfanyl-pyridine-2-carboxylic acid methyl ester (201.6 mg, 0.57 mmol) in 4:1 solution of Dioxane: H₂O (6 mL) was treated with lithium hydroxide (71.5 mg, 1.7 mmol). The mixture was stirred at 50° C. for 2 hours. After removal of the solvents, the residue was dissolved in water, acidified to pH 3-4 with HCl and the product was extracted with EtOAc. The combined organic layers were washed with brine, dried over Na₂SO₄ and evaporated to furnish 3-benzyloxy-6-furan-2-yl-4-methylsulfanyl-pyridine-2-carboxylic acid (190 mg, 98%) that was used in the next step without further purification.

¹H NMR (400 MHz, CDCl₃): δ [ppm] 7.55 (m, 3H), 7.35 (m, 4H), 7.05 (d, 1H), 6.50 (dd, 1H), 5.10 (s, 2H), 2.48 (s, 3H).

Step IV

3-Benzyloxy-6-furan-2-yl-4-methylsulfanyl-pyridine-2-carboxylic acid was dissolved in DMF (5.6 mL) and treated with diisopropylethylamine (0.29 mL, 1.67 mmol), HBTU (316 mg, 0.83 mmol) and 4-fluorobenzylamine (95 □L, 0.83 mmol). The solution was stirred at room temperature for 18 hours. EtOAc and water were added and the organic layer was washed with 10% HCl, 5% NaHCO₃, water and brine and dried over Na₂SO₄. The solvent was then evaporated and the residue purified by silica gel column chromatography using hexanes:EtOAc as eluent to provide 3-benzyloxy-6-furan-2-yl-4-methylsulfanyl-pyridine-2-carboxylic acid 4-fluoro-benzylamide (176 mg, 70%) as a pale yellow solid.

¹H NMR (400 MHz, CDCl₃): δ [ppm] 8.07 (br s, 1H), 7.55-7.45 (m, 3H), 7.34-7.21 (m, 4H), 7.19 (m, 2H), 6.94 (m, 3H), 6.50 (dd, 1H), 5.12 (s, 2H), 4.57 (d, 2H), 2.44 (s, 3H).

Step V

A suspension of 3-benzyloxy-6-furan-2-yl-4-methylsulfanyl-pyridine-2-carboxylic acid 4-fluoro-benzylamide (20 mg, 0.04 mmol) in CH₃CN (1.0 mL) was treated with TMSI (19 □L, 0.13 mmol). After stirring at room temperature for 2 hours, the solvent was removed and the residue was dissolved in 1N HCl and extracted with EtOAc. The combined organic layers were washed with brine, dried over Na₂SO₄ and evaporated to a residue that was purified by silica gel column chromatography using hexanes:EtOAc as eluent to provide 6-furan-2-yl-3-hydroxy-4-methylsulfanyl-pyridine-2-carboxylic acid 4-fluoro-benzylamide (10.3 mg, 64%) as a brown oil that solidified upon standing.

¹H NMR (400 MHz, CDCl₃): δ [ppm] 12.72 (s, 1H), 8.37 (br s, 1H), 7.52 (s; 1H), 7.47 (s, 1H), 7.31 (m, 2H), 7.04 (m, 2H), 6.86 (m, 1H), 6.48 (m, 1H), 4.61 (d, 2H), 2.53 (s, 3H).

### 3-Hydroxy-4-methanesulfonyl-6-(tetrahydro-furan-2-yl)-pyridine-2-carboxylic acid 4-fluoro-benzylamide compound 45

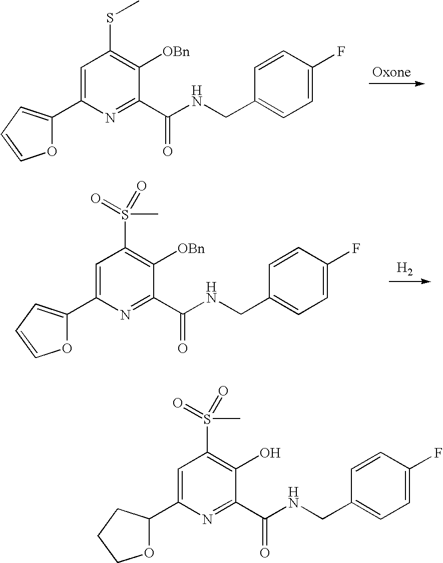 Step I

A solution of 3-benzyloxy-6-furan-2-yl-4-methylsulfanyl-pyridine-2-carboxylic acid 4-fluoro-benzylamide (52.5 mg, 0.12 mmol) in THF (0.6 mL) was treated with a solution of Oxone (215 mg, 0.35 mmol) in water (0.6 mL). The slurry mixture was stirred at room temperature for 18 hours. EtOAc and water were added and the organic layer was washed with NaOH (0.5N) and brine, dried over Na₂SO₄ and evaporated to a residue that was purified by preparative TLC using hexanes:EtOAc as eluent to provide 3-benzyloxy-6-furan-2-yl-4-methanesulfonyl-pyridine-2-carboxylic acid 4-fluoro-benzylamide (17.8 mg, 32%) as a white solid.

¹H NMR (400 MHz, CDCl₃): δ [ppm] 8.37 (s, 1H), 8.21 (br s, 1H), 7.74 (m, 2H), 7.56 (m, 1H), 7.44-7.35 (m, 5H), 7.04 (m, 3H), 6.54 (m, 1H), 5.35 (s, 2H), 4.68 (d, 2H), 3.24 (s, 3H).

Step II

3-Benzyloxy-6-furan-2-yl-4-methanesulfonyl-pyridine-2-carboxylic acid 4-fluoro-benzylamide was treated in the hydrogenation conditions described above to yield 3-hydroxy-4-methanesulfonyl-6-(tetrahydro-furan-2-yl)-pyridine-2-carboxylic acid 4-fluoro-benzylamide (5.0 mg, 34%) as a colorless oil.

¹H NMR (400 MHz, CDCl₃): δ [ppm] 13.40 (s, 1H), 8.21 (br s, 1H), 8.05 (s, 1H), 7.32 (m, 2H), 7.00 (m, 2H), 4.88 (m, 1H), 4.58 (m, 2H), 4.05 (m, 1H), 3.88 (m, 1H), 3.22 (s, 3H), 2.30 (m, 1H), 1.90 (m, 3H).

### 3-Hydroxy-6-methoxy-4-vinyl-pyridine-2-carboxylic acid 4-fluoro-benzylamide compound 47

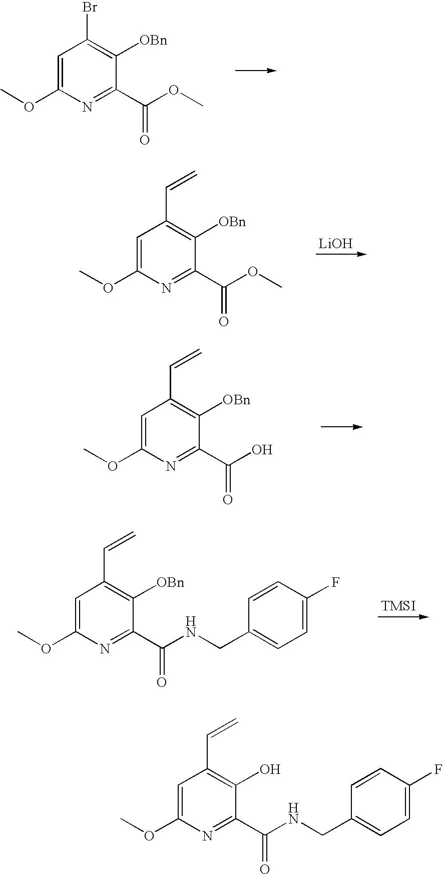 Step I

A solution of 3-benzyloxy-4-bromo-6-methoxy-pyridine-2-carboxylic acid methyl ester (177 mg, 0.50 mmol) in THF (5.0 mL) was treated with tributyl(vinyl)tin (0.29 mL, 1.0 mmol) and dichlorobis(triphenylphosphine)palladium (35 mg, 0.05 mmol). The mixture was stirred at 70° C. for 18 hours. The solvent was removed and the residue was purified by silica gel column chromatography using hexanes:EtOAc as eluent to provide 3-benzyloxy-6-methoxy-4-vinyl-pyridine-2-carboxylic acid methyl ester (43.5 mg, 30%) as an oil.

¹H NMR (400 MHz, CDCl₃): δ [ppm] 7.45-7.34 (m, 5H), 6.95 (s, 1H), 6.91 (m, 1H), 5.91 (m, 1H), 5.50 (m, 1H), 4.91 (s, 2H), 3.93 (s, 3H), 3.90 (s, 3H).

Step II

3-Benzyloxy-6-methoxy-4-vinyl-pyridine-2-carboxylic acid methyl ester was treated in the hydrolysis condition as described above with lithium hydroxide to provide 3-benzyloxy-6-methoxy-4-vinyl-pyridine-2-carboxylic acid (40.6 mg, 99%) as a colorless oil.

¹H NMR (400 MHz, CDCl₃): δ [ppm] 7.53 (m, 2H), 7.41 (m, 3H), 7.11 (s, 1H), 6.94 (m, 1H), 5.98 (d, 1H), 5.57 (d, 1H), 5.01 (s, 2H), 4.01 (s, 3H).

Step III

3-Benzyloxy-6-methoxy-4-vinyl-pyridine-2-carboxylic acid was treated in the amidation condition as described above with 4-fluorobenzylamide to provide 3-benzyloxy-6-methoxy-4-vinyl-pyridine-2-carboxylic acid 4-fluoro-benzylamide (38.5 mg, 69%) as a white solid.

¹H NMR (400 MHz, CDCl₃): δ [ppm] 7.93 (br s, 1H), 7.49 (m, 2H), 7.39-7.29 (m, 5H), 7.01 (m, 3H), 6.94 (m, 1H), 5.88 (d, 1H), 5.45 (d, 1H), 4.99 (s, 2H), 4.61 (d, 2H), 3.90 (s, 3H).

Step IV

3-Benzyloxy-6-methoxy-4-vinyl-pyridine-2-carboxylic acid 4-fluoro-benzylamide was treated in the deprotection condition as described above with TMSI to provide 3-hydroxy-6-methoxy-4-vinyl-pyridine-2-carboxylic acid 4-fluoro-benzylamide (13.3 mg, 69%) as a colorless oil.

¹H NMR (400 MHz, CDCl₃): δ [ppm] 12.18 (s, 1H), 8.05 (br s, 1H), 7.32 (m, 2H), 7.06-6.91 (m, 4H), 6.01 (d, 2H), 5.53 (d, 1H), 4.60 (d, 2H), 3.83 (s, 3H).

### 6-(4-Fluoro-benzylcarbamoyl)-3-hydroxy-4-methoxy-pyridine-2-carboxylic acid methyl ester compound 78

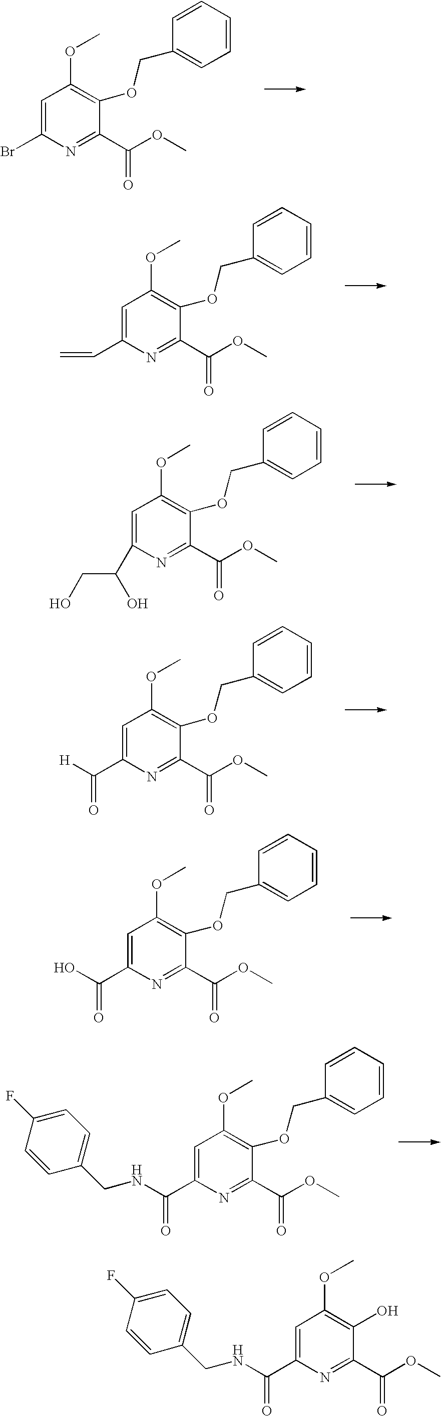 Step I

Compound 3-benzyloxy-6-bromo-4-methoxy-pyridine-2-carboxylic acid methyl ester was subjected to the Stille coupling reaction to prepare its corresponding vinyl analogue, which was further derivatized by the dihydroxylation reaction. The resulting diol was treated with sodium periodate to provide the related aldehyde, which was oxidized to an acid. The amide coupling mediated by HATU generated the desired compound 3-benzyloxy-6-(4-fluoro-benzylcarbamoyl)-4-methoxy-pyridine-2-carboxylic acid methyl ester.

¹H NMR (400 MHz, CDCl₃): δ [ppm] 8.31 (br s, 1H), 7.92 (1s, 1H), 7.42 (m, 2H), 7.35 (m, 5H), 6.99 (m, 2H), 5.15 (s, 2H), 4.58 (d, 2H), 4.03 (s, 3H), 3.85 (s, 3H).

Step II

The benzyl protection group of 3-benzyloxy-6-(4-fluoro-benzylcarbamoyl)-4-methoxy-pyridine-2-carboxylic acid methyl ester was removed under catalytic hydrogenation to give the title compound.

¹H NMR (400 MHz, CDCl₃): δ [ppm] 11.10 (s, 1H), 8.20 (br s, 1H), 7.92 (1s, 1H), 7.34 (m, 2H), 6.99 (m, 2H), 4.62 (d, 2H), 4.03 (s, 3H), 4.01 (s, 3H).

LC/MS: m/z 335.1 (M+H⁺).

### 3-Hydroxy-4-methoxy-pyridine-2,6-dicarboxylic acid bis-(4-fluoro-benzylamide) compound 79

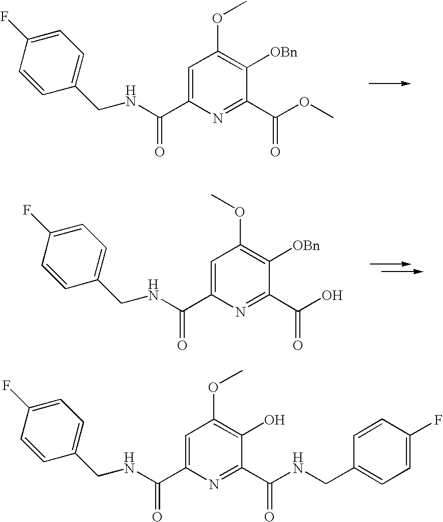

After hydrolysis of 3-benzyloxy-6-(4-fluoro-benzylcarbamoyl)-4-methoxy-pyridine-2-carboxylic acid methyl ester, the resulting acid was subjected to the amide coupling, and the obtained bis-amide was further deprotected by hydrogenolysis to yield the title compound.

¹H NMR (400 MHz, CDCl₃): δ [ppm] 12.85 (s, 1H), 8.19 (br s, 1H), 7.88 (brr s, 1H), 7.80 (1s, 1H), 7.23 (m, 2H), 6.99 (m, 2H), 4.75 (m, 4H), 3.98 (s, 3H).

LC/MS: m/z 428.3 (M+H⁺).

### (+)-3-hydroxy-4-methoxy-6-(tetrahydro-furan-2-yl)-pyridine-2-carboxylic acid 4-fluoro-benzylamide compound 42

The title enantiomer was obtained by chiral HPLC separation of the racemic mixture using a chiralcel OJ-RH column 4.6 mmID×150 mm eluted with 40% CH₃CN in H₂O (0.01M CH₃COONH₄) for 20 min at a flow rate of 1.0 mL/min.

tR=11.818 min,

[α]D=+43.2° (C=0.002, CH₃OH).

### (−)-3-hydroxy-4-methoxy-6-(tetrahydro-furan-2-yl)-pyridine-2-carboxylic acid 4-fluoro-benzylamide compound 43

The title enantiomer was obtained by chiral HPLC separation of the racemic mixture using a chiralcel OJ-RH column 4.6 mm Id×150 mm eluted with 40% CH₃CN in H₂O (0.01M CH₃COONH₄) for 20 min at a flow rate of 1.0 mL/min.

tR=15.269 min,

[α]D=−40.0° (C=0.002, CH₃OH).

### EXAMPLE 20

### List of Compounds

| | Structure | name |
|---|---|---|
| 1 | 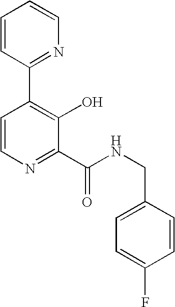 | 3′-Hydroxy- [2,4′]bipyridinyl-2′- carboxylic acid 4-fluoro- benzylamide |
| 2 | 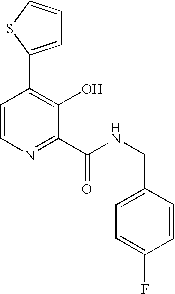 | 3-Hydroxy-4-thiophen-2-yl- pyridine-2-carboxylic acid 4-fluoro-benzylamide |
| 3 | 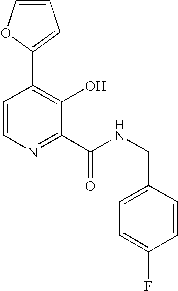 | 4-Furan-2-yl-3-hydroxy- pyridine-2- carboxylic acid 4-fluoro- benzylamide |
| 4 | 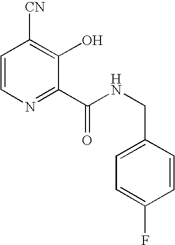 | 4-Cyano-3-hydroxy- pyridine-2-carboxylic acid 4-fluoro-benzylamide |
| 5 | 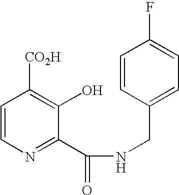 | 2-(4-Fluoro- benzylcarbamoyl)-3- hydroxy-isonicotinic acid |
| 6 | 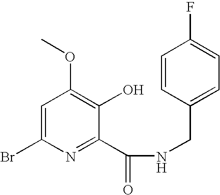 | 6-Bromo-3-hydroxy-4- methoxy-pyridine-2- carboxylic acid 4-fluoro- benzylamide |
| 7 | 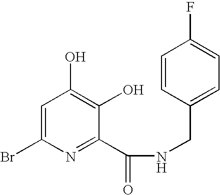 | 6-Bromo-3,4-dihydroxy- pyridine-2-carboxylic acid 4-fluoro-benzylamide |
| 8 | 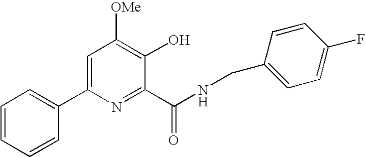 | 3-Hydroxy-4-methoxy-6- phenyl-pyridine-2- carboxylic acid 4-fluoro- benzylamide |
| 9 | 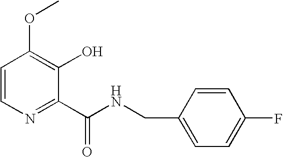 | 3-Hydroxy-4-methoxy- pyridine-2-carboxylic acid 4-fluoro-benzylamide |
| 10 | 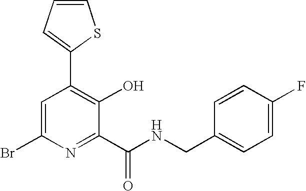 | 6-Bromo-3-hydroxy-4- thiophen-2-yl-pyridine-2- carboxylic acid 4-fluoro- benzylamide |
| 11 | 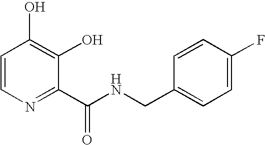 | 3,4-Dihydroxy-pyridine-2- carboxylic acid 4-fluoro-benzylamide |
| 12 | 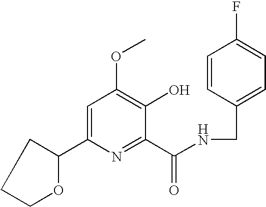 | 3-Hydroxy-4-methoxy-6- (tetrahydro-furan-2-yl)- pyridine-2-carboxylic acid 4-fluoro-benzylamide |
| 13 | 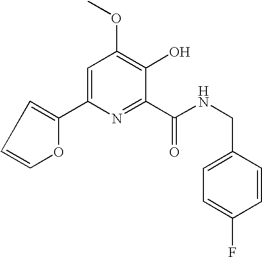 | 6-Furan-2-yl-3-hydroxy-4- methoxy- pyridine-2-carboxylic acid 4-fluoro-benzylamide |
| 14 | 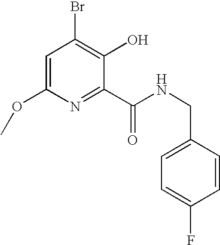 | 4-Bromo-3-hydroxy-6- methoxy-pyridine-2- carboxylic acid 4-fluoro- benzylamide |
| 15 | 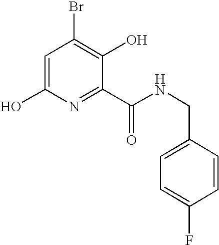 | 4-Bromo-3,6-dihydroxy- pyridine-2-carboxylic acid 4-fluoro-benzylamide |
| 16 | 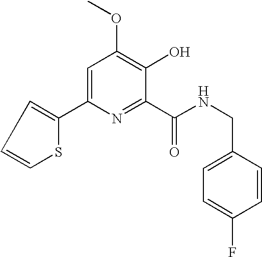 | 3-Hydroxy-4-methoxy-6- thiophen-2-yl- pyridine-2-carboxylic acid 4-fluoro-benzylamide |
| 17 | 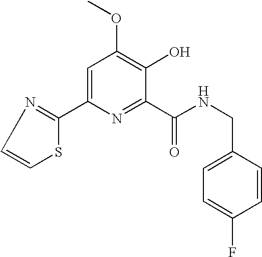 | 3-Hydroxy-4-methoxy-6- thiazol-2-yl- pyridine-2-carboxylic acid 4-fluoro-benzylamide |
| 18 | 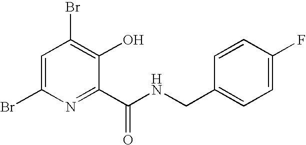 | 4,6-Dibromo-3-hydroxy- pyridine-2-carboxylic acid 4-fluoro-benzylamide |
| 19 | 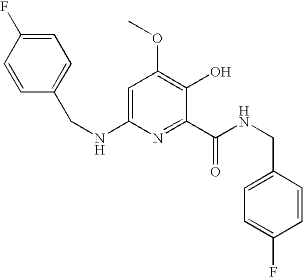 | 6-(4-Fluoro-benzylamino)- hydroxy- 4-methoxy-pyridine-2- carboxylic acid 4-fluoro- benzylamide |
| 20 | 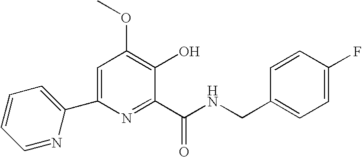 | 5-Hydroxy-4-methoxy- [2,2′]bipyridinyl-6- carboxylic acid 4-fluoro- benzylamide |
| 21 | 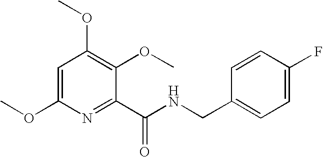 | 3,4,6-Trimethoxy-pyridine- 2-carboxylic acid 4- fluoro-benzylamide |
| 22 | 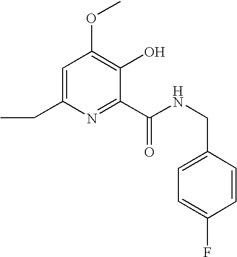 | 6-Ethyl-3-hydroxy-4- methoxy-pyridine-2- carboxylic acid 4-fluoro- benzylamide |
| 23 | 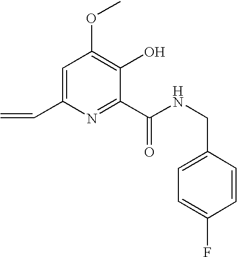 | 3-Hydroxy-4-methoxy-6- vinyl-pyridine-2- carboxylic acid 4-fluoro- benzylamide |
| 24 | 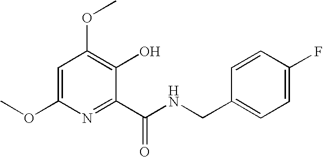 | 3-Hydroxy-4,6-dimethoxy- pyridine-2- carboxylic acid 4-fluoro- benzylamide |
| 25 | 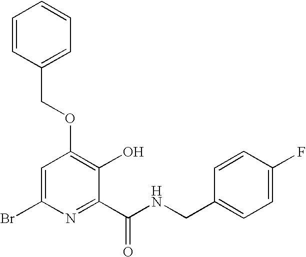 | 4-Benzyloxy-6-bromo-3- hydroxy-pyridine-2- carboxylic acid 4-fluoro- benzylamide |
| 26 | 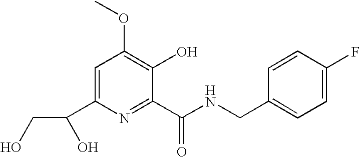 | 6-(1,2-Dihydroxy-ethyl)-3- hydroxy-4-methoxy- pyridine-2-carboxylic acid 4-fluoro-benzylamide |
| 27 | 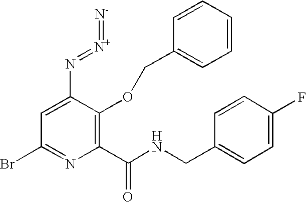 | 4-Azido-3-benzyloxy-6- bromo-pyridine-2- carboxylic acid 4-fluoro- benzylamide |
| 28 | 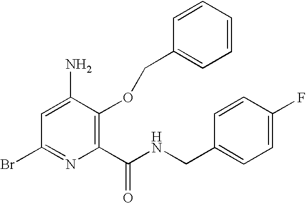 | 4-Amino-3-benzyloxy-6- bromo-pyridine-2- carboxylic acid 4-fluoro- benzylamide |
| 29 | 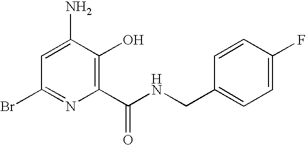 | 4-Amino-6-bromo-3-hydroxy- pyridine- 2-carboxylic acid 4- fluoro-benzylamide |
| 30 | 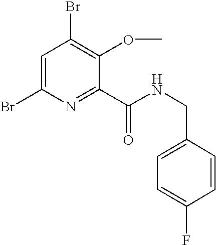 | 4,6-Dibromo-3-methoxy- pyridine-2-carboxylic acid 4-fluoro-benzylamide |
| 31 | 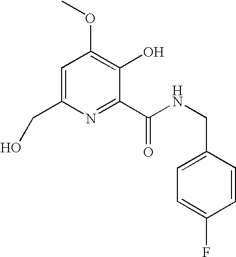 | 3-Hydroxy-6-hydroxymethyl- 4-methoxy-pyridine-2- carboxylic acid 4-fluoro- benzylamide |
| 32 | 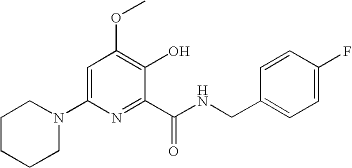 | 5′-Hydroxy-4′-methoxy- 3,4,5,6-tetra hydro-2H- [1,2′]bipyridinyl-6′- carboxylic acid 4-fluoro- benzylamide |
| 33 | 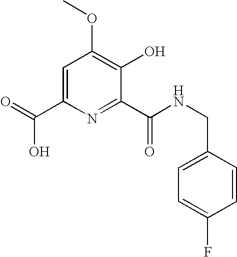 | 6-(4-Fluoro- benzylcarbamoyl)-5- hydroxy-4-methoxy- pyridine-2-carboxylic acid |
| 34 | 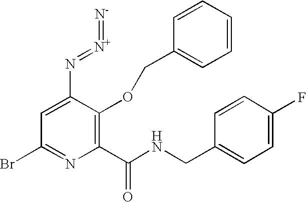 | 4-Azido-3-benzyloxy-6- bromo-pyridine-2- carboxylic acid 4-fluoro- benzylamide |
| 35 | 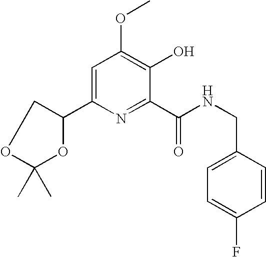 | 6-(2,2-Dimethyl- [1,3]dioxolan-4-yl)- 3-hydroxy-4-methoxy- pyridine-2-carboxylic acid 4-fluoro-benzylamide |
| 36 | 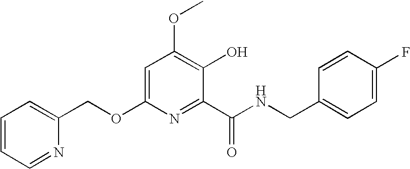 | 3-Hydroxy-4-methoxy-6- (pyridin-2-yl methoxy)-pyridine-2- carboxylic acid 4-fluoro-benzylamide |
| 37 | 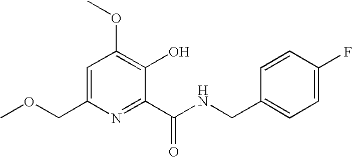 | 3-Hydroxy-4-methoxy-6- methoxymethyl-pyridine-2- carboxylic acid 4-fluoro- benzylamide |
| 38 | 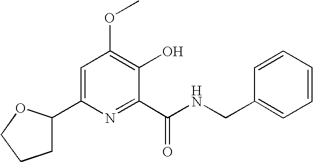 | 3-Hydroxy-4-methoxy-6- (tetrahydro-furan-2-yl)- pyridine-2-carboxylic acid benzylamide |
| 39 | 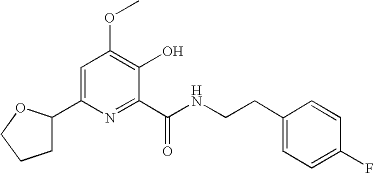 | 3-hydroxy-4-methoxy-6- (tetrahydro-furan-2-yl)- pyridine-2-carboxylic acid [2-(4-fluoro-phenyl)-ethyl]- amide |
| 40 | 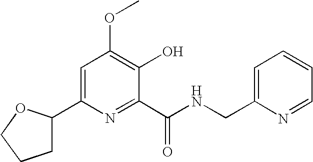 | 3-hydroxy-4-methoxy-6- (tetrahydro-furan-2-yl)- pyridine-2-carboxylic acid (pyridin-2-ylmethyl)-amide |
| 41 | 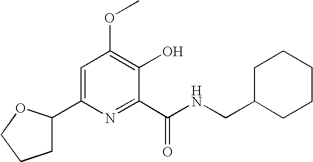 | 3-hydroxy-4-methoxy-6- (tetrahydro-furan-2-yl)- pyridine-2-carboxylic acid cyclohexylmethyl-amide |
| 42 | 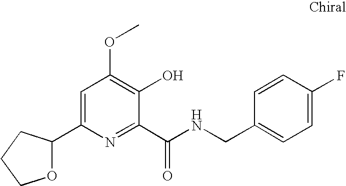 | (+)-3-hydroxy-4-methoxy-6- (tetrahydro-furan-2-yl)- pyridine-2-carboxylic acid 4- fluoro-benzylamide |
| 43 | 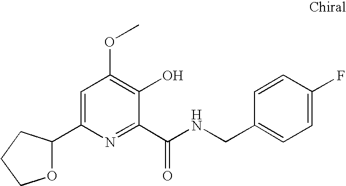 | (−)-3-hydroxy-4-methoxy-6- (tetrahydro-furan-2-yl)- pyridine-2-carboxylic acid 4- fluoro-benzylamide |
| 44 | 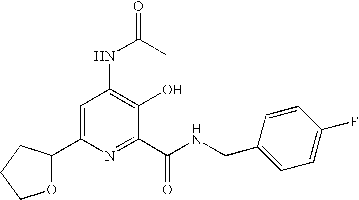 | 4-acetylamino-3-hydroxy-6- (tetrahydro-furan-2-yl)- pyridine-2-carboxylic acid 4- fluoro-benzylamide |
| 45 | 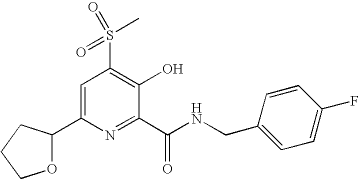 | 3-hydroxy-4-methanesulfonyl- 6-(tetrahydro-furan-2-yl)- pyridine-2-carboxylic acid 4- fluoro-benzylamide |
| 46 | 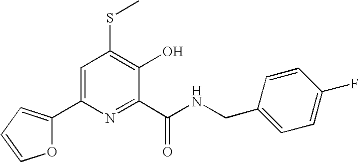 | 6-furan-2-yl-3-hydroxy-4- methylsulfanyl-pyridine-2- carboxylic acid 4-fluoro- benzylamide |
| 47 | 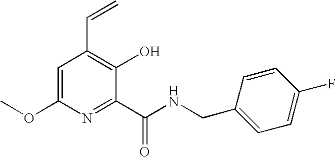 | 3-hydroxy-6-methoxy-4-vinyl- pyridine-2-carboxylic acid 4- fluoro-benzylamide |
| 48 | 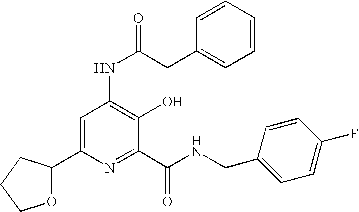 | 3-hydroxy-4- phenylacetylamino-6- (tetrahydro-furan-2-yl)- pyridine-2-carboxylic acid 4- fluoro-benzylamide |
| 49 | 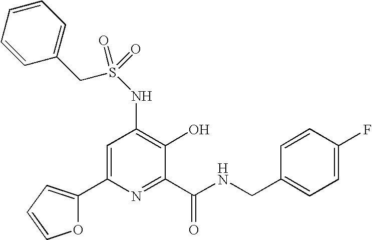 | 6-furan-2-yl-3-hydroxy-4- phenylmethanesulfonylamino- pyridine-2-carboxylic acid 4- fluoro-benzylamide |
| 50 | 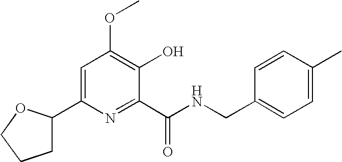 | 3-Hydroxy-4-methoxy-6- (tetrahydro-furan-2-yl)- pyridine-2-carboxylic acid 4- methyl-benzylamide |
| 51 | 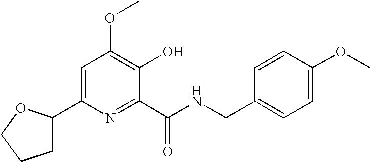 | 3-Hydroxy-4-methoxy-6- (tetrahydro-furan-2-yl)- pyridine-2-carboxylic acid 4- methoxy-benzylamide |
| 52 | 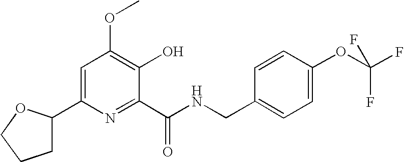 | 3-Hydroxy-4-methoxy-6- (tetrahydro-furan-2-yl)- pyridine-2-carboxylic acid 4- trifluoromethoxy-benzylamide |
| 53 | 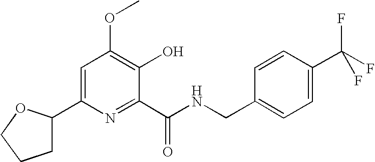 | 3-Hydroxy-4-methoxy-6- (tetrahydro-furan-2-yl)- pyridine-2-carboxylic acid 4- trifluoromethyl-benzylamide |
| 54 | 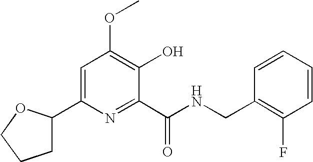 | 3-Hydroxy-4-methoxy-6- (tetrahydro-furan-2-yl)- pyridine-2-carboxylic acid 2- fluoro-benzylamide |
| 55 | 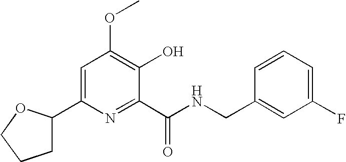 | 3-Hydroxy-4-methoxy-6- (tetrahydro-furan-2-yl)- pyridine-2-carboxylic acid 3- fluoro-benzylamide |
| 56 | 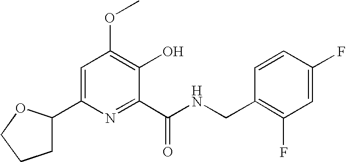 | 3-Hydroxy-4-methoxy-6- (tetrahydro-furan-2-yl)- pyridine-2-carboxylic acid 2,4-difluoro-benzylamide |
| 57 | 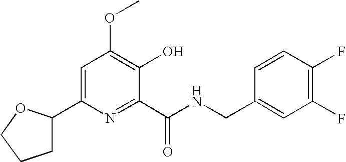 | 3-Hydroxy-4-methoxy-6- (tetrahydro-furan-2-yl)- pyridine-2-carboxylic acid 3,4-difluoro-benzylamide |
| 58 | 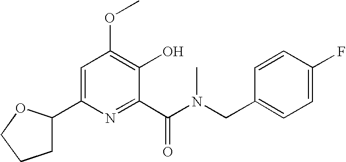 | 3-hydroxy-4-methoxy-6- (tetrahydro-furan-2-yl)- pyridine-2-carboxylic acid (4-fluoro-benzyl)-methyl- amide |
| 59 | 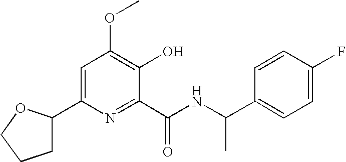 | 3-Hydroxy-4-methoxy-6- (tetrahydro-furan-2-yl)- pyridine-2-carboxylic acid [1-(4-fluoro-phenyl)-ethyl]- amide |
| 60 | 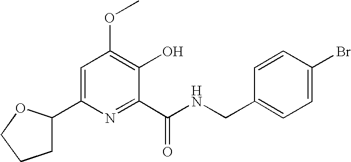 | 3-Hydroxy-4-methoxy-6- (tetrahydro-furan-2-yl)- pyridine-2-carboxylic acid 4- bromo-benzylamide |
| 61 | 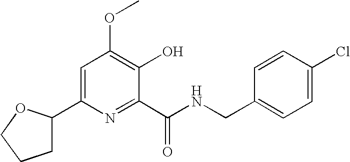 | 3-Hydroxy-4-methoxy-6- (tetrahydro-furan-2-yl)- pyridine-2-carboxylic acid 4- chloro-benzylamide |
| 62 | 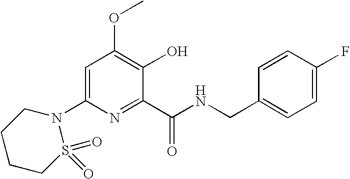 | 6-(1,1-Dioxo-[1,2]-thiazinan- 2-yl)-3-hydroxy-4-methoxy- pyridine-2-carboxylic acid 4- fluoro-benzylamide |
| 63 | 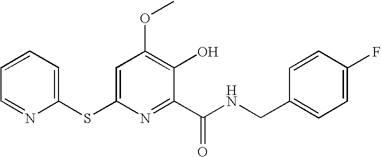 | 3-Hydroxy-4-methoxy-6- (pyridin-2-yl-sulfanyl)- pyridine-2-carboxylic acid 4- fluoro-benzylamide |
| 64 | 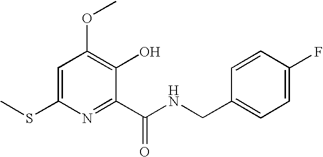 | 3-Hydroxy-4-methoxy-6- methylsulfanyl-pyridine-2- carboxylic acid 4-fluoro- benzylamide |
| 65 | 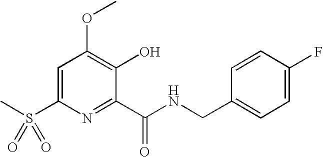 | 3-Hydroxy-6-methanesulfonyl- 4-methoxy pyridine-2- carboxylic acid 4-fluoro- benzylamide |
| 66 | 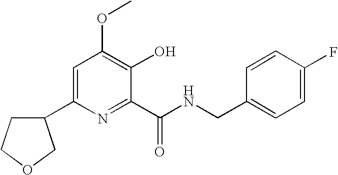 | 3-Hydroxy-4-methoxy-6- (tetrahydrofuran-3-yl)- pyridine-2-carboxylic acid 4- fluoro-benzylamide |
| 67 | 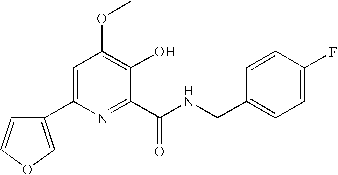 | 6-Furan-3-yl-3-hydroxy-4- methoxy-pyridine-2-carboxylic acid 4-fluoro-benzylamide |
| 68 | 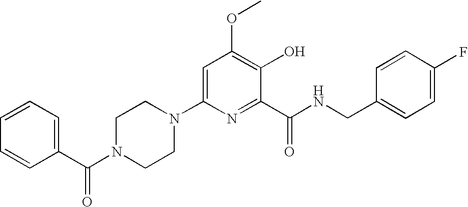 | 6-(4-Benzoyl-piperazin-1-yl)- 3-hydroxy-4-methoxy-pyridine- 2-carboxylic acid 4-fluoro- benzylamide |
| 69 | 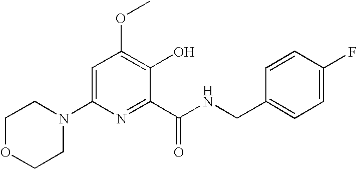 | 3-Hydroxy-4-methoxy-6- morpholin-4-yl-pyridine-2- carboxylic acid 4-fluoro- benzylamide |
| 70 | 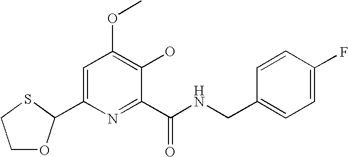 | 3-Hydroxy-4-methoxy-6-(1,3)- oxathioan-2-yl-pyridine-2- carboxylic acid 4-fluoro- benzylamide |
| 71 | 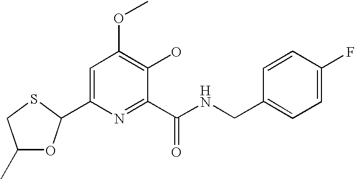 | 3-Hydroxy-4-methoxy-6-(5- methyl-(1,3)-oxathioan-2-yl)- pyridine-2-carboxylic acid 4- fluoro-benzylamide |
| 72 | 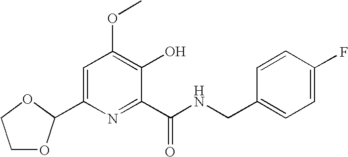 | 6-(1,3)-Dioxolan-2-yl-3- hydroxy-4-methoxy-pyridine-2- carboxylic acid 4-fluoro- benzylamide |
| 73 | 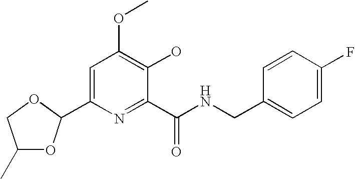 | 3-Hydroxy-4-methoxy-6-(4- methyl-(1,3)dioxolan-2-yl)- pyridine-2-carboxylic acid 4- fluoro-benzylamide |
| 74 | 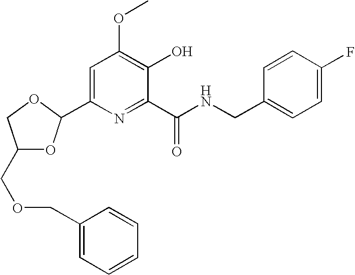 | 6-(4-Benzyloxymethyl-(1,3)- dioxolan-2-yl)-3-hydroxy-4- methoxy-pyridine-2-carboxylic acid 4-fluoro-benzylamide |
| 75 | 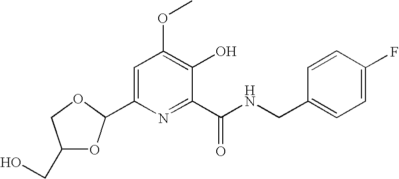 | 3-Hydroxy-6-(4-hydroxymethyl- (1,3)-dioxolan-2-yl)-4- methoxy-pyridine-2-carboxylic acid 4-fluoro-benzylamide |
| 76 | 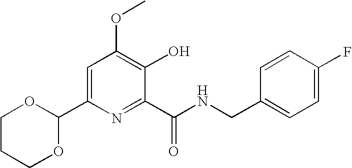 | 6-(1,3)-Dioxan-2-yl-3- hydroxy-4-methoxy-pyridine-2- carboxylic acid 4-fluoro- benzylamide |
| 77 | 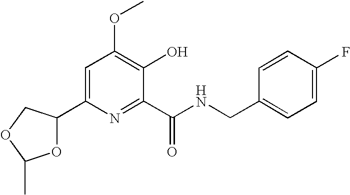 | 3-Hydroxy-4-methoxy-6-(2- methyl-(1,3)-dioxolan-2-yl)- pyridine-2-carboxylic acid 4- fluoro-benzylamide |
| 78 | 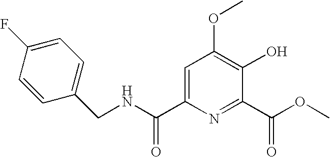 | 6-(4-Fluoro-benzylcarbamoyl)- 3-hydroxy-4-methoxy-pyridine- 2-carboxylic acid methyl ester |
| 79 | 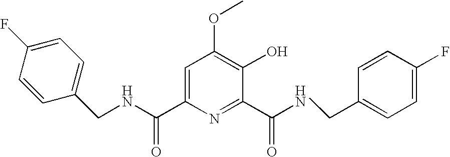 | 3-Hydroxy-4-methoxy-pyridine- 2,6-dicarboxylic acid bis-(4- fluoro-benzylamide) |
| 80 | 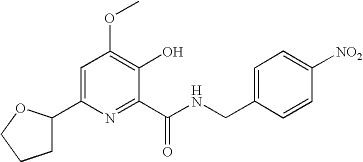 | 3-Hydroxy-4-methoxy-6- (tetrahydro-furan-2-yl)- pyridine-2-carboxylic acid 4- nitro-benzylamide |
| 81 | 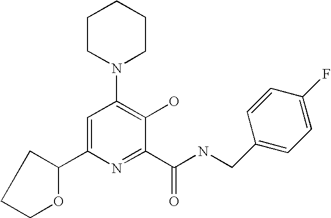 | 3′-Hydroxy-6′-(tetrahydro- furan-2-yl)-3,4,5,6- tetrahydro-2H- (1,4′)bipyridinyl- 2′carboxylic acid 4-fluoro- benzylamide |

### EXAMPLE 21

HIV Integrase Strand Transfer Inhibition Assay

Methods for evaluating biological activity of HIV and HIV integrase inhibitors are described in: PNAS (2002) vol. 19 number 10, pages 6661-6666 “Diketo acid inhibitor mechanism and HIV-1 integrase: Implications for metal binding in the active site of phosphotransferase enzymes” ‘Grobler, J. A. et al.

### EXAMPLE 22

Anti-HIV-1 Replication Assay in H9 Cells for Anti-HIV-1 Integrase Compounds.

The anti-HIV-1 activities of the compounds were tested by employing HIV-1IIIB in H9 cells. The prepared cells were suspended at 5×106/ml in complete medium (RPMI 1640, 10% FBS, 2 mM glutamine, 100 units penicillin/ml, 100 μg streptomycin/ml), incubated with virus at a multiplicity of infection of 0.1 for 2 h in an atmosphere of 5% CO₂ and 37° C. The infected cells were washed twice with PBS to remove residual virus and cultured at presence of inhibitors at serial concentrations for 7-8 days. The anti-HIV-1 efficacy was determined by testing for HIV-1 RT activity in the cell culture supernatants. All assays were performed in duplicate with Merck compound L-731988 and Shionogi compound S-1360 as control. The 50% effective concentrations (IC50s) were calculated from the linear portion of the dose-response curve.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound according to formula I 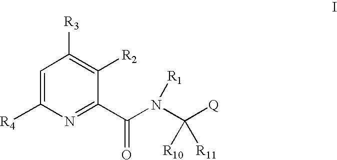
or a pharmaceutically acceptable salt thereof,
wherein,
R₁ is hydrogen or C₁₋₁₀ alkyl;
R₂ is hydroxyl, C₁₋₁₀ alkoxy or C₆aryl-C₁₋₁₀ alkyloxy;
R₃ is amino, amido, sulfonamido, azido, hydroxyl, halogen, cyano, carboxy, C₁₋₁₀ alkoxy, 5-6 membered heterocycle, C₆₋₁₀ aryl-C₁₋₁₀ alkyloxy, C₁₋₁₀ alkyl, or SOₙR₁₂;
n is 0, 1, or 2;
R₄ is hydrogen, halogen, hydroxyl, carboxy, C₁₋₁₀ alkyl, amino, amido, sulfonamide, SOₙR₁₂, C₁₋₁₀ alkoxy, C₆₋₁₀ aryl, 5-6 membered heterocycle, or C₅₋₁₀ heteroaryl;
R₁₀, R₁₁, R₁₂ are each independently hydrogen, C₁₋₁₀ alkyl, C₆₋₁₀ aryl, or C₇₋₁₂aralkyl;
Q is optionally substituted phenyl, C₁₋₁₀ alkyl, 5-6 membered heterocycle, or C₇₋₁₂aralky;
with the proviso that when R₃ is methoxy, R₂ is hydroxyl, R₁ is hydrogen and R₄ is hydrogen then Q is phenyl substituted by at least 3 substituents;
wherein said alkyl, alkoxy, aryl, 5-6 membered heterocycle, heteroaryl, aralkyl, and phenyl groups are, in each case, independently optionally substituted one or more times by halogen, amino, amidino, amido, azido, cyano, guanidino, hydroxyl, nitro, nitroso, urea, OS(O)₂Rₘ, OS(O)₂ORₙ, S(O)₂ORₚ, S(O)₀₋₂R_{q}, OP(O)ORₛORₜ, P(O)ORₛORₜ, C₁₋₁₀alkyl, C₆aryl-C₁₋₁₀alkyl, C₆₋₁₀aryl, C₁₋₁₀alkoxy, C₆aryl-C₁₋₁₀alkyloxy, C₆₋₁₀aryloxy, 3-10 membered heterocycle, C(O)Rᵤ, C(O)ORᵥ, NRₓC(O)R_{w} or SO₂NR_{y}R_{z};
Rₘ is C₁₋₁₀ alkyl, C₆₋₁₀ aryl or 3-10 membered heterocycle;
Rₙ is H, C₁₋₁₀ alkyl, C₆₋₁₀ aryl or 3-10 membered heterocycle;
Rₚ is H, C₁₋₁₀ alkyl, C₆₋₁₀ aryl or 3-10 membered heterocycle;
R_{q} is H, C₁₋₁₀ alkyl, C₆₋₁₀ aryl or 3-10 membered heterocycle;
Rₛ and Rₜ are each independently H or C₁₋₁₀ alkyl;
Rᵤ is H, C₁₋₁₀ alkyl, C₆₋₁₀ aryl, C₆₋₁₂ aralkyl or 3-10 membered heterocycle;
Rᵥ is H, C₁₋₁₀ alkyl, C₆₋₁₀ aryl, C₆aryl-C₁₋₁₀alkyl or 3-10 membered heterocycle;
Rₓ is H or C₁₋₁₀ alkyl and R_{w} is H, C₁₋₁₀ alkyl, C₆₋₁₀ aryl, C₆aryl-C₁₋₁₀alkyl or 3-10 membered heterocycle, or Rₓ and R_{w} are taken together with the atoms to which they are attached to form a 3-10 membered heterocycle;
R_{y} and R_{z} are each independently H, C₁₋₁₀ alkyl, C₆₋₁₀ aryl, 3-10 membered heterocycle or C₆aryl-C₁₋₁₀alkyl);
amidino is —C(═NRₐ)NR_{b}R_{c} wherein Rₐ, R_{b} and R_{c} are each independently H, C₁₋₁₀ alkyl, C₆₋₁₂ aryl or C₆₋₁₂ aralkyl, or R_{b} and R_{c} are taken together with the nitrogen to which they are attached to form a 3 to 10 membered heterocycle;
guanidine is —N(R_{d})C(═NRₑ)NR_{f}R_{g} wherein R_{d}, Rₑ, R_{f} and R_{g} are each independently H, C₁₋₁₀ alkyl, C₆₋₁₂ aryl or C₆₋₁₂ aralkyl, or R_{f} and R_{g} are taken together with the nitrogen to which they are attached to form a 3 to 10 membered heterocycle;
amido is —CONH₂, —CONHRₕ, —CONRₕRᵢ, —NHCORₕ or —NRₕCORᵢ, wherein Rₕ and Rₗ are each independently C₁₋₁₀ alkyl, C₆₋₁₂ aryl or C₆₋₁₂ aralkyl, or Rₕ and Rᵢ are taken together with the nitrogen to which they are attached to form a 3 to 10 membered heterocycle;
amino is —NH₂, —NHRⱼ and —NRⱼRₖ, wherein Rⱼ and Rₖ are each independently C₁₋₁₀ alkyl, C₆₋₁₂ aryl or C₆₋₁₂ aralkyl, or Rⱼ and Rₖ are taken together with the nitrogen to which they are attached to form a 3 to 10 membered heterocycle;
sulfonamido is —SO₂NH₂, —SO₂NHR_{L}, —SO₂NR_{L}R_{LL}, and —NR_{L}SO₂R_{LL}, wherein R_{L} and R_{LL} are each independently C₁₋₁₀ alkyl, C₆₋₁₂ aryl or C₆₋₁₂ aralkyl, or R_{L} and R_{LL} are taken together with the nitrogen to which they are attached to form a 3 to 10 membered heterocycle; and
urea is —N(Rₐₐ)CONR_{bb}R_{cc} wherein Rₐₐ is H or C₁₋₁₀ alkyl and wherein R_{bb} and R_{cc} are each independently the group consisting of H, C₁₋₁₀ alkyl, C₆₋₁₀ aryl, 3-10 membered heterocycle, or C₆₋₁₂ aralkyl, or R_{bb} and R_{cc} are taken together with the nitrogen to which they are attached to form a C₃₋₁₀ heterocycle.

2. A compound according to claim 1, wherein:
R₁ is hydrogen or C₁₋₆ alkyl;
R₂ is hydroxyl, C₁₋₆ alkoxy or C₆aryl-C₁₋₆ alkyloxy;
R₃ is amino, azido, hydroxyl, halogen, cyano, carboxy, C₁₋₆ alkoxy, 5-6 membered heterocycle, or C₆aryl-C₁₋₆ alkyloxy;
R₄ is hydrogen, halogen, hydroxyl, carboxy, C₁₋₆ alkyl, C₁₋₆ alkoxy, 5-6 membered heterocycle, or C₆₋₁₀ aryl;
R₁₀, R₁₁, R₁₂ are each independently hydrogen or C₁₋₁₀ alkyl; and
Q is optionally substituted phenyl;
with the proviso that when R₃ is methoxy, R₂ is hydroxyl, R₁ is hydrogen and R₄ is hydrogen then Q is phenyl substituted by at least 3 substituents.

3. A compound according to claim 1, wherein R₁ is hydrogen or C₁₋₁₀ alkyl.

4. A compound according to claim 1, wherein R₁ is methyl, ethyl, propyl, isopropyl, cyclopropyl or cyclohexyl.

5. A compound according to claim 1, wherein R₂ is hydroxyl, C₁₋₁₀alkoxy or C₆aryl-C₁₋₁₀ alkyloxy.

6. A compound according to claim 1, wherein R₂ is hydroxyl or C₁₋₁₀ alkoxy.

7. A compound according to claim 1, wherein R₂ is C₁₋₃ alkoxy.

8. A compound according to claim 1, wherein R₂ is methoxy, ethyloxy, propyloxy, isopropyloxy, cyclopropyloxy or cyclohexyloxy.

9. A compound according to claim 1, wherein R₂ is methoxy or benzyloxy.

10. A compound according to claim 1, wherein R₃ is amino, amido, sulfonamido, azido, hydroxyl, halogen, cyano, carboxyl, C₁₋₁₀ alkoxy, 5-6 membered heterocycle, C₆aryl-C₁₋₁₀ alkyloxy, C₁₀ alkyl, or SOₙR₁₂.

11. A compound according to claim 1, wherein R₃ is hydroxyl, halogen, C₁₋₁₀ alkoxy or 5-6 membered heterocycle.

12. A compound according to, claim 1, wherein R₃ is methoxy, ethyloxy, propyloxy, isopropyloxy, cyclopropyloxy and cyclohexyloxy.

13. A compound according to claim 1, wherein R₃ is methoxy, amino, azido, hydroxyl, halogen, cyano, carboxy, amido, sulfonamide, or SOₙR₁₂.

14. A compound according to claim 1, wherein R₃ is pyridinyl, thiazolyl, furanyl, thienyl or piperidinyl.

15. A compound according to claim 1, wherein R₃ is 2-pyridinyl, 2-thiazolyl, 2-furanyl, 2-thienyl or 1-piperidinyl.

16. A compound according to claim 1, wherein R₃ is benzyloxy.

17. A compound according to claim 1, wherein R₄ is hydrogen, halogen, hydroxyl, carboxy, C₁₋₁₀alkyl, amino, amido, sulfonamide, SOₙR₁₂, C₁₋₁₀ alkoxy, 5-6 membered heterocycle, or C₅₀ heteroaryl.

18. A compound according to claim 1, wherein R₄ is halogen, C₁₋₁₀ alkyl, C₁₋₁₀ alkoxy or 5-6 membered heterocycle.

19. A compound according to claim 1, wherein R₄ is C₁₋₃ alkyl.

20. A compound according to claim 1, wherein R₄ is methyl, ethyl, propyl, isopropyl, vinyl, 1,2-dihydroxyethyl, hydroxymethyl, methyloxymethyl, cyclopropyl or cyclohexyl;

21. A compound according to claim 1, wherein R₄ is methyl, ethyl, vinyl, 1,2-dihydroxyethyl, hydroxymethyl or methyloxymethyl.

22. A compound according to claim 1, wherein R₄ is hydroxyl, carboxy, aryl, amino, amido, sulfonamide, SOₙR₁₂, or C₁₋₁₀ alkoxy.

23. A compound according to claim 1, wherein R₄ is methoxy, ethyloxy, propyloxy, isopropyloxy, cyclopropyloxy or cyclohexyloxy.

24. A compound according to claim 1, wherein R₄ is 5-6 membered heterocycle.

25. A compound according to claim 1, wherein R₄ is furanyl, tetrahydrofuranyl, thienyl, thiazolyl, pyridinyl, 2,2-dimethyl[1,3]dioxolanyl or piperidinyl.

26. A compound according to claim 1, wherein R₁₀ and R₁₁ are each independently hydrogen or C₁₋₁₀ alkyl.

27. A compound according to claim 1, wherein R₁₀ and R₁₁ are each C₁₋₁₀ alkyl.

28. A compound according to claim 1, wherein R₁₀ is hydrogen and R₁₁ is C₁₋₁₀ alkyl.

29. A compound according to claim 1, wherein R₁₀ is hydrogen and R₁₁ is methyl.

30. A compound according to claim 1, wherein R₁₀ and R₁₁ are each C₁₋₃ alkyl.

31. A compound according to claim 1, wherein Q is optionally substituted phenyl, C₁₋₁₀ alkyl, 5-6 membered heterocycle or C₇₋₁₂aralkyl.

32. A compound according to claim 1, wherein Q is C₁₋₁₀ alkyl, cyclohexyl, 5-6 membered heterocycle, 2-pyridinyl, C₇₋₁₂aralkyl, benzyl, or phenyl.

33. A compound according to claim 1, wherein
Q is phenyl substituted by one or more substituents independently selected from halogen, amino, amidino, amido, azido, cyano, guanidino, hydroxyl, nitro, nitroso, urea, OS(O)₂Rₘ, OS(O)₂ORₙ, S(O)₂ORₚ, S(O)₀₋₂R_{q}, OP(O)ORₛORₜ, P(O)OR₅ORₜ, C(O)ORᵥ, NRₓC(O)R_{w} or SO₂NR_{y}R_{z};
Rₘ is C₁₋₁₀ alkyl, C₆₋₁₀ aryl or 3-10 membered heterocycle;
Rₙ is H, C₁₀ alkyl, C₆₋₁₀ aryl or 3-10 membered heterocycle;
Rₚ is H, C₁₋₁₀ alkyl, C₆₋₁₀ aryl or 3-10 membered heterocycle;
R_{q} is H, C₁₋₁₀ alkyl, C₆₋₁₀ aryl or 3-10 membered heterocycle;
Rₛ and Rₜ are each independently H or C₁₋₁₀ alkyl, C₁₋₁₀alkyl, C₆₋₁₂aralkyl, C₆₋₁₀aryl, C₁₋₁₀alkoxy, C₆₋₁₂aralkyloxy, C₆₋₁₀aryloxy, 3-10 membered heterocycle, or C(O)Rᵤ;
Rᵤ is H, C₁₋₁₀ alkyl, C₆₋₁₀ aryl, C₆₋₁₂ aralkyl or 3-10 membered heterocycle, or C(O)ORᵥ;
Rᵥ is H, C₁₋₁₀ alkyl, C₆₋₁₀ aryl, C₆₋₁₂ aralkyl or 3-10 membered heterocycle,
Rₓ is H or C₁₋₁₀alkyl, and R_{w} is H, C₁₋₁₀ alkyl, C₆₋₁₀ aryl, C₆₋₁₂ aralkyl (or 3-10 membered heterocycle, or Rₓ and R_{w}, taken together with the atoms to which they are attached, form a 3 to 10 membered heterocycle; and
R_{y} and R_{z} are each independently H, C₁₋₁₀ alkyl, C₆₋₁₀ aryl, C₃₋₁₀ heterocycle or C₆₋₁₂ aralkyl.

34. A compound according to claim 34, wherein
Rₛ and Rₜ are each independently H or C₁₋₁₀ alkyl, C₁₋₁₀ alkyl, C₇₋₁₂aralkyl, C₆₋₁₀aryl, C₁₋₁₀alkoxy, C₇₋₁₂aralkyloxy, C₆₋₁₀aryloxy, 3-10 membered heterocycle, or C(O)Rᵤ.

35. A compound according to claim 1, wherein
Q is phenyl substituted by one or more substituents independently selected from halogen, amino, amido, azido, cyano, hydroxyl, urea, S(O)₂ORₚ, S(O)₂R_{q}, P(O)ORₛORₜ, C₁₋₁₀alkyl, C₁₋₁₀alkoxy, C(O)Rᵤ, C(O)ORᵥ, NRₓC(O)R_{w} and SO₂NR_{y}R_{z};
Rₚ is H or C₁₋₁₀ alkyl
R_{q} is H or C₁₋₁₀ alkyl;
Rₛ and Rₜ are each independently H or C₁₋₁₀ alkyl;
Rᵤ is H or C₁₋₁₀ alkyl
Rᵥ is H, or C₁₋₁₀ alkyl;
Rₓ is H or C₁₋₁₀ alkyl;
R_{w} is H or C₁₋₁₀ alkyl; and
R_{y} and R_{z} are each independently H or C₁₋₁₀ alkyl.

36. A compound according to claim 1, wherein
Q is phenyl substituted by one or more substituents independently selected from halogen, amino, amido, azido, cyano, hydroxyl, C₁₋₁₀alkyl, C₁₋₁₀alkoxy, C(O)Rᵤ, C(O)ORᵥ, and SO₂NR_{y}R_{z};
Rᵤ is H or C₁₋₁₀ alkyl;
Rᵥ is H, or C₁₋₁₀ alkyl; and
R_{y} and R_{z} are each independently H or C₁₋₁₀ alkyl

37. A compound according to claim 1, wherein
Q is phenyl substituted by one or more substituents independently selected from halogen, amino, amido, cyano, hydroxyl, C₁₋₁₀alkyl, C₁₋₁₀alkoxy, C(O)Rᵤ, C(O)ORᵥ, and SO₂NR_{y}R_{z};
Rᵤ is H or C₁₋₁₀ alkyl;
Rᵥ is H, or C₁₋₁₀ alkyl; and
R_{y} and R_{z} are each independently H or C₁₋₁₀ alkyl

38. A compound according to claim 1, wherein Q is 4-fluorophenyl.

39. A compound according to claim 1, wherein:
R₁ is hydrogen or C₁₋₁₀ alkyl;
R₂ is hydroxyl, C₁₋₁₀ alkoxy or C₆aryl-C₁₋₁₀ alkyloxy;
R₃ is amino, amido, sulfonamido, azido, hydroxyl, halogen, cyano, carboxy, C₁₋₁₀ alkoxy, 5-6 membered heterocycle, C₆aryl-C₁₋₁₀ alkyloxy, C₁₋₁₀ alkyl, or SOₙR₁₂;
n is 0, 1, or 2;
R₄ is hydrogen, halogen, hydroxyl, carboxy, C₁₋₁₀ alkyl, amino, amido, sulfonamide, SOₙR₁₂, C₁₋₁₀ alkoxy, C₆₋₁₀ aryl, 5-6 membered heterocycle, or C₅₋₁₀ heteroaryl;
R₁₀ and R₁₁ are each independently selected from hydrogen or C₁₋₁₀ alkyl; and
Q is a phenyl optionally substituted, C₁₋₁₀ alkyl, 5-6 membered heterocycle, or C₇₋₁₂aralkyl.

40. A compound according to claim 1, wherein:
R₁ is hydrogen or C₁₋₆ alkyl;
R₂ is hydroxyl, C₁₋₆ alkoxy or C₆aryl-C₁₋₆ alkyloxy;
R₃ is amino, azido, hydroxyl, halogen, cyano, carboxy, C₁₋₆ alkoxy, 5-6 membered heterocycle, or C₆aryl-C₁₋₆ alkyloxy;
R₄ is halogen, hydroxyl, carboxy, C₁₋₆ alkyl, C₁₋₆ alkoxy, 5-6 membered heterocycle, or C₆₋₁₀ aryl;
R₁₀ and R₁₁ are each independently selected from hydrogen or C₁₋₆ alkyl;
Q is optionally substituted phenyl.

41. A compound according to claim 1, wherein said compound is a compound of formula II: 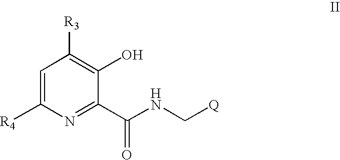
or a pharmaceutically acceptable salt thereof wherein,
R₃ is amino, amido, sulfonamido, azido, hydroxyl, halogen, cyano, carboxy, C₁₋₁₀ alkoxy, 5-6 membered heterocycle, C₆aryl-C₁₋₁₀ alkyloxy, or C₁₋₁₀ alkyl, or SOₙR₁₂;
n is 0, 1, or 2;
R₄ is hydrogen, halogen, hydroxyl, carboxy, C₁₋₁₀ alkyl, amino, amido, sulfonamide, SOₙR₁₂, C₁₋₁₀ alkoxy, C₆₋₁₀ aryl, 5-6 membered heterocycle, or C₅₋₁₀ heteroaryl; and
Q is optionally substituted phenyl, C₁₋₁₀ alkyl, 5-6 membered heterocycle, or C₁₋₁₂aralkyl.

42. A compound according to claim 41, wherein:
R₃ is amino, azido, hydroxyl, halogen, cyano, carboxy, C₁₋₆ alkoxy, 5-6 membered heterocycle, or C₆aryl-C₁₋₆ alkyloxy;
R₄ is hydrogen, halogen, hydroxyl, carboxy, C₁₋₆ alkyl, C₁₋₆ alkoxy, 5-6 membered heterocycle, or C₆₋₁₀ aryl;
Q is optionally substituted phenyl.

43. A compound according to claim 1, wherein said compound is a compound of formula III: 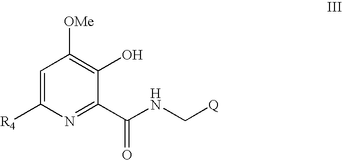
or a pharmaceutically acceptable salt thereof,
wherein
R₄ is hydrogen, halogen, hydroxyl, carboxy, C₁₋₁₀ alkyl, amino, amido, sulfonamide, SOₙR₁₂, C₁₋₁₀ alkoxy, C₆₋₁₀ aryl, 5-6 membered heterocycle, or C₅₋₁₀ heteroaryl; and
Q is a phenyl optionally substituted, C₁₋₁₀ alkyl, 5-6 membered heterocycle, or C₇₋₁₂aralkyl.

44. A compound according to claim 43, R₄ is halogen, hydroxyl, carboxy, C₁₋₆ alkyl, C₁₋₆ alkoxy, 5-6 membered heterocycle, or C₆₋₁₀ aryl; and Q is a phenyl optionally substituted.

45. A compound according to claim 1, wherein said compound is selected from:
3′-Hydroxy-[2,4′]bipyridinyl-2′-carboxylic acid 4-fluoro-benzylamide;
3-Hydroxy-4-thiophen-2-yl-pyridine-2-carboxylic acid 4-fluoro-benzylamide;
4-Furan-2-yl-3-hydroxy-pyridine-2-carboxylic acid 4-fluoro-benzylamide;
4-Cyano-3-hydroxy-pyridine-2-carboxylic acid 4-fluoro-benzylamide;
2-(4-Fluoro-benzylcarbamoyl)-3-hydroxy-isonicotinic acid;
6-Bromo-3-hydroxy-4-methoxy-pyridine-2-carboxylic acid 4-fluoro-benzylamide;
6-Bromo-3,4-dihydroxy-pyridine-2-carboxylic acid 4-fluoro-benzylamide;
3-Hydroxy-4-methoxy-6-phenyl-pyridine-2-carboxylic acid 4-fluoro-benzylamide;
3-Hydroxy-4-methoxy-pyridine-2-carboxylic acid 4-fluoro-benzylamide;
6-Bromo-3-hydroxy-4-thiophen-2-yl-pyridine-2-carboxylic acid 4-fluoro-benzylamide;
3,4-Dihydroxy-pyridine-2-carboxylic acid 4-fluoro-benzylamide;
3-Hydroxy-4-methoxy-6-(tetrahydro-furan-2-yl)-pyridine-2-carboxylic acid 4-fluoro-benzylamide;
6-Furan-2-yl-3-hydroxy-4-methoxy-pyridine-2-carboxylic acid 4-fluoro-benzylamide;
4-Bromo-3-hydroxy-6-methoxy-pyridine-2-carboxylic acid 4-fluoro-benzylamide;
4-Bromo-3,6-dihydroxy-pyridine-2-carboxylic acid 4-fluoro-benzylamide;
3-Hydroxy-4-methoxy-6-thiophen-2-yl-pyridine-2-carboxylic acid 4-fluoro-benzylamide;
3-Hydroxy-4-methoxy-6-thiazol-2-yl-pyridine-2-carboxylic acid 4-fluoro-benzylamide;
4,6-Dibromo-3-hydroxy-pyridine-2-carboxylic acid 4-fluoro-benzylamide;
6-(4-Fluoro-benzylamino)-hydroxy-4-methoxy-pyridine-2-carboxylic acid 4-fluoro-benzylamide;
5-Hydroxy-4-methoxy-[2,2′]bipyridinyl-6-carboxylic acid 4-fluoro-benzylamide;
3,4,6-Trimethoxy-pyridine-2-carboxylic acid 4-fluoro-benzylamide;
6-Ethyl-3-hydroxy-4-methoxy-pyridine-2-carboxylic acid 4-fluoro-benzylamide;
3-Hydroxy-4-methoxy-6-vinyl-pyridine-2-carboxylic acid 4-fluoro-benzylamide;
3-Hydroxy-4,6-dimethoxy-pyridine-2-carboxylic acid 4-fluoro-benzylamide;
4-Benzyloxy-6-bromo-3-hydroxy-pyridine-2-carboxylic acid 4-fluoro-benzylamide;
6-(1,2-Dihydroxy-ethyl)-3-hydroxy-4-methoxy-pyridine-2-carboxylic acid 4-fluoro-benzylamide;
4-Azido-3-benzyloxy-6-bromo-pyridine-2-carboxylic acid 4-fluoro-benzylamide;
4-Amino-3-benzyloxy-6-bromo-pyridine-2-carboxylic acid 4-fluoro-benzylamide;
4-Amino-6-bromo-3-hydroxy-pyridine-2-carboxylic acid 4-fluoro-benzylamide;
4,6-Dibromo-3-methoxy-pyridine-2-carboxylic acid 4-fluoro-benzylamide;
3-Hydroxy-6-hydroxymethyl-4-methoxy-pyridine-2-carboxylic acid 4-fluoro-benzylamide;
5′-Hydroxy-4′-methoxy-3,4,5,6-tetrahydro-2H-[1,2′]bipyridinyl-6′-carboxylic acid 4-fluoro-benzylamide;
6-(4-Fluoro-benzylcarbamoyl)-5-hydroxy-4-methoxy-pyridine-2-carboxylic acid;
4-Azido-3-benzyloxy-6-bromo-pyridine-2-carboxylic acid 4-fluoro-benzylamide;
6-(2,2-Dimethyl-[1,3]dioxolan-4-yl)-3-hydroxy-4-methoxy-pyridine-2-carboxylic acid 4-fluoro-benzylamide;
3-Hydroxy-4-methoxy-6-(pyridin-2-ylmethoxy)-pyridine-2-carboxylic acid 4-fluoro-benzylamide;
3-Hydroxy-4-methoxy-6-methoxymethyl-pyridine-2-carboxylic acid 4-fluoro-benzylamide;
3-Hydroxy-4-methoxy-6-(tetrahydro-furan-2-yl)-pyridine-2-carboxylic acid benzylamide;
and pharmaceutically acceptable salts thereof.

46. A compound according to claim 1, wherein said compound is selected from:
3-hydroxy-4-methoxy-6-(tetrahydro-furan-2-yl)-pyridine-2-carboxylic acid [2-(4-fluoro-phenyl)-ethyl]-amide;
3-hydroxy-4-methoxy-6-(tetrahydro-furan-2-yl)-pyridine-2-carboxylic acid (pyridin-2-ylmethyl)-amide;
3-hydroxy-4-methoxy-6-(tetrahydro-furan-2-yl)-pyridine-2-carboxylic acid cyclohexylmethyl-amide;
(+)-3-hydroxy-4-methoxy-6-(tetrahydro-furan-2-yl)-pyridine-2-carboxylic acid 4-fluoro-benzylamide;
(−)-3-hydroxy-4-methoxy-6-(tetrahydro-furan-2-yl)-pyridine-2-carboxylic acid 4-fluoro-benzylamide;
4-acetylamino-3-hydroxy-6-(tetrahydro-furan-2-yl)-pyridine-2-carboxylic acid 4-fluoro-benzylamide;
3-hydroxy-4-methanesulfonyl-6-(tetrahydro-furan-2-yl)-pyridine-2-carboxylic acid 4-fluoro-benzylamide;
6-furan-2-yl-3-hydroxy-4-methylsulfanyl-pyridine-2-carboxylic acid 4-fluorobenzylamide;
3-hydroxy-6-methoxy-4-vinyl-pyridine-2-carboxylic acid 4-fluoro-benzylamide;
3-hydroxy-4-phenylacetylamino-6-(tetrahydro-furan-2-yl)-pyridine-2-carboxylic acid 4-fluoro-benzylamide;
6-furan-2-yl-3-hydroxy-4-phenylmethanesulfonylamino-pyridine-2-carboxylic acid 4-fluoro-benzylamide;
3-Hydroxy-4-methoxy-6-(tetrahydro-furan-2-yl)-pyridine-2-carboxylic acid 4-methyl-benzylamide;
3-Hydroxy-4-methoxy-6-(tetrahydro-furan-2-yl)-pyridine-2-carboxylic acid 4-methoxy-benzylamide;
3-Hydroxy-4-methoxy-6-(tetrahydro-furan-2-yl)-pyridine-2-carboxylic acid 4-trifluoromethoxy-benzylamide;
3-Hydroxy-4-methoxy-6-(tetrahydro-furan-2-yl)-pyridine-2-carboxylic acid 4-trifluoromethyl-benzylamide;
3-Hydroxy-4-methoxy-6-(tetrahydro-furan-2-yl)-pyridine-2-carboxylic acid 2-fluoro-benzylamide;
3-Hydroxy-4-methoxy-6-(tetrahydro-furan-2-yl)-pyridine-2-carboxylic acid 3-fluoro-benzylamide;
3-Hydroxy-4-methoxy-6-(tetrahydro-furan-2-yl)-pyridine-2-carboxylic acid 2,4-difluoro-benzylamide;
3-Hydroxy-4-methoxy-6-(tetrahydro-furan-2-yl)-pyridine-2-carboxylic acid 3,4-difluoro-benzylamide;
3-hydroxy-4-methoxy-6-(tetrahydro-furan-2-yl)-pyridine-2-carboxylic acid (4-fluoro-benzyl)-methyl-amide;
3-Hydroxy-4-methoxy-6-(tetrahydro-furan-2-yl)-pyridine-2-carboxylic acid [1-(4-fluoro-phenyl)-ethyl]-amide;
3-Hydroxy-4-methoxy-6-(tetrahydro-furan-2-yl)-pyridine-2-carboxylic acid 4-bromo-benzylamide;
3-Hydroxy-4-methoxy-6-(tetrahydro-furan-2-yl)-pyridine-2-carboxylic acid 4-chloro-benzylamide;
6-(1,1-Dioxo-[1,2]-thiazinan-2-yl)-3-hydroxy-4-methoxy-pyridine-2-carboxylic acid 4-fluoro-benzylamide;
3-Hydroxy-4-methoxy-6-(pyridin-2-yl sulfanyl)-pyridine-2-carboxylic acid 4-fluoro-benzylamide;
3-Hydroxy-4-methoxy-6-methylsulfanyl-pyridine-2-carboxylic acid 4-fluoro-benzylamide;
3-Hydroxy-6-methanesulfonyl-4-methoxy pyridine-2-carboxylic acid 4-fluoro-benzylamide;
3-Hydroxy-4-methoxy-6-(tetrahydrofuran-3-yl)-pyridine-2-carboxylic acid 4-fluoro-benzylamide;
6-Furan-3-yl-3-hydroxy-4-methoxy-pyridine-2-carboxylic acid 4-fluoro-benzylamide;
6-(4-Benzoyl-piperazin-1-yl)-3-hydroxy-4-methoxy-pyridine-2-carboxylic acid 4-fluoro-benzylamide;
3-Hydroxy-4-methoxy-6-morpholin-4-yl-pyridine-2-carboxylic acid 4-fluoro-benzylamide;
3-Hydroxy-4-methoxy-6-(1,3)-oxathioan-2-yl-pyridine-2-carboxylic acid 4-fluoro-benzylamide;
3-Hydroxy-4-methoxy-6-(5-methyl-(1,3)-oxathioan-2-yl)-pyridine-2-carboxylic acid 4-fluoro-benzylamide;
6-(1,3)-Dioxolan-2-yl-3-hydroxy-4-methoxy-pyridine-2-carboxylic acid 4-fluoro-benzylamide;
3-Hydroxy-4-methoxy-6-(4-methyl-(1,3)dioxolan-2-yl)-pyridine-2-carboxylic acid 4-fluoro-benzylamide;
6-(4-Benzyloxymethyl-(1,3)-dioxolan-2-yl)-3-hydroxy-4-methoxy-pyridine-2-calboxylic acid 4-fluoro-benzylamide;
3-Hydroxy-6-(4-hydroxymethyl-(1,3)-dioxolan-2-yl)-4-methoxy-pyridine-2-carboxylic acid 4-fluoro-benzylamide;
6-(1,3)-Dioxan-2-yl-3-hydroxy-4-methoxy-pyridine-2-carboxylic acid 4-fluoro-benzylamide;
3-Hydroxy-4-methoxy-6-(2-methyl-(1,3)-dioxolan-2-yl)-pyridine-2-carboxylic acid 4-fluoro-benzylamide;
6-(4-Fluoro-benzylcarbamoyl)-3-hydroxy-4-methoxy-pyridine-2-carboxylic acid methyl ester;
3-Hydroxy-4-methoxy-pyridine-2,6-dicarboxylic acid bis-(4-fluoro-benzylamide);
3-Hydroxy-4-methoxy-6-(tetrahydro-furan-2-yl)-pyridine-2-carboxylic acid 4-nitro-benzylamide;
3′-Hydroxy-6′-(tetrahydro-furan-2-yl)-3,4,5,6-tetrahydro-2H-(1,4′)bipyridinyl-2′carboxylic acid 4-fluoro-benzylamide;
and pharmaceutically acceptable salts thereof.

47. A compound according to claim 1, wherein said compound is the (+) enantiomer having an enantiomeric excess of 90%.

48. A compound according to claim 1, wherein said compound is the (−) enantiomer having an enantiomeric excess of 90%.

49. A compound according to claim 1, wherein alkyl is methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cyclohexenyl, cyclohex-dienyl, cyclohexyl, trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, or dichlorofluoroethyl.

50. A compound according to claim 1, wherein alkoxy is methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy or neohexyloxy.

51. A compound according to claim 1, wherein aryl is phenyl, tolyl, dimethyphenyl, aminophenyl, anilinyl, naphthyl, anthryl, phenanthryl or biphenyl.

52. A compound according to claim 1, wherein aralkyl is benzyl, benzhydryl, trityl, phenethyl, 3-phenylpropyl, 2-phenylpropyl, 4-phenylbutyl or naphthylmethyl.

53. A compound according to claim 1, wherein aralkyloxy is benzyloxy, benzhydryloxy, trityloxy, phenethyloxy, 3-phenylpropyloxy, 2-phenylpropyloxy, 4-phenylbutyloxy or naphthylmethoxy.

54. A pharmaceutical composition comprising a compound according to claim 1 and at least one pharmaceutically acceptable carrier or excipient thereof.

55. A pharmaceutical composition according to claim 54, further comprising of at least one other antiviral agent.

56. A method of preventing or treating HIV infection in a subject comprising administering to said subject a therapeutically effective amount of a compound according to claim 1.

57. A method according to claim 56, wherein said subject is a human.

58. A method of preventing, delaying or treating AIDS in a subject comprising administering to said subject a therapeutically effective amount of a compound according to claim 1.

59. A method according to claim 58, wherein said subject is a human.

60. A method of inhibiting HIV integrase in a subject comprising administering to said subject a therapeutically effective amount of a compound according to claim 1.

61. A method according to claim 60, wherein said subject is a human.

62. A method of preventing integration of HIV DNA into host cell DNA in a subject comprising administering to said subject a therapeutically effective amount of a compound according to claim 1.

63. A method according to claim 62, wherein said subject is a human.

64. A method of preventing the HIV DNA strand transfer to the host cell DNA in a subject comprising administering to said subject a therapeutically effective amount of a compound according to claim 1.

65. A method according to claim 64, wherein said subject is a human.

66. Use of a compound according to claim 1 for the manufacture of a medicament for preventing or treating HIV infection or preventing, delaying or treating AIDS.
